SEQUENCE LISTING (1 sequence)

SEQ ID NO: 1 - protein, 6 aa, Artificial Sequence
  Description of Artificial Sequence Synthetic peptide
SGGGSG

(19) United States
(12) Patent Application Publication
(10) Pub. No.: US 2021/0380944 A1
(43) Pub. Date: Dec. 9, 2021

(54) THERAPEUTIC PREPARATIONS OF GAMMA-DELTA T CELLS AND NATURAL KILLER CELLS AND METHODS FOR MAKING AND USING THEM

(71) Applicant: Ospedale Pediatrico Bambino GESÙ, Rome (IT)

(72) Inventors: Concetta QUINTARELLI, Rome (IT); Biagio DE ANGELIS, Rome (IT); Franco LOCATELLI, Costa Volpino Bergamo (IT)

(21) Appl. No.: 17/396,487

(22) Filed: Aug. 6, 2021

Related U.S. Application Data
(63) Continuation of application No. 16/557,316, filed on Aug. 30, 2019.
Application No. 16/557,316 is a continuation of application No. PCT/EP2019/070125, filed on Jul. 25, 2019.
(60) Provisional application No. 62/703,654, filed on Jul. 26, 2018.

(51) Int. Cl. C12N 5/0783; C12N 5/00; A61K 35/17; A61K 45/06; A61P 35/02; A61P 35/00

(52) U.S. Cl. CPC C12N 5/0646; C12N 5/0636; C12N 5/0037; A61K 35/17; C12N 2501/2315; A61P 35/02; A61P 35/00; C12N 2501/998; C12N 2501/2302; A61K 45/06

(57) ABSTRACT

Provided are methods of making innate immune cell compositions containing gamma.delta (γδ) T cells and/or Natural Killer (NK) cells, and the resulting compositions and related products of manufacture and kits for use in cancer and infectious disease therapy. The methods provided herein permit tailoring of the relative amounts of gamma.delta (γδ) T cells and Natural Killer (NK) cells in the compositions. for cellular therapies against a wide variety of cancers and infectious diseases. The resulting compositions can further be used to generate compositions containing either NK cells alone or gamma.delta T cells alone, for immune cellular therapies. The compositions provided herein also can be genetically altered: the gamma delta T cells and Natural Killer cells are modified to express chimeric antigen receptors (CARs) or exogenous T cell receptors (TCRs), which can be used to target any cell surface molecule either directly or indirectly, e.g., a marker on a cancer cell or an infected cell.

### RELATED PATENT APPLICATIONS

This patent application is a continuation of international patent application no. PCT/EP2019/070125 filed on Jul. 25, 2019, entitled THERAPEUTIC PREPARATIONS OF GAMMA-DELTA T CELLS AND NATURAL KILLER CELLS AND METHODS FOR MANUFACTURE AND USE, naming Concetta QUINTARELLI et al. as inventors, and designated by attorney docket no. SDH-1001-PC, which claims the benefit of U.S. Provisional Patent Application No. 62/703,654, filed on Jul. 26, 2018, entitled THERAPEUTIC PREPARATIONS OF GAMMA-DELTA T CELLS AND NATURAL KILLER CELLS AND METHODS FOR MAKING AND USING THEM, naming Concetta QUINTARELLI et al. as inventors, and designated by Attorney Docket No. SDH-1001-PV. The entire content of each of the foregoing patent applications is incorporated herein by reference for all purposes, including all text, tables and drawings.

### FIELD

The technology relates in part to innate immune cell compositions for therapy, and to methods of making and using such compositions.

### BACKGROUND

Immune responses to antigen-presenting pathogens and other foreign antigen presenting entities include innate and adaptive defenses. The innate immune response is the first line of immune defense that is active and continuously functioning in the host. Innate immune cells, such as NK cells and gamma.delta (γδ) T cells do not recognize classic HLA antigens. The adaptive immune response is a response that is specifically tailored to an antigen-presenting agent such as a foreign body, cell or microorganism, and often can take several days to mature. Immunotherapies using native or modified (e.g., CAR modified) adaptive immune cells (e.g., alpha.beta (αβ) T cells, dendritic cells, macrophages). For example, αβ T cells expand upon binding to ligands on antigen presenting agents in vivo, which can take days or even weeks. Also, binding of the αβ T cells is through one or more T cell surface molecules or complexes of molecules, including CD4, CD8 and the T cell receptor (TCR), which recognize MHC Class I and Class II ligands encoded by the HLA gene complex (Miceli et al., Semin. Immunol., 3(3):133-141 (1991).

### SUMMARY

Provided herein are compositions relating to immunology and medicine, to methods of making such compositions, and to methods of treatment using such compositions. In certain aspects, provided are compositions, including products of manufacture and kits, and methods, comprising gamma delta T cells (γδ), Natural Killer cells (NK), or combinations of the two, for various cellular therapies. Also provided are methods for making gamma delta T cells (γδ) and Natural Killer cells (NK), which can be genetically altered, for use in these therapies. In alternative aspects, the genetically altered gamma delta T cells (γδ) and Natural Killer cells (NK) are modified to express chimeric antigen receptors (CARs) or exogenous or heterologous T cell receptors (TCRs), which can be used to target any cell surface molecule either directly or indirectly, e.g., a marker on a cancer cell or an infected cell.

Provided in certain aspects are methods for manufacturing a composition containing a population of cells enriched in NK cells and gamma.delta T cells. In certain aspects of the methods provided herein, a sample obtained from a donor (e.g., a tissue, organ or blood sample from healthy subject, or a subject who is a patient to be treated with the population of cells) is exposed to activation conditions that include (a) at least one exogenous polypeptide that immunospecifically binds to a cell adhesion polypeptide, and (b) at least one exogenous polypeptide that immunospecifically binds to a different polypeptide than the cell adhesion polypeptide and is expressed on the surface of one or more cells of the sample; and is exposed to expansion conditions that include contacting the sample with at least one supplemental polypeptide, thereby generating a composition containing a population of cells enriched in NK cells and gamma.delta T cells. A sample sometimes is sequentially exposed to activation conditions and then expansion conditions. The methods provided herein can result in a population of cells having a high level of activation of between about 30% to about 40%, 50%, 60%, 70%, 75%, 80%, 85%, 90% 95% or more of the cells in the population, or at least about 30% 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or more, up to 100% of the cells in the population.

The exogenous polypeptides and supplemental polypeptides used in the methods provided herein can readily be identified and isolated, synthesized or otherwise obtained, including from commercial sources, based in part on available nucleic acid and amino acid sequences and other knowledge of cell adhesion molecules and other immune cell molecules and receptors. In certain aspects, the exogenous polypeptide is human. In some aspects, the exogenous polypeptide is isolated. In certain aspects, the supplemental polypeptide is human. In some aspects, the supplemental polypeptide is isolated.

In certain aspects of the methods provided herein, the sample is depleted of alpha.beta T cells prior to being exposed to activation and expansion conditions, and the resulting depleted cell population sometimes is then be exposed to activation and expansion conditions. In certain aspects, the exogenous polypeptide in (a) above, the exogenous polypeptide in (b) above, or both the exogenous polypeptide in (a) and the exogenous polypeptide in (b) above, are soluble. In some aspects, the exogenous polypeptide in (a) or the exogenous polypeptide in (b) is bound to a solid substrate.

In some aspects of the methods provided herein, at least one supplemental polypeptide is selected such that the amount of NK cells relative to the amount of gamma.delta T cells in the population is dependent on the amount and/or type of the at least one supplemental polypeptide. In certain aspects, the supplemental polypeptide increases or decreases the amount of NK cells relative to gamma.delta T cells in the population of cells after the depleted cell population is contacted with the at least one supplemental polypeptide. In certain aspects, the time for which the sample, or the alpha.beta T cell depleted sample, is exposed to the expansion conditions is selected such that a desired ratio of NK cells to gamma.delta T cells is obtained.

In certain aspects of the methods provided herein, the activation conditions are free of serum from a non-human animal. In some aspects of the methods provided herein, the expansion conditions are free of serum from a non-human animal.

In the methods provided herein, a high percentage of cells often are activated without the use of feeder cells. Sometimes, at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or more, up to 100% of the cells in the population are activated without the use of feeder cells. Thus, in certain aspects of the methods provided herein, the activation conditions are free of feeder cells. In some aspects of the methods provided herein, the expansion conditions are free of feeder cells, and sometimes both the activation and the expansion conditions are free of feeder cells.

Any source of immune cells can be used as a sample, in the methods provided herein. In certain aspects, the sample is selected from among bone marrow, peripheral blood, liver tissue, epithelial tissue and cord blood. In some aspects of the methods provided herein, the sample is not derived from an embryonic source. In certain aspects of the methods provided herein, the sample is peripheral blood and in some aspects, the peripheral blood sample is a processed sample that is processed prior to being subjected to alpha.beta T cell depletion in the methods provided herein. For example, the peripheral blood sample can be processed by density gradient centrifugation to separate and/or isolate a buffy coat containing white blood cells, platelets, granulocytes and the like, which then can be subjected to alpha.beta T cell depletion according to the methods provided herein. In certain aspects, the buffy coat can further undergo a Ficoll gradient separation to obtain mononuclear cells (PBMCs), which then can be subjected to alpha.beta T cell depletion in the methods provided herein. In some aspects, the peripheral blood sample can undergo apheresis to separate the plasma from the cells, and sometimes the cells then are subjected to alpha.beta T cell depletion in the methods provided herein. In certain aspects of the methods provided herein, the sample is cord blood and sometimes the cord blood is processed cord blood that is processed prior to being subjected to alpha.beta T cell depletion in the methods provided herein.

In certain aspects of the methods provided herein, the exogenous polypeptide in (b) immunospecifically binds to a NK cell activation receptor, a gamma.delta T cell activation receptor, or both. Such receptors include, but are not limited to CD2, CD3, CD56, NKp30, NKp44, NKp46, NKG2A, NKG2C, NKG2D, KAR receptors, KIR receptors, SIGLEC-7, KIR3D51, KIR3D51, KIR2DL1 (antibody: 11PB6), DNAM1, NTBA, HLA-DR and the like. In some aspects, the receptor is NKp46. In certain aspects of the methods provided herein, the exogenous polypeptide in (a) immunospecifically binds to CD2. In some aspects, the exogenous polypeptide in (a) or (b), or (a) and (b), is an antibody or an antigen-binding fragment thereof. For purposes herein, recitation of “antibody” includes full-length antibodies and portions thereof including antibody fragments. Antibody fragments include, but are not limited to, Fab fragments, Fab′ fragments, F(ab′)2 fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibody also includes synthetic antibodies, recombinantiy produced antibodies, multispecific antibodies (e.g., bispecific antibodies), human antibodies, non-human antibodies, humanized antibodies, chimeric antibodies, and intrabodies. Antibodies can include members of any immunoglobulin type (e.g., IgG, IgM, IgD, IgE, IgA and IgY), any class (e.g., IgG1, IgG2, IgG3, IgG4, IgA1 and IgA2) or subclass (e.g., IgG2a and IgG2b).

In certain aspects of the methods provided herein, the activation conditions include contacting the sample or depleted cell population with at least two exogenous polypeptides. In some aspects, the first exogenous polypeptide immunospecifically binds to CD2 and the second exogenous polypeptide immunospecifically binds to NKp46. In some aspects, the activation conditions consist or consist essentially of a first exogenous polypeptide that immunospecifically binds to CD2 and a second exogenous polypeptide that immunospecifically binds to NKp46. In certain aspects, the exogenous polypeptide that immunospecifically binds to CD2, the exogenous polypeptide that immunospecifically binds to NKp46, or both the exogenous polypeptide that immunospecifically binds to CD2 and the exogenous polypeptide that immunospecifically binds to NKp46 are soluble. In certain aspects, the first exogenous polypeptide and/or the second exogenous polypeptide is/are an antibody or an antigen-binding fragment thereof.

In certain aspects of the methods provided herein, the expansion conditions include, consist of, or consist essentially of, at least one supplemental polypeptide that is a cytokine and/or a polypeptide that immunospecifically binds to a receptor on a gamma.delta T cell; and/or a portion thereof that immunospecifically binds to a receptor on a gamma.delta T cell. In certain aspects, the cytokine is an interleukin such as, for example, IL-1 (see, e.g., GenBank Accession No. BC008678.1), IL-2 (see, e.g., GenBank Accession No. S77834.1), IL-4 (see, e.g., GenBank Accession No. BC070123.1), IL-7 (see, e.g., GenBank Accession No. BC047698.1), IL-9 (see, e.g., GenBank Accession No. BC066285.1), IL-15 ((see, e.g., GenBank Accession Nos. BC100962.1; 100963.1; 100961.1), IL-21 (see, e.g., GenBank Accession No. LC133256.1), or any combinations thereof. In some aspects of the methods provided herein, the cytokine is IL-2, IL-15 or a combination thereof. In certain aspects, the expansion conditions include, consist of or consist essentially of IL-2, IL-15 and a polypeptide that immunospecifically binds to a receptor on a gamma.delta T cell. In certain aspects, the receptor on the gamma.delta T cell is CD3. In some aspects, the polypeptide that immunospecifically binds to the CD3 receptor on the gamma.delta T cell is an antibody or an antigen-binding fragment thereof and in certain aspects, the antibody is OKT3. In certain aspects, the expansion conditions include contacting the sample or depleted cell population with: (a) an IL-2 polypeptide; (b) an IL-15 polypeptide; (c) an IL-2 polypeptide and an IL-15 polypeptide; (d) an IL-2 polypeptide and an antibody that immunospecifically binds CD3; or (e) an IL-2 polypeptide, an IL-15 polypeptide and an antibody that immunospecifically binds CD3. In some aspects, the antibody that immunospecifically binds CD3 is OKT3.

In certain aspects, the expansion conditions include sequentially exposing the sample (e.g., the source sample or a sample cell population that is alpha.beta T cell depleted) to more than one set of conditions. In some aspects, the sample is exposed to two sets of expansion conditions. In aspects, the cell population exposed to the first set of expansion conditions is washed prior to exposure to the second set of expansion conditions. In certain aspects: (a) the first set of conditions comprise IL-2 and the second set of conditions comprise IL-15; (b) the first set of conditions comprise IL-15 and the second set of conditions comprise IL-2; (c) the first set of conditions comprise IL-2 and an antibody that immunospecifically binds CD3 and the second set of conditions comprise IL-15; or (d) the first set of conditions comprise IL-15 and an antibody that immunospecifically binds CD3 and the second set of conditions comprise IL-2 and an antibody that immunospecifically binds CD3.

In certain aspects of the methods provided herein, the first set of expansion conditions including one or more supplemental polypeptides often results in a first cell population comprising a first ratio of NK cells to gamma.delta T cells, which then can be fine-tuned to a desired final ratio of NK cells to gamma.delta T cells using a second set of expansion conditions, where the first set of conditions is different than the second set of conditions.

In the methods provided herein, a cell population can be exposed to activation and expansion conditions simultaneously or sequentially in any order. Further, the exogenous polypeptide(s) can function as supplemental polypeptide(s) and/or vice versa.

The immune cell compositions obtained by the methods provided herein are enriched in NK cells and gamma.delta T cells, which are innate immune cells, relative to the composition of immune cells in nature (e.g., biological fluids and tissues). In nature, alpha.beta T cells, which are adaptive immune cells, are found in much higher amounts than NK cells and gamma.delta T cells. On the other hand, in the compositions provided herein, alpha.beta T cells are absent or present in negligible to low amounts, with NK cells and gamma.delta T cells being the predominant immune cell components.

Depending on the expansion conditions, the compositions enriched in innate immune cells that are obtained by the methods provided herein can contain different amounts of NK cells relative to gamma.delta T cells. In certain non-limiting examples: (i) when the supplemental polypeptide is IL-2, the resulting population of cells enriched in NK cells and gamma.delta T cells often contains about 25-30% NK cells and about 70-75% gamma.delta T cells; (ii) when the supplemental polypeptide is IL-15, the resulting population of cells enriched in NK cells and gamma.delta T cells often contains about 80-99% NK cells and about 1-20% gamma.delta T cells; (iii) when the supplemental polypeptide is IL-2 and an antibody that immunospecifically binds CD3, such as OKT3, the resulting population of cells enriched in NK cells and gamma.delta T cells often contains about 40-45% NK cells and about 55-60% gamma.delta T cells; (iv) when the supplemental polypeptide is IL-2 until Day 20 of the expansion conditions and then switched to IL-15 until Day 30, compared to treatment with IL-2 alone, the percentage of gamma.delta T cells often increases from about 50% to about 70%, often with a corresponding decrease in the percentage of NK cells; and (v) when the supplemental polypeptide is IL-15 until Day 20 of the expansion conditions and then switched to IL-2 until Day 30, compared to treatment with IL-15 alone, the percentage of NK cells often increases from about 80% to about 90%, often with a corresponding decrease in the percentage of gamma.delta T cells.

In certain aspects of the methods provided herein, the sample or the depleted cell population are not exposed to conditions that select for NK cells or gamma.delta T cells. In some aspects, the sample or the depleted cell population are not exposed to conditions that deplete cells other than the alpha-beta T cells.

The cells of the compositions prepared by the methods provided herein can further be genetically modified to express an exogeneous polynucleotide, such as, for example, a tumor necrosis factor receptor, a chimeric antigen receptor (CAR), a myeloid differentiation primary response protein or an innate immune signal transduction adaptor. The cells also can be modified to mutate one or more polypeptides or to delete one or more polypeptides.

In certain aspects of the methods provided herein, the compositions containing gamma.delta cells and NK cells can be subjected to a treatment whereby compositions consisting of, or consisting essentially of, either NK cells or gamma.delta cells are obtained. The treatment can be a depletion, e.g., obtaining NK cells by depleting all gamma.delta (CD3+) cells using an anti-CD3 antibody, or a positive selection, e.g., selecting for the gamma.delta T cells using an anti-CD3 antibody. In some aspects, the anti-CD3 antibody, such as an OKT3 antibody, can be bound to a solid phase.

In aspects of the methods provided herein, the activation conditions, expansion conditions, or activation and expansion conditions include incubation of the sample or depleted cell population in a feeder cell free medium for about 1 hour, 2 hours, 5 hours, 10 hours, 12 hours, 15 hours, 20 hours, or days such as 1, 2, 3, 4, 5, 6, 7, 8, 9, 10, 11, 12, 13, 14, 15, 16, 17, 18, 19, 20, 21, 25, 30, 35, 40, 45, 50, 55 or 60 or more days or weeks such as about 2, 3, 4, 5, 6, 7, 8, 9 or 10 weeks. In certain aspects, the activation conditions are for a period of about 1 hour, 5 hours, 10 hours, 12 hours, 15 hours or 20 hours to about 1 day, 2 days, 3 days, 4 days, 5 days, 6 days or 1 week. In some aspects, the activation conditions are for a period of between about 12 hours, 24 hours, 36 hours, or 2 days to about 3 days, 4 days, 5 days, 6 days or 1 week, or about 2 days to about 4 days or 5 days, or about 3 days to about 4 days. In certain aspects, the expansion conditions are for a period of 1 day, 2 days, 3 days, 4 days, 5 days, 6 days or 1 week. In certain aspects, the expansion conditions are performed in sequential cycles, and each cycle independently is performed for about 1 day, 2 days, 3 days, 4 days, 5 days, 6 days, 7 days or 1 week, 2 weeks, 3 weeks or more. In some aspects, the number of expansion cycles are greater than one, for example, 2, 3, 4, 5, 6, 7, 8, 9 or 10 more cycles. In certain aspects, each expansion cycle is for about 7 days. In some aspects, the number of expansion cycles is 3.

In some aspects, the expanded population of cells enriched in NK cells and gamma.delta T cells is free of exhausted cells. In certain aspects, the expanded population of cells enriched in NK cells and gamma.delta T cells is free of exhausted cells after 60 days of the expansion conditions.

In certain aspects, the population of cells enriched in NK cells and gamma.delta T cells, which are obtained by the methods provided herein, contains 80% or more innate immune cells. In some aspects, between about 80% to about 100%, or at least about 80%, 81%, 82%, 83%, 84%, 85%, 86%, 87%, 88%, 89%, 90%, 91%, 92%, 93%, 94%, 95%, 96%, 97%, 98%, 99% or more, up to 100% of the cells are innate immune cells.

In some aspects of the methods provided herein, the activation conditions, the expansion conditions, or both the activation and expansion conditions, do not comprise a bisphosphonate. Exemplary bisphosphonates include, but are not limited to, clodronate, etidronate, alendronate, pamidronate, zoledronate (zoledronic acid), neridronate and the like.

In certain aspects of the methods provided herein, the compositions obtained by the methods provided herein contain gamma.delta T cells that are polyclonal with respect to V.delta.1 and V.delta.2 expression. The relative amounts of the V.delta.1 and V.delta.2 cells can be tailored by the expansion conditions such as one or more of the absence of bisphosphonates, the choice of supplemental polypeptide, and the time period for which the sample or depleted cell population is subjected to expansion conditions. In certain non-limiting examples, when the expansion conditions include IL-2 and a polypeptide that immunospecifically binds to CD3 (e.g., OKT3), (i) the V.delta.1 cells can be between about 75% to about 95% of the gamma.delta T cells and sometimes between about 80% to about 90% of the gamma.delta T cells, and (ii) the V.delta.2 cells can be between about 10% to about 25% of the gamma.delta T cells and sometimes between about 10% to about 15% or 20% of the gamma.delta T cells. In certain aspects, when the expansion conditions include IL-2 (e.g., without IL-15 and with no polypeptide that immunospecifically binds to CD3), (i) the V.delta.1 cells can be between about 30% to about 60% of the gamma.delta T cells and sometimes between about 35% to about 55% of the gamma.delta T cells, and (ii) the V.delta.2 cells can be between about 35% to about 60% of the gamma.delta T cells and sometimes between about 40% to about 50% or 55% of the gamma.delta T cells. In certain aspects, when the expansion conditions include IL-15 (e.g., without IL-2 and with no polypeptide that immunospecifically binds to CD3), (i) the V.delta.1 cells can be between about 10% to about 30% of the gamma.delta T cells and sometimes between about 20% to about 25% of the gamma.delta T cells, and (ii) the V.delta.2 cells can be between about 65% to about 80% of the gamma.delta T cells and sometimes between about 70% to about 75% or 80% of the gamma.delta T cells.

Provided herein in certain aspects are compositions containing a population of cells, where the population includes: a plurality of NK cells and a plurality of gamma.delta T cells; and is alpha.beta T cell depleted. In some aspects, the composition is free of feeder cells. Also provided herein in certain aspects are compositions containing a population of cells, where the population includes: a plurality of NK cells and a plurality of gamma.delta T cells; is alpha.beta T cell depleted; and is free of feeder cells. In certain aspects, the population of cells is a modified population of peripheral blood cells. In some aspects, the compositions provided herein contain: (i) between about 25% to about 45% NK cells and between about 55% to about 75% gamma.delta T cells; (ii) between about 25% to about 30% NK cells and between about 70% to about 75% gamma.delta T cells; (iii) between about 80% to about 99% NK cells and between about 1% to about 20% gamma.delta T cells; or (iv) between about 40% to about 45% NK cells and between about 55% to about 60% gamma.delta T cells.

In certain aspects, 30% or more of the cells in the compositions provided herein are activated. In some aspects, between about 30% to about 40%, 50%, 60%, 70%, 75%, 80%, 85%, 90% 95% or more of the cells in the population, or at least about 30% 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or more, up to 100% of the cells in the population.

In some aspects, the compositions provided herein contain a population of cells that include one or more of the following activation markers as a percentage of the total number of cells in the population: (a) 90% or more of the cells in the population express KIR5; (b) 10% or more of the cells in the population express SIGLEC-7; (c) 60% or more of the cells in the population express KIR3D51; (d) 10% or more of the cells in the population express KIR2DL1; (e) 25% or more of the cells in the population express NKp30, NKp44 and/or NKp46; (f) 35% or more of the cells in the population express NKG2D; (g) 90% or more of the cells in the population express DNAM1; (h) 85% or more of the cells in the population express NTBA; and (i) 95% or more of the cells in the population express CD2.

In certain aspects, the compositions provided herein contain 80% or more innate immune cells. In some aspects, the compositions are enriched in activated cytotoxic cells that are CD56+; and in certain aspects, between about 80% to about 100%, or at least about 80%, 81%, 82%, 83%, 84%, 85%, 86%, 87%, 88%, 89%, 90%, 91%, 92%, 93%, 94%, 95%, 96%, 97%, 98%, 99% or 100% of the cells are CD56+. In some aspects, the compositions provided herein are enriched in activated cytotoxic cells that are CD57-. In certain aspects, the compositions provided herein are enriched in activated cytotoxic cells that are CD56+CD57-. In some aspects, between about 10% to about 40%, or at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the cells are CD16+. In certain aspects, less than 5%, less than 4%, less than 3% or less than 2% of the cells in the compositions provided herein are CD57+.

In certain aspects, the compositions provided herein are substantially free of cells other than NK cells and gamma.delta T cells. In some aspects, the compositions provided herein contain about or less than 5%, 4%, 3%, 2%, 1%, 0.9%, 0.8%, 0.7%, 0.6%, 0.5%, 0.4%, 0.3%, 0.2%, 0.1% or less NKT cells and/or about or less than 5%, 4%, 3%, 2%, 1%, 0.9%, 0.8%, 0.7%, 0.6%, 0.5%, 0.4%, 0.3%, 0.2%, 0.1% or less alpha.beta T cells. In certain aspects, a subset of NK cells in the compositions are CD16+ cells. In some aspects, the majority of gamma.delta T cells or the majority of the NK cells, or the majority of both the gamma.delta T cells and the NK cells, are CD57− cells.

In certain aspects, the gamma.delta T cells of the compositions provided herein are polyclonal with respect to V.delta.1 and V.delta.2 expression. In some aspects, the gamma.delta T cell populations of the compositions provided herein contain: (i) between about 75% to about 95% V.delta.1 cells, for example between about 80% to about 90% V.delta.1 cells; and between about 5% to about 25% V.delta.2 cells, for example between about 10% to about 15% or 20% V.delta.2 cells; or (ii) between about 30% to about 60% V.delta.1 cells, for example between about 35% to about 55% V.delta.1 cells and between about 35% to about 60% V.delta.2 cells, for example between about 40% to about 50% or 55% V.delta.2 cells; or (iii) between about 10% to about 30% V.delta.1 cells, for example between about 20% to about 25% V.delta.1 cells and between about 65% to about 80% V.delta.2 cells, for example between about 70% to about 75% or 80% V.delta.2 cells.

In certain aspects, about 50% to about 99% or more, or greater than or equal to about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99% 80%, 81%, 82%, 83%, 84%, 85%, 86%, 87%, 88%, 89%, 90%, 91%, 92%, 93%, 94%, 95%, 96%, 97%, 98%, 99% or fractions thereof, up to 100%, of the NK cells and/or the gamma.delta T cells of the compositions provided herein are CD8+. In some aspects, less than 2% of the NK cells and/or the gamma.delta T cells are CD4+. In aspects, less than 2% of the NK cells and/or the gamma.delta T cells are CD8+CD4+. In certain aspects, a fraction of between about 15% to about 30% of the NK cells and/or between about 55% to 85% the gamma.delta T cells are CD8−CD4−.

In certain aspects of the compositions provided herein, between about 30% to about 99% or more, or at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or fractions thereof, up to 100% of the cells in the population further comprise a genetic modification comprising an exogenous polynucleotide, a mutated polynucleotide, a deleted polynucleotide or combinations thereof. In some aspects, the genetic modification includes an exogenous polynucleotide. The exogenous polynucleotide sometimes is in a retroviral vector or a lentiviral vector and, sometimes, the exogenous polynucleotide is integrated into genomes of one or more cells of the modified cell population.

The exogenous polynucleotide can, in certain aspects, encode an exogenous or heterologous T-cell receptor, a tumor necrosis factor receptor, a chimeric antigen receptor (CAR), a myeloid differentiation primary response protein, an innate immune signal transduction adaptor or other protein or polypeptide of interest and can, in some aspects, include a promoter or other regulator of gene expression. In some aspects, the exogenous polynucleotide is a regulatory sequence, such as a promoter or enhancer.

In certain aspects, the exogenous polynucleotide encodes a chimeric antigen receptor (CAR) and the cells in the composition comprise a CAR. CARs are recombinant receptors that provide both antigen-binding and T cell activating functions (see, e.g., Sadelain et al., Cancer Discov., 3(4):388-398 (2013)). When immune cells, such as T cells, are engineered (genetically modified) to express a CAR, it provides the immune cells a new and/or improved ability to target a protein or antigen of interest. The target protein or antigen of interest can, in certain aspects, be a cancer antigen or an infectious disease antigen, several of which are known and/or identifiable in the art. In some aspects, the CAR contains a binding molecule portion that immunospecifically binds to one or more of CD19 (see, e.g., GenBank Accession No. AH005421.2), GD2 (a disialoganglioside; see, e.g., Schulz et al., Cancer Res., 44(12):5914-5920 (1984)), HER3 (see, e.g., GenBank Accession No. M34309.1), B7H3 (see, e.g., GenBank Accession No. BC062581.1), CD123 (see, e.g., GenBank Accession Nos. BC035407.1; BX296563.3) or CD30 (see, e.g., GenBank Accession Nos. M83554.1; AY498860.1).

Any of the compositions provided herein can further be treated to remove either the gamma.delta T cells or the NK cells, thereby generating a composition containing substantially all NK cells or substantially all gamma.delta T cells. For example, the compositions provided herein can be treated with an anti-CD3 antibody to either deplete the gamma.delta T cells from the mixture of NK cells and gamma.delta T cells, leaving behind a composition containing substantially all or all NK cells, or, alternately, the anti-CD3 antibody can be used to isolate a population of substantially pure gamma.delta T cells from the mixture.

Provided in certain aspects are therapeutic compositions (or therapeutic combinations) of cells comprising: a plurality of gamma delta T cells (γδ); a plurality of Natural Killer cells (NK); or a combination of γδ and NK cells. In certain aspects, the γδ and/or NK cells are recombinantly engineered or genetically modified, where, optionally, the γδ and/or NK cells are recombinantly engineered or genetically modified to express extracellularly an exogenous or heterologous protein, and, optionally, the exogenous, heterologous or chimeric protein is a chimeric antigen receptor (CAR) or an exogenous or heterologous T cell receptor (TCR), and optionally the exogenous, heterologous or chimeric protein or CAR is specific for (can specifically bind to) a cancer cell or tumor marker or an infected cell, or the exogenous, heterologous or chimeric protein or CAR is specific for (can specifically bind to) an antibody that can specifically target and bind to a cancer cell or tumor marker, or an infected cell, or any disease associated antigen.

In some aspects, the γδ and/or NK cells are human cells or animal cells. In certain aspects, the therapeutic compositions (or therapeutic combinations) are formulated for intravenous (IV), intrathecal, intramuscular (IM), intraperitoneal (IP) or intratumoral (IT) administration, into the joint space or is injected or implanted at or near the site of the cancer or infection, or is formulated in a unit dosage form, where, optionally, the unit dosage comprises between about 10² to 10¹² cells. or intramuscular (IM) administration, or is formulated in a unit dosage form, where, optionally, the unit dosage comprises between about 10² to 10¹² cells.

In some aspects, the γδ and/or NK cells are isolated from an in vivo source. In some aspects, the γδ and/or NK cells are expanded in culture, or are isolated from an in vivo source and expanded in culture. In certain aspects, the γδ and/or NK cells are isolated from an in vivo source and expanded without using any feeder cells, or without using a feeder cell layer, in culture, thereby generating an expanded population of γδ and/or NK cells lacking feeder cells. In some aspects, the γδ and/or NK cells are isolated from an in vivo source and expanded using feeder cells or a feeder cell layer in culture, where, optionally, the feeder cells are substantially removed and/or killed to generate an expanded population of γδ and/or NK cells substantially lacking feeder cells.

In certain aspects, the in vivo source of γδ and/or NK cells is from an autologous source (optionally, from an individual to be the recipient of the γδ and/or NK cells), or an exogenous, heterologous or an allogeneic source.

Also provided herein in certain aspects are pharmaceutical compositions containing any of the compositions provided herein and a pharmaceutically acceptable carrier.

In certain aspects, provided herein are methods of making genetically modified immune cells by adding an exogenous polynucleotide to the compositions provided herein, mutating a polynucleotide in one or more cells of the compositions provided herein, or deleting a polynucleotide in one or more cells of the compositions provided herein. In some aspects, the genetic modification is an exogenous polynucleotide. The methods used to manufacture the compositions provided herein often result in cells with a high state of activation that facilitates, for example, the introduction of an exogenous polynucleotide by retroviral or lentiviral transduction. In certain aspects, the methods of making genetically modified immune cells provided herein often result in compositions where between about 30% to about 99% or more, or at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or fractions thereof, up to 100% of the cells comprise the genetic modification.

Provided herein in certain aspects are kits containing any of the compositions or pharmaceutical compositions provided herein, optionally, instructions for use and, optionally, a cytokine. The compositions, pharmaceutical compositions or kits provided herein can be stored at refrigeration temperatures e.g., 10 degrees Celsius or less, for example, 9, 8, 7, 6, 5, 4, 3, 2, 1 up to negative 4 degrees Celsius or less) or freezing temperatures (e.g., negative 15, 20, 25, 30, 35, 40, 45, 50, 55, 60, 65, 70, 75, 80, 85 degrees or less) as necessary for storage and/or transportation. In certain aspects, the kits contain between about 1×10⁵ cells to about 1×10¹² cells, for example about 1×10⁶, 1×10⁷, 1×10⁸, 1×10⁹ or 1×10¹⁰ cells. In certain aspects, the kits provided herein can include a cytokine. In some aspects, the cytokine is selected from among one or more of TNF (see, e.g., GenBank Accession Nos. KJ892290.1; AY214167.1), IFNγ (see, e.g., GenBank Accession No. J00219.1), interleukins IL-1s, IL-2, IL-4, IL-6, IL-7, IL-10 (see, e.g., GenBank Accession No. U16720.1), IL-12 (see, e.g., GenBank Accession No. AF404773.1 for IL-12A; see, e.g., GenBank Accession No. AF512686.1 for IL-12B), IL-15, IL-18 (see, e.g., GenBank Accession Nos. BC007461.1; BC007007.1), IL-21, CCL4 (see, e.g., GenBank Accession Nos. CR542119.1; KJ901727.1; KJ901726.1), RANTES (see, e.g., GenBank Accession No. GQ504011.1), and TGFβ (see, e.g., National Center for Biotechnology Information (NCBI) Accession No. NM_000660.7). In certain aspects, the kits contain the compositions or pharmaceutical compositions provided herein in a unit dosage form.

Provided in certain aspects are products of manufacture and kits for practicing methods as provided herein, including a therapeutic combination of cells as provided herein. In certain aspects, the products of manufacture and kits further comprise instructions for practicing the methods as provided herein. In some aspects, the products of manufacture and kits further comprise an antibody capable of specifically binding a cancer-associated or tumor-associated, an infection-associated or a disease-associated antigen. Products of manufacture as provided herein can comprise implants comprising therapeutic combination of cells as provided herein.

Also provided herein in certain aspects are methods for treating a cancer or an infection by administering to a subject in need thereof any one of the compositions, pharmaceutical compositions or kits provided herein in an amount effective to treat the cancer or infection. The treatment can be administered in an autologous setting or in an allogeneic setting. In some aspects, the donor of the sample from which the composition, pharmaceutical composition or kit is produced is the recipient of the treatment. In certain aspects, the treatment can be administered on two or more separate days and in certain aspects, the treatment can be administered in multiple doses. In certain aspects, the treatment is administered at between about 1 unit dosage to about 36 or more unit dosages at intervals of between about 2 weeks to about 4 weeks. In some aspects, the treatment is administered as a single unit dosage one, two, three, four or up to five times daily, or one, two, three, four, five, six, seven, eight, nine or ten or more times over the course of several days, weeks or months, or every other day, or one, two, three four, five or six times a week. The treatment can be administered intravenously (IV), intrathecally or intramuscularly (IM), intraperitoneally (IP), intra-pleurally, into the joint space or is injected or implanted at or near the site of the cancer or infection, and at a unit dosage of between about 10⁴ to about 10¹⁰ cells per kilogram of weight of the subject, or between about 10⁶ to about 10¹² cells per subject. In certain aspects, the unit dosage is about 10¹⁰ cells per subject, or about 10⁸ cells per kilogram of weight of the subject.

In certain aspects of the methods of treatment provided herein, the treatment is for cancer. In some aspects, the cancer is selected from among a lung cancer, melanoma, breast cancer, prostate cancer, colon cancer, renal cell carcinoma, ovarian cancer, neuroblastoma, rhabdomyosarcoma, leukemia or lymphoma, Hodgkin's lymphoma or childhood acute lymphoblastic leukemia, non-Hodgkin lymphoma, a mastocytoma or a mast cell tumor, an ovarian cancer or carcinoma, pancreatic cancer, a non-small cell lung cancer, small cell lung cancer, hepatocarcinoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a colon carcinoma, cervical carcinoma, sarcoma, bladder tumor, tumor of the reticuloendothelial tissues, Wilm's tumor, a bone cancer, an osteosarcoma, a renal cancer, or head and neck cancer, oral cancer, a laryngeal cancer, metastatic disease or an oropharyngeal cancer.

In certain aspects of the methods of treatment provided herein, a second agent is co-administered with the composition, pharmaceutical composition or kit. In some aspects, the second agent is an antibody that immunospecifically binds to a cancer-associated antigen. In certain aspects, the cancer-associated antigen is selected from the group consisting of α-fetoprotein (AFP), σ-actinin-4, A3, antigen specific for A33 antibody, ART-4, B7, B7-H3, Ba 733, BAGE, BrE3-antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CDC27, CDK-4/m, CDKN2A, CTLA4, CXCR4, CXCR7, CXCL12, HIF-1a, colon-specific antigen-p (CSAp), CEA (CEACAM-5), CEACAM-6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and subunits thereof, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), GD2, MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD1 receptor, placental growth factor, p53, PLAGL2, prostatic acid phosphatase, PSA, PRAME, PSMA, PIGF, ILGF, ILGF-R, L-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, and Kras.

In certain aspects, the cells of the compositions provided herein can be engineered to express an antibody that binds to a cancer-associated antigen. In some aspects, the antibody is co-administered as a second agent. In certain aspects, the cancer-associated antigen is selected from among hR1 (anti-IGF-1R), hPAM4 (anti-mucin), KC4 (anti-mucin), hA20 (anti-CD20), hA19 (anti-CD19), hIMMU31 (anti-AFP), hLL1 (anti-CD74), hLL2 (anti-CD22), anti-CD19/CD22 bispecific antibody, RFB4 (anti-CD22), hMu-9 (anti-CSAp), hL243 (anti-HLA-DR), hMN-14 (anti-CEACAM-5), hMN-15 (anti-CEACAM-6), hRS7 (anti-TROP-2), hMN-3 (anti-CEACAM-6), CC49 (anti-TAG-72), J591 (anti-PSMA), D2/B (anti-PSMA), G250 (anti-carbonic anhydrase IX), dinutuximab (anti-GD2), infliximab (anti-TNF-α), certolizumab pegol (anti-TNF-α), adalimumab (anti-TNF-α), alemtuzumab (anti-CD52), bevacizumab (anti-VEGF), cetuximab (anti-EGFR), gemtuzumab (anti-CD33), ibritumomab tiuxetan (anti-CD20), panitumumab (anti-EGFR), rituximab (anti-CD20), tositumomab (anti-CD20), GA101 (anti-CD20), trastuzumab (anti-HER2/neu), tocilizumab (anti-IL-6 receptor), basiliximab (anti-CD25), daclizumab (anti-CD25), efalizumab (anti-CD11a), muromonab-CD3 (anti-CD3 receptor), natalizumab (anti-α4 integrin), BWA-3 (anti-histone H2A/H4), LG2-1 (anti-histone H3), MRA12 (anti-histone H1), PR1-1 (anti-histone H2B), LG11-2 (anti-histone H2B), and LG2-2 (anti-histone H2B).

In certain aspects of the methods of treatment provided herein, the treatment is for an infection. In some aspects, the infection is characterized by the presence of a bacterial, fungal, viral or protozoan pathogen. In aspects of the methods of treatment provided herein, the infection is selected from the group consisting of Herpes, ebola, West Nile virus, Vaccinia virus, Epstein Barr virus, Hepatitis A Virus (HAV); Hepatitis B Virus (HBV); Hepatitis C Virus (HCV); herpes viruses (e.g. HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococca, Rickettsia bacteria, Salmonella, Serratia, Staphylococci, Streptococci, tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia lambia, trichinella, Dientamoeba fragilis, Trypanosoma brucei, Trypanosoma cruzi, or Leishmania donovani.

In some aspects, provided herein are methods for treating a cancer, a tumor, a dysfunctional cell or an infected cell, comprising: (a) administering to an individual in need thereof a therapeutically effective amount of a therapeutic composition of cells described herein, or (b) (i) providing or having provided a therapeutic composition of cells describes herein; and, (ii) administering or having administered to an individual in need thereof a therapeutically effective amount of the therapeutic composition of cells.

In certain aspects of the methods of treatment provided herein, the individual in need thereof is a human or an animal. In some aspects, the γδ and/or NK cells are isolated from an in vivo source, and optionally the in vivo source γδ and/or NK cells is from a syngeneic or an autologous source (optionally, from an individual to be the recipient of the γδ and/or NK cells), or an exogenous, heterologous or an allogeneic source, or a combination thereof. In aspects of the methods provided herein, the therapeutic compositions or combinations of cells is administered intravenously (IV), intrathecally or intramuscularly (IM), or is injected or implanted in or near (approximate to) the cancer, tumor, dysfunctional or infected cell, and optionally the therapeutic composition of cells is delivered in implant or a gel, where, optionally, the gel is a hydrogel.

In some aspects of the methods of treatment provided herein, the therapeutic compositions or combinations of cells is administered in a unit dosage form, where, optionally, the unit dosage comprises between about 10² to 10¹² cells, or 104 to 10¹⁰ cells; or the daily dosage comprises between about 10² to 10¹² cells, or 104 to 10¹⁰ cells. In certain aspects, the therapeutic compositions or combinations of cells, or unit dosage forms, are administered several (a plurality of) times, or two, three, four, five, six, seven, eight, nine or ten or more times, to the individual in need thereof over a course of several days, weeks or months, and optionally each of the plurality of unit dosage forms is administered: daily; every other day; 2, 3, 4, 5, or 6 times a week; or, once a week.

In certain aspects of the methods of treatment provided herein, the individual in need of treatment is first induced to initiate an immune response to a cancer, infection or disease by pre-dosing the individual in need thereof with an unconjugated antibody against a cancer-associated or tumor-associated, an infection-associated or a disease-associated antigen, followed by administration of a therapeutic composition of cells described herein, where at least some of the therapeutic composition of cells expresses on its cell surface a polypeptide, optionally a CAR, that specifically binds to the unconjugated antibody. In some aspects, the individual in need thereof is administered an antibody capable of specifically binding a cancer-associated or tumor-associated, an infection-associated or a disease-associated antigen, followed by administration of a therapeutic composition of cells described herein, where at least some of the therapeutic composition of cells expresses on its cell surface a polypeptide, optionally a CAR, that specifically binds to the antibody, and optionally the antibody is administered before, with, or after administration of the therapeutic composition of cells.

The details of certain embodiments of the technology are set forth in the accompanying drawings and the description below. Other features, objects, and advantages of the technology will be apparent from the description and drawings, and from the claims.

All publications, patents, patent applications cited herein are hereby expressly incorporated by reference for all purposes.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows the expansion of the αβTCRneg mononuclear cells as a function of days the cells were activated with antibodies and expanded in the feeder-free culture conditions as analyzed on day 10 of culture.

FIG. 2 shows an analysis of the composition of an activated expanded αβTCRneg mononuclear cell population (referred to herein as an INNATE-K or BINATE cell population).

FIG. 3 shows the frequency and distribution of T cell lineage markers, CD4 (helper) and CD8 (cytotoxic), on the γδ TCR+ cells in a day 10 sample of the feeder-free, culture expanded activated αβTCRneg cells as analyzed by flow cytometry.

FIG. 4 shows an analysis of expansion rate of CAR.19 INNATE-K cells over 10 days.

FIG. 5 shows data from a co-culture cytotoxicity assay, where INNATE-K cells and INNATE-CAR.19 cells were co-cultured with CD19+ leukemia (221) or CD19+ lymphoma (Daudi) cell lines.

FIG. 6 shows total numbers of an expanded INNATE-NK cell population in plates over prolonged in vitro culture.

FIG. 7 shows a comparison of total cell numbers of plate versus bioreactor expanded INNATE-NK populations.

FIG. 8 shows a subset cell composition during different time points of an in vitro INNATE-NK expansion.

FIG. 9 shows chimeric antigen receptor (CAR) molecule expression in INNATE-NK CAR cells during prolonged in vitro culture.

FIG. 10 shows expression of activation and cytolytic molecules after expansion of INNATE-NK and INNATE-NK CAR population.

FIG. 11 shows lack of exhaustion in feeder-free expanded INNATE-NK cells and INNATE-NK CAR cells.

FIG. 12 shows a cytotoxic co-culture assay with INNATE-NK or INNATE-NK-CAR.19 and 4 tumor cell lines. Row A: 221, a CD19+ leukemia cell line; Row B: Daudi, a CD19+ lymphoma cell line; Row C: BV173, a CD19+(variable expressing) pre-B tumor cell line; and Row D: KARPAS, a CD19-tumor cell line.

FIG. 13 and FIG. 14 show % specific lysis of primary tumor cells as a function of the ratio of effector (E) to target (T), for both INNATE-NK cells and INNATE-NK-CAR.19 cell populations in different test runs.

FIG. 15 shows percentage residual primary CD19+ tumor after the co-culture with the effector cells (INNATE-NK cells and INNATE-NK-CAR.19 cell populations) compared to the control condition in which primary CD19+ leukemia blasts were plated in the absence of effector cells FIG. 16 shows a survival curve of animals receiving INNATE-NK and INNATE-NK-CAR.19 cells.

FIG. 17 shows total BINATE expansion expressed by total cell number over time (i.e., activated as TCR neg cell expansion in flasks in either IL-2 or IL-15 supplemented BINATE medium, and IL-15 expansion of activated αβ TCRneg − cell population in a bioreactor).

FIG. 18 shows total BINATE expansion expressed by total cell number over time (i.e., activated as TCRneg cell expansion in flasks in either IL-2, IL-15, IL-2/OKT3, IL-2/IL-15, or IL-2/IL-15/OKT3 supplemented BINATE medium).

FIG. 19 shows a phenotypic analysis by marker type (activation, cytotoxicity and exhaustion/immaturity) for IL-15 expansion conditions.

FIG. 20 shows a phenotypic analysis by marker type (activation, cytotoxicity and exhaustion/immaturity) for IL-2 expansion conditions.

FIG. 21 shows a phenotypic analysis by marker type (activation, cytotoxicity and exhaustion/immaturity) for IL-2/OKT3 expansion conditions.

FIG. 22 shows cell numbers recorded for BINATE and BINATE.CARGD2 populations approximately every 7 days.

FIG. 23 shows in vivo maintenance of human BINATE cells in both mouse blood and mouse liver.

FIG. 24 shows a flowchart illustrating example variations of a process for isolating a pure NK cell population and/or isolating a pure γδT (gd) cell population.

### DETAILED DESCRIPTION

In certain aspects, provided herein are therapeutic immune cell compositions in which a significant number of immune cells, or majority of immune cells, are innate immune cells. Such compositions can generate an innate immune response in a subject after administration. The innate immune system is active and continuously functioning in the host, responds quickly (within minutes to hours after infection), and responds in a non-specific fashion that provides the potential to be used as an “off-the-shelf” immunotherapy against a wider patient population. Innate immune cells, such as NK cells and gamma.delta (γδ) T cells, do not recognize classic HLA antigens and therefore can be used in an off-the-shelf (allogeneic) setting, while mitigating graft versus host disease (GvHD). Innate immune cells can be expanded ex vivo, thereby avoiding cytokine release syndrome (CRS). Innate immune cell compositions, in which components (e.g., NK cells, gamma.delta (γδ) T cells) and amounts of components can be tailored, can be utilized to provide a synergistic line of attack against a variety of cancers, including solid tumors and hematological cancers, infections and the like.

In contrast, certain immune cell compositions not generated by methods described herein include a majority of adaptive immune cells instead of innate immune cells. Recognition is through HLA antigens when utilizing an immune cell composition in which a majority of cells are adaptive immune cells, and there is a significant risk of GvHD, particularly if the adaptive immune cells used in the immunotherapy are from an allogeneic source. The adaptive immune cells utilized in such compositions therefore are most often derived from the patient/subject (autologous) or from a designated “matched” donor, which limits the repertoire of the immune therapy, i.e., the ability to use it in an “off-the-shelf” manner on a patient population at large. Most cell therapies utilizing such compositions are autologous, or at the least specific donor derived. This means that each patient's dose must be individually tailored for that patient. Thus, current T cell-based therapeutic compositions, for example, cell therapies using chimeric antigen receptors (CARs) or exogenous T cell receptors (TCRs) (as in αβ T cells), cannot be used “off the shelf”. The manufacturing process can take six weeks or more, during which time the patients' disease, e.g., cancer or infection, may have progressed. Also, some of these patient-specific manufacturing runs fail for various reasons, including not only general manufacturing failures but also specific failures due to the depleted state of the patient-donor's immune system following chemotherapy or radiation therapy. In addition, the expansion of αβ T cells (or the CAR-modified, “CAR-T” cells) in vivo can sometimes cause a large, rapid release of cytokines into the blood, resulting in CRS that can be severe or life-threatening.

Therapeutic immune cell compositions described herein therefore provide advantages over cell compositions that include a majority of adaptive immune cells. Therapeutic immune cell compositions described herein also are modified and altered relative to biological samples obtained from a subject that occur naturally. Therapeutic immune cell compositions, and methods of manufacture and therapeutic use, are described in detail hereafter.

### Methods of Manufacturing Innate Immune Cell Compositions

Provided herein are methods of manufacturing innate immune cell compositions. The compositions manufactured by the methods provided herein often contain a mixture of two activated populations of innate immune cells: the natural killer (NK) cells and the gamma.delta T cells.

The immune cell compositions obtained by the methods provided herein are enriched in NK cells and gamma.delta T cells, which are innate immune cells, relative to the composition of immune cells in nature (e.g., biological fluids and tissues). In nature, alpha.beta T cells, which are adaptive immune cells, are found in much higher amounts than NK cells and gamma.delta T cells. On the other hand, in the compositions provided herein, alpha.beta T cells are absent or present in negligible to low amounts, with NK cells and gamma.delta T cells being the predominant immune cell components.

For example, in human peripheral blood, it has been found that in the peripheral blood of healthy human donors: (i) for individuals under 40 years old, the median percentage of NK cells in PBMCs is 5% and the median percentage of T cells (combined CD8+ and CD4+ population, which represents alpha.beta T cells as gamma.delta T cells typically are CD8−CD4−) is 53%; and (ii) for individuals over 40 years old, the median percentage of NK cells in PBMCs is 10% and the median percentage of T cells (combined CD8+ and CD4+ population) is 37% (Lepone et al., J. Circ. Biomark., 5(5):1-17 (2016)). Thus, in peripheral blood, for every 1 circulating NK cell, there are approximately almost 4 circulating alpha.beta T cells in individuals greater than 40 years old, and for individuals under 40 years old, for every 1 circulating NK cell, there are approximately slightly over 10 circulating alpha.beta T cells. Therefore, the ratio of NK cells to alpha.beta T cells in circulating blood is in the range of about 1:4 to about 1:10. On the other hand, in the compositions provided herein, NK cells constitute 20% or more, up to 99% of the composition, while alpha.beta T cells are practically absent and constitute less than 2% of the composition, generally almost 0% or less than 0.5%, 0.4%, 0.3%, 0.2% or 0.1% of the composition. Thus, in the compositions provided herein, the ratio of NK cells to alpha.beta T cells is at least 10:1 and generally much higher than 10:1, e.g., about or greater than 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, or a 1000:1 or more. Thus, while alpha.beta T cells are predominant relative to NK cells in nature, the NK cell population is greatly increased relative to the alpha.beta T cell population in the compositions provided herein.

With respect to gamma.delta T cells, for example, in peripheral blood, less than about 10% of the T cells, generally about 5% of the T cells, are gamma.delta T cells while the rest are alpha.beta T cells (Esin et al., Scand. J. Immunol., 43(5):593-596 (1996); Radestad et al., J. Immunol. Res., Article ID 578741 (2014)). Therefore, the ratio of gamma.delta T cells to alpha.beta T cells in circulating blood is in the range of about 1:10 to about 1:20. On the other hand, in the compositions provided herein, gamma.delta T cells constitute 1% or more, generally between 2% or more up to 70-75% of the composition, while alpha.beta T cells are practically absent and constitute less than 2% of the composition, generally almost 0% or less than 0.5%, 0.4%, 0.3%, 0.2% or 0.1% of the composition. Thus, in the compositions provided herein, the ratio of gamma.delta T cells to alpha.beta T cells is at least 0.5:1, even assuming a composition in which gamma.delta T cells are present at 1% and alpha.beta T cells are present at 2%. Generally, the ratio of gamma.delta T cells to alpha.beta T cells in the compositions provided herein is much higher, e.g., about or greater than 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 150:1, 200:1, 250:1, 300:1, 350:1, 400:1, 450:1, 500:1, 550:1, 600:1, 650:1, 700:1, or 750:1 or more.

The term “enriched,” as used herein, means that the following two ratios: (i) gamma.delta T cells to alpha.beta T cells, and (ii) NK cells to alpha.beta T cells in the compositions provided herein are higher than these ratios in nature, e.g., in biological samples such as peripheral blood. In general, as used herein, “enriched” means that the ratio of gamma.delta T cells to alpha.beta T cells in the compositions provided herein is increased by at least 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, or 750-fold or higher, relative to the ratio in a biological sample, such as a tissue, cord blood or peripheral blood. With respect to NK cells, in general, as used herein, “enriched” means that the ratio of NK cells to alpha.beta T cells in the compositions provided herein is increased by at least 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold or higher, relative to the ratio in a biological sample, such as a tissue, cord blood or peripheral blood. In certain aspects, the term “enriched” means that the compositions provided herein have a ratio of NK cells to alpha.beta T cells and a ratio of gamma.delta T cells to alpha.beta T cells of greater than 1 (this ratio generally being less than 1 in nature).

In peripheral blood, B cells and alpha.beta T cells, which are adaptive immune cells, make up the majority of the lymphocytes. The compositions manufactured by the methods provided herein, on the other hand, are enriched in the innate immune cells, i.e., NK cells and gamma.delta T cells. The terms alpha.beta T cells and αβ T cells are used interchangeably herein to refer to the adaptive immune T cells, while the terms gamma.delta T cells and γδ T cells are used interchangeably herein to refer to the innate immune T cells.

Compositions for immunotherapy that are made up of innate immune cells are advantageous over those containing adaptive immune cells, for a variety of reasons. The innate immune response is non-specific and is initiated against a target of interest (e.g., cancer or infectious disease) within minutes to hours after infection. Innate immune cells can be expanded ex vivo. In the methods provided herein, the expansion conditions can result in compositions of NK cells and gamma.delta T cells that are not exhausted for as long as 60 days after expansion, which permits longevity of storage (amenability to off-shelf preservation and readministration as needed) and the ability to administer multiple doses cost-effectively. Adaptive immune cells, on the other hand, initiate an antigen-specific immune response and expand upon binding to the target antigen in vivo, which can take days or even weeks. Further, unlike innate immunity, the adaptive immune response relies on HLA antigens, which requires the cells to be autologous or “matched” to the recipient patient/subject to minimize or avoid GvHD (graft vs. host disease). Compositions containing innate immune cells, such as the NK cells and gamma.delta (γδ) T cells in the compositions made by the methods provided herein, do not require HLA antigen recognition to mediate killing and, therefore can be used in a more widespread off-the-shelf (allogeneic) setting, while mitigating GvHD. Adaptive immune cells also produce large amounts of cytokines upon expansion in vivo, which can lead to cytokine release syndrome (CRS); innate immune cells can be expanded ex vivo, thereby minimizing or avoiding CRS.

In the methods provided herein, a sample containing cells, such as peripheral blood or cord blood, is obtained from a subject. The subject generally is a healthy donor but can also be a patient in need of treatment with an immunotherapy composition made by a method provided herein. In certain aspects, when the sample is peripheral blood, peripheral blood universal donor banks can be used as a source of the sample. The sample sometimes is subjected to activation conditions that include contacting the sample with: (a) at least one exogenous polypeptide that immunospecifically binds to a cell adhesion polypeptide, and (b) at least one exogenous polypeptide that immunospecifically binds to a different polypeptide than the cell adhesion polypeptide and is expressed on the surface of one or more cells of the sample population. Examples of cell adhesion polypeptides include, but are not limited to, CD2 (see, e.g., GenBank Accession Nos. KJ905161.1; KJ896558.1), LFA-1 (see, e.g., GenBank Accession No. BC005861.2), LFA-3 (see, e.g., GenBank Accession No. BC005930.1), CD8 (see, e.g., GenBank Accession Nos. AH003215.2; AY039664.1 for CD8A; see, e.g., GenBank Accession Nos. KJ896562.1; BC100911.1; BC100912.2; BC100913.1; BC100914.1 for CD8B) and CD4 (see, e.g., GenBank Accession Nos. M35160.1; DQ892052.2). Examples of polypeptides that are expressed on the surface of one or more cells of the sample population, such as NK cells and/or gamma.delta cells, include, but are not limited to, CD2 (see, e.g., GenBank Accession Nos. KJ905161.1; KJ896558.1), CD3 (see, e.g., GenBank Accession No. AB583162.1 for CD3y; see, e.g., GenBank Accession No. AB583139.1 for CD3s; see, e.g., GenBank Accession No. AH002612.2 for CD36), CD56 (see, e.g., GenBank Accession Nos. U63041.1; BC047244.1; BC029119.1), NKp30 (see, e.g., GenBank Accession No. AB055881.1), NKp44 (see, e.g., GenBank Accession No. BC166647.1), NKp46 (see, e.g., GenBank Accession No. BC064806.1; AY346373.1), NKG2A (see, e.g., GenBank Accession Nos. AF461812.1; BC053840.1), PD-1 (see, e.g., GenBank Accession No. L27440.1), NKG2C (see, e.g., GenBank Accession Nos. BC093644.1; BC112039.1), NKG2D (see, e.g., GenBank Accession Nos. AF461811.1; BC039836.1), KAR receptors, KIR receptors, SIGLEC-7 (see, e.g., GenBank Accession Nos. AF193441.1; AF170485.1), KIR3DS1 (see, e.g., GenBank Accession No. EU156175.1), KIR2DL1 (see, e.g., GenBank Accession Nos. LT984790.1; LT984791.1; antibody: 11PB6), DNAM1 (see, e.g., GenBank Accession Nos. BC074787.2; U56102.1), NTBA (see, e.g., GenBank Accession Nos. BC114495.1; BC113893.1), HLA-DR (see, e.g., GenBank Accession No. AH001506.2 for α; AH002824.2 for β) and the like. Activation of the cells generally initiates an innate immune response in the cells of the compositions provided herein, which can be used to target a disease, such as a cancer or infectious disease, in a subject in need of treatment for such a disease.

A polypeptide immunospecifically binds a region of another molecule (i.e., an epitope) if it reacts or associates more frequently, more rapidly, with greater duration and/or with greater affinity with that epitope relative to alternative epitopes. For example, a polypeptide (e.g., an antibody or antigen-binding fragment thereof) that immunospecifically binds to a first epitope is an antibody that binds this first epitope with greater affinity, avidity, more readily, and/or with greater duration than it binds to a second epitope or other epitopes. A polypeptide that immunospecifically binds to a first target may or may not specifically or preferentially bind to a second target, and immunospecific binding is not necessarily exclusive binding.

In certain aspects of the methods provided herein, the activation conditions include contacting the sample or with at least two exogenous polypeptides. In some aspects, the first exogenous polypeptide immunospecifically binds to CD2 and the second exogenous polypeptide immunospecifically binds to NKp46. In some aspects, the activation conditions consist or consist essentially of a first exogenous polypeptide that immunospecifically binds to CD2 and a second exogenous polypeptide that immunospecifically binds to NKp46. The phrase, “consist(s) essentially of,” as used herein, means that components other than the recited components, if present, do not materially alter the activity of the recited components. Thus, for example, in the context of the aforementioned activation conditions, the activation conditions may include one or more components other than the first exogenous polypeptide that immunospecifically binds to CD2 and the second exogenous polypeptide that immunospecifically binds to NKp46 that do not materially alter the activity of the first exogenous polypeptide and the second exogenous polypeptide. In certain aspects, the first exogenous polypeptide and/or the second exogenous polypeptide is/are an antibody or an antigen-binding fragment thereof.

The sample also often is subjected to expansion conditions that includes contacting the sample with at least one supplemental polypeptide, thereby generating a composition comprising a population of cells enriched in NK cells and gamma.delta T cells. In certain aspects of the methods provided herein, the expansion conditions include at least one supplemental polypeptide that is a cytokine and/or a polypeptide that immunospecifically binds to a receptor on a gamma.delta T cell. In certain aspects, the cytokine is an interleukin such as, for example, IL-1, IL-2, IL-4, IL-7, IL-9, IL-15, IL-21 or any combinations thereof. In some aspects of the methods provided herein, the cytokine is IL-2, IL-15 or a combination thereof. In aspects, the expansion conditions include, consist of or consist essentially of IL-2, IL-15 and a polypeptide that immunospecifically binds to a receptor on a gamma.delta T cell. In certain aspects, the receptor on the gamma.delta T cell is CD3. In some aspects, the polypeptide that immunospecifically binds to the CD3 receptor on the gamma.delta T cell is an antibody or an antigen-binding fragment thereof and, in certain aspects, the antibody is OKT3. In certain aspects, the expansion conditions include, consist of or consist essentially of, contacting the sample with: (a) an IL-2 polypeptide; (b) an IL-15 polypeptide; (c) an IL-2 polypeptide and an IL-15 polypeptide; (d) an IL-2 polypeptide and an antibody that immunospecifically binds CD3; or (e) an IL-2 polypeptide, an IL-15 polypeptide and an antibody that immunospecifically binds CD3. In some aspects, the antibody that immunospecifically binds CD3 is OKT3.

In some aspects, the activation conditions consist or consist essentially of contacting the sample with a first exogenous polypeptide that immunospecifically binds to CD2 and a second exogenous polypeptide that immunospecifically binds to NKp46, and the expansion conditions consist or consist essentially of contacting the sample with: (a) an IL-2 polypeptide; (b) an IL-15 polypeptide; (c) an IL-2 polypeptide and an IL-15 polypeptide; (d) an IL-2 polypeptide and an antibody that immunospecifically binds CD3; or (e) an IL-2 polypeptide, an IL-15 polypeptide and an antibody that immunospecifically binds CD3. In some aspects, the antibody that immunospecifically binds CD3 is OKT3.

In certain aspects, the methods provided herein for manufacturing compositions include depleting alpha.beta T cells from the sample, prior to activation and expansion, thereby generating a depleted cell population that then can undergo activation and expansion. For example, the alpha.beta T cells can be depleted from the sample using an antibody that immunospecifically binds to an alpha.beta T cell receptor, e.g., alpha.beta TCR. The term “depleted,” as used herein, means that substantially all of the depleted component (e.g., alpha.beta T cells) has been removed from the sample, for example, at least about 80%, at least about 85%, at least about 90%, at least about 95%, at least about 96%, at least about 97%, at least about 98%, at least about 99% or greater, up to about 100% of the depleted component has been removed from the sample. In general, the term “depleted cell population,” as used herein, refers to the population of cells that is derived from the sample after depleting the alpha.beta T cells from the sample. In certain aspects, the sample additionally is depleted of B cells using, for example, a polypeptide that immunospecifically binds to a B cell receptor, e.g., CD19.

For example, when the sample is a peripheral blood sample, the sample can be processed by density gradient centrifugation to separate and/or isolate a buffy coat containing white blood cells, platelets, granulocytes and the like, which then can be subjected to alpha.beta T cell depletion according to the methods provided herein. In certain aspects, the buffy coat can further undergo a Ficoll gradient separation to obtain mononuclear cells (PBMCs), which then can be subjected to alpha.beta T cell depletion in the methods provided herein. In some aspects, the peripheral blood sample can undergo apheresis to separate the plasma from the cells (e.g., using a Terumo Optia machine) and, in certain aspects, the cells then are subjected to alpha.beta T cell depletion in the methods provided herein. Alpha.beta T cell depletion can be performed by methods known to those of skill in the art. In certain aspects, the alpha.beta T cells can be depleted using a Miltenyi LS column. In some aspects, the apheresis product can undergo alpha.beta T cell depletion using the Miltenyi Clinimacs separation device. The depleted cell population can, if needed, or desired, be cryopreserved (e.g, at negative 70, 75, 80, 85 or lower degrees Celsius) and stored prior to activation and expansion. Non-limiting examples of culture conditions for activation and expansion include activation and expansion conditions described in the Examples section herein for cell populations containing NK cells and gamma.delta cells or NK MACS™ Medium (#130-107-879 (Miltenyi Biotec, Inc., San Diego, Calif., USA) supplemented with 5% AB serum, or other media used in the art (e.g., R&D Systems, CellGenix).

In certain aspects, a cell population is not exposed to conditions that positively select for NK cells or positively select for gamma.delta T cells prior to activation and expansion conditions described herein.

In certain aspects of the methods provided herein, the sample or the depleted cell population is subjected to activation conditions in which at the exogenous polypeptide in (a), the exogenous polypeptide in (b), or both the exogenous polypeptide in (a) and the exogenous polypeptide in (b) are soluble. In aspects, the soluble exogenous polypeptide in (a), the soluble exogenous polypeptide in (b), or the soluble exogenous polypeptides in both (a) and (b), is/are an antibody or antigen-binding fragment thereof. The term “soluble” as used herein in reference to a component, such as a polypeptide, means that the component is not bound to a solid phase or support and is in a homogeneous single phase or an emulsion in the culture conditions (e.g., activation conditions, expansion conditions). In some aspects, the exogenous polypeptide in (a) or (b), e.g., and antibody, is bound to a solid phase or support. Examples of solid supports include, but are not limited to, silica, glass (e.g. glass, controlled-pore glass (CPG)), nylon, Wang resin, Merrifield resin, Sephadex, Sepharose, cellulose, magnetic beads, Dynabeads, a metal surface (e.g. steel, gold, silver, aluminum, silicon and copper), a plastic material (e.g., polyethylene, polypropylene, polyamide, polyester, polyvinylidenedifluoride (PVDF)). The solid support can be in any desired form, including, but not limited to: a bead, chip, capillary, plate, membrane, wafer, comb, pin, a substantially flat surface, an array of pits or nanoliter wells and other geometries and forms known to those of skill in the art. The use of soluble exogenous polypeptides rather than polypeptides bound to solid supports can mitigate steric hindrance while increasing scalability of the method (e.g., for GMP manufacturing). In certain aspects, the first exogenous polypeptide is a soluble anti-CD2 antibody and the second polypeptide is a soluble anti-NKp46 antibody. In certain aspects, either the anti-CD2 antibody or the anti-NKp46 antibody is bound to a solid support.

Antibodies, such as polyclonal antibodies and monoclonal antibodies, can be prepared using standard methods (see, e.g., Kohler et al., Nature 256:495-497 (1975); Kohler et al., Eur. J. Immunol. 6:511-519 (1976); and WO 02/46455). For example, to generate polyclonal antibodies, an immune response is elicited in a host animal, to an antigen of interest. Blood from the host animal is then collected and the serum fraction containing the secreted antibodies is separated from the cellular fraction, using methods known to those of skill in the art. To generate monoclonal antibodies, an animal is immunized by standard methods to produce antibody-secreting somatic cells. These cells then are removed from the immunized animal for fusion to myeloma cells. Somatic cells that can produce antibodies, particularly B cells, can be used for fusion with a myeloma cell line. These somatic cells can be derived from the lymph nodes, spleens and peripheral blood of primed animals. Specialized myeloma cell lines have been developed from lymphocytic tumors for use in hybridoma-producing fusion procedures (Kohler and Milstein, Eur. J. Immunol. 6:511-519 (1976); Shulman et al., Nature, 276:269-282 (1978); Volk et al., J. Virol., 42:220-227 (1982)). These cell lines have three useful properties. The first is they facilitate the selection of fused hybridomas from unfused and similarly indefinitely self-propagating myeloma cells by having enzyme deficiencies that render them incapable of growing in selective medium that support the growth of hybridomas. The second is they have the ability to produce antibodies and are incapable of producing endogenous light or heavy immunoglobulin chains. A third property is they efficiently fuse with other cells. Other methods for producing hybridomas and monoclonal antibodies are well known to those of skill in the art. It is routine to produce antibodies against any polypeptide, e.g., antigenic marker on an immune cell population.

In certain aspects of the methods provided herein, the activation conditions, expansion conditions, or both the activation and expansion conditions are feeder cell free. The term “free,” as used herein (e.g., free of feeder cells or feeder cell free, serum free, free of serum from a non-human animal, free of exhausted cells) means that the conditions are substantially free, i.e., at least 80%, 85%, 90%, 95%, generally 95% or more, e.g., 96%, 97%, 98%, 99% or more, up to 100% free of the component (i.e., feeder cells, serum, exhausted cells or other components as referred to herein). In certain aspects of the methods provided herein, the sample, the activation conditions, the expansion conditions, or the activation and expansion conditions, or all of the foregoing, are free of exogenous cells, free of exogenous feeder cells, free of irradiated cells and/or free of irradiated feeder cells. Exogenous cells and exogenous feeder cells generally are cells from a different subject, or cells from a different portion of the subject, compared to the subject or the portion of the subject from which sample cells were obtained and subjected to activation and/or expansion conditions. In non-limiting examples, (i) sample cells are from a subject of a first species and exogenous cells are from a subject of a second species (e.g., the sample cells are from a human and the exogenous cells are from a non-human animal such as a rodent or monkey), and (ii) sample cells are from peripheral blood of a human subject, and exogenous cells are from a different portion of the same subject (e.g., from cord blood or an organ of the same subject or are from a different human subject). A composition generally is “free” of a certain component when a sample is not contacted with that component during one or all of the following: processing prior to activation and/or expansion, activation and expansion.

Reliance on feeder cells, such as K562 cells or others, can limit where and how the cells are cultured, and can significantly increase the cost of culturing the cells. The use of feeder cells also can be problematic due to cell culture variability caused by undefined biological factors derived from the feeder cells. In addition, feeder cells have the potential to introduce unwanted agents (e.g., retroviruses, other pathogens, and immunogenic nonhuman sialic acid such as Neu5Gc) into the compositions made by the methods provided herein, which can be undesirable for certain applications such as, for example, transplantation. Without being bound by a theory, in the methods provided herein, the exogenous polypeptide that immunospecifically binds to a cell adhesion polypeptide may generate an “endogenous-feeder cell-like layer”, thereby generating a significantly activated expanded cell population without the need for exogenous feeder cells.

In certain aspects of the methods provided herein, the choice of the supplemental polypeptide(s) and/or the expansion times in the expansion conditions can be tailored to the desirable relative amounts of NK cells and gamma.delta T cells. For example, compositions that contain relatively more NK cells and relatively less gamma.delta T cells in general find greater applicability against solid tumors, while compositions that contain relatively more gamma.delta T cells and relatively less NK cells in general find greater applicability against hematologic malignancies. The supplemental polypeptide(s) can be selected and/or the expansion reactions can be performed for times that facilitate obtaining compositions with the desired proportions of NK cells and gamma.delta T cells. A desired proportion of NK cells relative to gamma.delta T cells can be obtained by selection of particular expansion conditions, and non-limiting examples of combinations follow: (i) when the supplemental polypeptide is IL-2, the resulting population of cells enriched in NK cells and gamma.delta T cells often contains about 25-30% NK cells and about 70-75% gamma.delta T cells; (ii) when the supplemental polypeptide is IL-15, the resulting population of cells enriched in NK cells and gamma.delta T cells often contains about 80-99% NK cells and about 1-20% gamma.delta T cells; (iii) when the supplemental polypeptide is IL-2 and an antibody that immunospecifically binds CD3, such as OKT3, the resulting population of cells enriched in NK cells and gamma.delta T cells often contains about 40-45% NK cells and about 55-60% gamma.delta T cells; (iv) when the supplemental polypeptide is IL-2 until Day 20 of the expansion conditions and then switched to IL-15 until Day 30, compared to treatment with IL-2 alone, the percentage of gamma.delta T cells often increases from about 50% to about 70%, with a corresponding decrease in the percentage of NK cells; and (v) when the supplemental polypeptide is IL-15 until Day 20 of the expansion conditions and then switched to IL-2 until Day 30, compared to treatment with IL-15 alone, the percentage of NK cells often increases from about 80% to about 90%, with a corresponding decrease in the percentage of gamma.delta T cells.

Duration of expansion conditions (for example, 12 days vs. 25 days) also can control the relative amounts of NK cells and gamma.delta T cells in the compositions made by the methods provided herein. For example, the length of incubation of the sample or depleted cell population in a feeder cell free medium under expansion conditions can be for about 7, 8, 9, 10, 11, 12, 13, 14, 15, 16, 17, 18, 19, 20, 21, 25, 30, 35, 40, 45, 50, 55 or 60 or more days or about 2, 3, 4, 5, 6, 7, 8, 9 or 10 weeks. In certain aspects of the methods provided herein, the compositions are free of exhausted cells, and, in some aspects, the compositions are free of exhausted cells after at least 60 days of expansion conditions. Preparing cell compositions substantially free of exhausted cells permits immediate treatment and multiple dosing at a fraction of the cost of many current immunotherapies (e.g., CAR-T using alpha.beta T cells), due to the increased ex vivo availability of expanded, cytotoxic cell compositions.

In certain aspects of the methods provided herein, the activation, or expansion, or activation and expansion conditions do not include bisphosphonates, resulting in a polyclonal population of V.delta.1 and V.delta.2 gamma.delta T cells. The use of bisphonates, such as zoledronate and pamidronate can favor one clonal population of gamma.delta T cells over the other, which can limit the repertoire of the compositions against certain tumors and infections. In some aspects, the absence of bisphosphonates in the methods provided herein generates compositions that are polyclonal with respect to the gamma.delta T cells, thereby increasing the range of cancers and infectious diseases for which they can be administered as immunotherapy.

A non-limiting example of an outline of steps for conducting a method provided herein follows:

(1) A sample from a donor (e.g., peripheral blood directly from a donor or from a blood bank) is thawed, if needed, and placed in culture; (2) if desired, the culture is subjected to alpha.beta T cell depletion and, optionally, B cell depletion; (3) the sample or the remaining cells after depletion (depleted cell population) are subjected to activation conditions for 3-4 days, generating activated cells; (4) if desired, the activated cells are transduced with an exogenous polynucleotide using, for example, a retroviral vector or a lentiviral vector; (5) after activation in (3) or transduction in (4), the cells are subjected to expansion conditions in 7 day cycles, with washes between cycles, generally for 2-3 cycles, thereby generating expanded cells, which sometimes are used immediately or at other times refrigerated, maintained on ice or cryopreserved for transportation and/or storage until needed for immunotherapy. The proportions of NK cells and gamma.delta T cells can be adjusted by the choice of supplemental polypeptide(s) used during expansion, including switching supplemental polypeptides between 7 day cycles, if desired, and by controlling the expansion times.

In certain aspects, a composition resulting from a sample cell population being exposed to activation and expansion conditions has the following characteristics:
(i) a ratio of NK cells to gamma.delta T cells of greater than 1 (e.g., a ratio of about 1.5 or greater, 2 or greater, 3 or greater, 4 or greater, or 5 or greater; a ratio of about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.2, 4.4, 4.6, 4.8, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 40, 50, 60, 70, 80, 90 or 100 or greater); or
(ii) a ratio of NK cells to gamma-delta T cells of less than 1 (e.g., a ratio of about 0.8 or less, 0.7 or less, 0.6 or less, 0.5 or less, 0.1 or less, 0.05 or less, 0.01 or less; a ratio of about 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001 or less);
and, optionally, has one or more of the following characteristics:
(iii) a ratio of NK cells to alpha.beta T cells of greater than 2, 3, 4, 5, 6, 7, 8, 9, 10, 20, 30, 40, 50, 60, 70, 80, 90, 100, 200, 300, 400, 500, 600, 700, 800, 900, 1000 or greater;
(iv) a ratio of gamma-delta T cells to alpha.beta T cells of greater than 2, 3, 4, 5, 6, 7, 8, 9, 10, 20, 30, 40, 50, 60, 70, 80, 90, 100, 200, 300, 400, 500, 600, 700, 800, 900, 1000 or greater;
(v) a percentage of NK cells relative to total cells of 20% or greater;
(vi) a percentage of gamma.delta T cells relative to total cells of 2%, 3%, 4%, 5% or greater;
(vii) about 75% to about 95% of the gamma.delta T cells (e.g., about 80% to about 90% (e.g., about 85%) of the gamma.delta T cells) express V.delta.1;
(viii) about 10% to about 25% of the gamma.delta T cells (e.g., about 10% to about 20% (e.g., about 15% of the gamma.delta T cells) express V. delta.2;
(ix) about 30% to about 60% of the gamma.delta T cells (about 35% to about 55% of the gamma.delta T cells) express V.delta.1;
(x) about 35% to about 60% of the gamma.delta T cells (e.g., about 40% to about 50% or 55% of the gamma.delta T cells) express V.delta.2;
(xi) about 10% to about 30% of the gamma.delta T cells (e.g., about 20% to about 25% of the gamma.delta T cells) express V.delta.1;
(xii) about 65% to about 80% of the gamma.delta T cells (e.g., about 70% to about 80% (e.g., about 75% of the gamma.delta T cells) express V.delta.2;
(xiii) about 80% or more of the total cells (e.g., about 90% or more of the cells) express KIR5;
(xiv) about 5% or more of the total cells (e.g., about 10% or more of the cells) express SIGLEC-7;
(xv) about 50% or more of the total cells (e.g., about 60% or more of the cells) express KIR3D51;
(xvi) about 5% or more of the total cells (e.g., about 10% or more of the cells) express KIR2DL1;
(xvii) about 20% or more of the total cells (e.g., about 25% or more of the cells) express NKp30, NKp44 or NKp46;
(xviii) about 25% or more of the total cells (e.g., 35% or more of the cells express) NKG2D; (xix) about 80% or more of the total cells (e.g., about 90% or more of the cells) express DNAM1;
(xx) about 75% or more of the total cells (e.g., about 85% or more of the cells) express NTBA;
(xxi) about 85% or more of the total cells (e.g., about 95% or more of the cells) express CD2;
(xxii) about 80% to about 100% of the total cells (e.g., at least about 81%, 82%, 83%, 84%, 85%, 86%, 87%, 88%, 89%, 90%, 91%, 92%, 93%, 94%, 95%, 96%, 97%, 98%, 99% or 100% of the cells) express CD56;
(xxiii) about 51% to about 100% of the total cells (e.g., at least about 51%, 52%, 53%, 54%, 55%, 56%, 57%, 58%, 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% of the cells) do not express CD57;
(xxiv) about 10% to about 40% of the total cells (e.g., at least about 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the cells) express CD16;
(xxv) about 10% or less of the total cells (e.g., about 5% or less, 4% or less, 3% or less, 2% or less of the cells) express CD57;
(xxvi) about 50% or more of the total cells (e.g., about 55% or more of the cells) express KIR3DS1;
(xxvii) about 5% or less of the total cells (e.g., about 4% or less, 3% or less, 2% or less, 1% or less, 0.9% or less, 0.8% or less, 0.7% or less, 0.6% or less, 0.5% or less, 0.4% or less, 0.3% or less, 0.2% or less, 0.1% or less) are NKT cells;
(xxviii) about 5% or less of the total cells (e.g., about 4% or less, 3% or less, 2% or less, 1% or less, 0.9% or less, 0.8% or less, 0.7% or less, 0.6% or less, 0.5% or less, 0.4% or less, 0.3% or less, 0.2% or less, 0.1% or less) are alpha.beta T cells;
(xxix) about 50% or more of NK cells and/or gamma.delta cells (e.g., about 51%, 52%, 53%, 54%, 55%, 56%, 57%, 58%, 59%, 60%, 6%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99% 80%, 81%, 82%, 83%, 84%, 85%, 86%, 87%, 88%, 89%, 90%, 91%, 92%, 93%, 94%, 95%, 96%, 97%, 98%, 99% or fractions thereof, up to 100%, of the NK cells and/or gamma.delta T cells) express CD8;
(xxx) about 5% or less of NK cells and/or gamma.delta cells (e.g., about 4% or less, about 3% or less, about 2% or less or about 1% or less of NK cells and/or gamma.delta T cells) express CD4;
(xxxi) about 5% or less of NK cells and/or gamma.delta cells (e.g., about 4% or less, about 3% or less, about 2% or less or about 1% or less of NK cells and/or gamma.delta T cells) express CD8 and CD4;
(xxxii) about 15% to about 35% of NK cells (e.g., 16%, 17%, 18%, 19%, 20%, 22%, 24%, 26%, 28%, 30%, 32%, 34% of NK cells) do not express CD8 and CD4; and
(xxxiii) about 55% to about 85% of gamma.delta T cells (e.g., about 56%, 58%, 60%, 62%, 63%, 66%, 68%, 70%, 72%, 74%, 76%, 78%, 80%, 82%, 84% of gamma.delta T cells) do not express CD8 or CD4.

### Innate Immune Cell Compositions

Certain compositions prepared by methods of manufacture described herein are referred to BINATE compositions. The BINATE compositions contain two innate cell types, NK cells and gamma.delta T cells, and the relative amounts of these two cell types can be adjusted to treat solid tumors, hematologic cancers or infectious diseases, by selecting a suitable supplemental polypeptide or polypeptides, including the order of their use and the duration of the expansion conditions.

In some aspects, the BINATE compositions do not contain feeder cells and often these compositions are free of feeder cells (e.g., free of exogenous feeder cells and/or irradiated feeder cells). In certain aspects, the activation and expansion conditions do not include bisphosphonates, thereby rendering polyclonal gamma.delta T cells that have a wider repertoire of activity against tumors and infectious diseases (i.e., polyclonal with respect to V.delta.1 and V.delta.2 gamma.delta T cells). This innate immune cell platform, interchangeably termed BINATE cells, INNATE cells or INNATE-K cells herein, can comprehensively engage both tumor signals and receptors and provides readily available, universal therapy for solid tumors, blood cancers and infections. The improved safety of innate immunotherapies can allow for use at community hospitals, reducing medical costs and bringing effective treatments to more patients.

In certain aspects, the BINATE cell compositions provided herein can further be treated to generate compositions containing substantially all NK cells (termed “INNATE-NK” herein) or substantially all gamma.delta T cells, using markers that either affirmatively select for a cell population of interest or that eliminate an undesired cell population.

The BINATE compositions provided herein can be developed as: a) non-gene modified cells in the post-transplant or other settings/indications; b) non-gene modified cells dosed in combination with other therapeutic agents, e.g., commercially available antibodies for cancer treatment; or c) genetically modified cells, e.g., by mutating an endogenous polynucleotide, by deleting an endogenous polynucleotide or by adding exogenous mutated polynucleotide (where the wild type form is in the unmodified cell) or adding an exogenous polynucleotide of a heterologous nature, e.g., a CAR polynucleotide (BINATE.CAR) for targeting both solid tumor and hematological malignancies. The CAR modified INNATE-K (same as BINATE), or INNATE-NK populations are designated herein by any of the following terms:

For immune cells modified by a CAR in general, the cells are referred to with a “CAR” suffix preceded by a period or a hyphen, e.g., INNATE-CAR, INNATE-K.CAR, INNATE-NK.CAR or BINATE.CAR; and

For immune cells modified by a specific CAR, e.g., a CAR targeted to CD19, the cells are referred to interchangeably with a “CD19,” a “CAR19” or a “CAR.CD19” suffix preceded by a period or a hyphen, e.g., BINATE.CD19, BINATE.CAR19 or BINATE.CAR.CD19.

The methods of manufacture provided herein result in a BINATE population of cells having a high level of activation of between about 30% to about 40%, 50%, 60%, 70%, 75%, 80%, 85%, 90% 95% or more of the cells in the population, or at least about 30% 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or more, up to 100% of the cells in the population. In comparison, triggering the innate immune response in vivo results in 10% or less of the cells becoming activated.

These activated cells are highly cytotoxic, with a significant proportion of CD56+CD16+ cells (e.g., about 40% of the NK cell population in the compositions provided herein can be CD56+CD16+). As used herein, the “+” symbol or the word “positive” in reference to the description of cellular markers on a cell indicates that the marker is expressed in the cell (or on the cell surface), whereas the “−” symbol or the word “negative” in reference to the description of cellular markers on a cell indicates that the marker is not present or detected.

While the cytotoxicities of the BINATE cells of the compositions provided herein are high, their maturity is low (i.e., they are farther away from senescence) because the CD57 marker levels of the majority of the cells are low (high CD57 marker levels is indicative of cytoxicity but also is indicative of the cells being closer to senescence and, therefore, having a shorter lifetime (see Kared et al., Cancer Immunol. Immunotherap., 65(4):441-452 (2016)). The term “majority,” as used herein, means greater than 50%, generally 50.5% or more, for example, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or fractions thereof, to up to 100% of the cells in the population. The BINATE cell compositions made by the methods of manufacture provided herein also have low levels of exhaustion markers, such as PD-1 and TIM-3. Non-limiting examples of activation/cytotoxicity/exhaustion marker phenotypes for the BINATE compositions obtained under different expansion conditions according to the methods of manufacture provided herein are summarized below:

| | NK CELLS | | | TCRgd (gamma.delta T) CELLS | | |
|---|---|---|---|---|---|---|
| Expansion Conditions | IL-2 | IL-2 + OKT3 | IL-15 | IL-2 | IL-2 + OKT3 | IL-15 |
| % NK or gamma.delta T cells | 27.25% | 42.50% | 98.75% | 72.27% | 56.61% | 1.23% |
| Markers | | | | | | |
| CD16 | 51.21 | 65.17 | 40.88 | 18.64 | 29.12 | 28.87 |
| KIRs | 98.47 | 96.25 | 93.37 | 96.43 | 92.18 | 100 |
| NKG2A | 98.43 | 94.09 | 99.04 | 94.81 | 97.06 | 93.81 |
| LIR-1 | 34.54 | 49.14 | 10.14 | 64.02 | 50.41 | 19.59 |
| CD57 | 54.99 | 41.96 | 2.46 | 69.14 | 46.23 | 35.05 |
| NKG2C | 13.46 | 23.76 | 2.18 | 3.46 | 2.67 | 12.37 |
| p75 (indirect APC-Cy7) inhibitor SIGLEC-7 | 72.81 | 22.85 | 97.42 | 6.19 | 1.97 | 56.19 |
| KIR3DS1-activator | 57.03 | 68.08 | 69.11 | 62.4 | 65.4 | 69.3 |
| KIR3DL1 (direct FITC) inhibitor | 11.7 | 13.24 | 22.62 | 3.99 | 2.99 | 18.52 |
| KIR2DL1 (direct APC)-activator | 38.11 | 47.73 | 10.14 | 27.41 | 37.1 | 15.79 |
| 11PB6 (direct Viob) KIR2DL1-inh KIR2DS1- | 24.83 | 38.73 | 7.43 | 9.22 | 4.35 | 20.37 |
| act | | | | | | |
| CH-LEO (indirect APC-Cy7) KIR2DL2 | 37.94 | 57.38 | 35.25 | 23.96 | 60.13 | 25.44 |
| KIR2DL3 KIR2DS2 | | | | | | |
| AZ20 (indirect PE) NKP30 | 83.62 | 78.52 | 99.9 | 10.37 | 7.6 | 54.17 |
| BAB281 (indirect PE) NKP46 | 84.45 | 77.7 | 99.86 | 12.96 | 11.72 | 19.48 |
| z231 (indirect PE) NKP44 | 67.01 | 60.14 | 99.58 | 19.74 | 6.25 | 78.35 |
| ECM217 (indirect APC-Cy7) NKG2D | 34.42 | 48.67 | 62.13 | 40.26 | 54.08 | 86.46 |
| GN18 (indirect FITC) DNAM1 | 90.54 | 95.66 | 94.38 | 96.68 | 99.77 | 96.91 |
| ST39 (indirect FITC) D24 | 99.19 | 96.3 | 99.76 | 97.26 | 98.17 | 96.88 |
| ON56 (indirect APC-Cy7) NTBA | 95.06 | 75.94 | 98.47 | 99.75 | 95.84 | 98.7 |
| EA4 (indirect FITC) LSF1 | 99.53 | 95.56 | 94.34 | 99.08 | 99.55 | 98.7 |
| TIGIT (indirect APC-Cy7) | 26.71 | 28.21 | 18.96 | 19.26 | 25.07 | 15.31 |
| D1.12 (indirect FITC) HLA-DR | 68.42 | 66.16 | 6.25 | 97.16 | 97.67 | 52.04 |
| TIM-3 (indirect APC) | 0.37 | 0.31 | 0.14 | 0.58 | 0.61 | 5.1 |
| QA196 (indirect APC) CD2 | 99.82 | 99.72 | 98.53 | 99.08 | 100 | 100 |
| CD8 (indirect APC-Cy7) | 58.16 | 68.02 | 91.17 | 45.98 | 74.48 | 97.94 |

In certain aspects, the cells of the BINATE compositions provided herein can genetically be modified. Certain non-limiting examples of genetic modifications include (i) adding an exogenous polynucleotide that encodes a polypeptide having a desired activity, (ii) altering or adding an endogenous polynucleotide or adding an exogenous regulatory polynucleotide (e.g., primer or enhancer) that regulates the expression of an endogenous polypeptide having the desired activity; (iii) altering and/or disrupting an endogenous polynucleotide that encodes a polypeptide having the desired activity (e.g., insertional mutagenesis), (iv) partially or completely deleting a regulatory polynucleotide that regulates the expression of a polypeptide having the desired activity, thereby disrupting its regulation, and/or (v) partially or completely deleting the coding sequence that encodes a polypeptide having the desired activity, whereby the activity is attenuated or abolished (e.g., knock out mutagenesis).

In some aspects, the BINATE composition is genetically modified by adding an exogenous (regulatory or coding sequence) polynucleotide. The highly activated cells of the BINATE composition can be transduced with high efficiency, often 80% or greater, generally at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or fractions thereof, to up to 100% of the cells in the population. This transduction rate is much higher than the rate often seen with adaptive immune cells (e.g., about 9% in alpha.beta T cells).

In certain aspects, the exogenous polynucleotide encodes a chimeric antigen receptor (CAR) and the cells in the composition comprise a CAR (referred to as “BINATE.CAR cells”). In some aspects, the CAR contains a binding molecule portion that immunospecifically binds to one or more of CD19, GD2, HER3, B7H3, CD123 or CD30.

The BINATE cells and BINATE.CAR cells provided herein can be used to target a variety of cancers and infectious diseases (e.g., GD2/HER3/B7H3: Lung/Bronchus, Prostate, Breast, Colon, Pancreas, Ovary; CD123: Leukemia; CD30: Non-Hodgkin Lymphoma; other cancers including Liver & Intrahepatic Bile Duct, Esophagus, Urinary Bladder, Kidney & Renal Pelvis, Uterine, Brain/Nervous System). GD2, a disialoganglioside, also is found expressed on the surface of tumor cells of neuroectodermal origin. Tumors with GD2 expression have a high mortality rate (Pediatric tumors—Neuroblastoma, Retinoblastoma, Sarcomas; Adult tumors—Melanoma, Non-Small Cell Lung, Breast). The monoclonal antibody currently being tested as a therapy has limitations due to toxicity of neuropathic pain. Initial studies with a BINATE.CAR.GD2 construct indicate equivalent high levels of transduction in both NK cells and gamma.delta T cells (about 80% with NK cells, about 40-60% with gamma.delta T cells); the expression is stable and maintained over the BINATE culture period. In addition, in vitro killing of solid tumor cells was observed (Example 18).

### Pharmaceutical Compositions and Kits

Any of the compositions provided herein can be formulated as a pharmaceutical composition in conjunction with a pharmaceutically acceptable carrier. Like the compositions, the pharmaceutical compositions provided herein can be used for treating cancers and infectious diseases. Also provided herein are kits containing the compositions or pharmaceutical compositions provided herein and, optionally, instructions for use. In certain aspects, the kits provided herein can include a cytokine. Innate immune cells control opportunistic invasion by a wide range of viral, fungal, bacterial, and parasitic pathogens, in part by releasing a plethora of cytokines and chemokines to communicate with other cells and thereby to orchestrate immune responses.

A pharmaceutical composition or kit sometimes includes specific dosage of therapeutic cells, and sometimes the pharmaceutical composition or kit provides a unit dosage of therapeutic cells. In certain aspects, a unit dosage is about 10⁴ to about 10¹⁰ cells per kilogram of weight of an intended subject, or between about 10° to about 10¹² cells per subject (e.g., about 10¹⁰ cells per subject or about 10⁸ cells per kilogram of weight of the intended subject).

A pharmaceutical composition or kit can include a pharmaceutically acceptable carrier. The term “pharmaceutically acceptable” means approved by a regulatory agency of a Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

A pharmaceutical composition sometimes is provided as a pharmaceutical pack or kit comprising one or more containers filled with a therapeutic composition of cells prepared by a method described herein, alone or with such pharmaceutically acceptable carrier. Additionally, one or more other prophylactic or therapeutic agents useful for the treatment of a disease can also be included in the pharmaceutical pack or kit. A pharmaceutical pack or kit may include one or more containers filled with one or more of the ingredients of the pharmaceutical compositions described herein. Optionally associated with such container(s) can be a notice in the form prescribed by a governmental agency regulating the manufacture, use or sale of pharmaceuticals or biological products, which notice reflects approval by the agency of manufacture, use or sale for human administration. A pharmaceutical pack or kit sometimes includes one or more other prophylactic and/or therapeutic agents useful for the treatment of a disease, in one or more containers.

When the cells are expanded ex vivo before being administered as an immunotherapy, the cells sometimes do not make a sufficient amount of cytokines on their own. Cytokines that can optionally be added to the kits provided herein include, but are not limited to, TNF, IFNγ, interleukins IL-1s, IL-4, IL-6, IL-7, IL-10, IL-12, IL-18, IL-21, CCL4/RANTES, and TGFβ. In certain aspects, the pharmaceutical compositions and/or kits provided herein optionally include a second agent for co-administration with the compositions provided herein. The words “co-administer,” “co-administers,” co-administration” and the like, as used herein, means that the agent is administered before, after or concurrently with the innate cell compositions provided herein.

In some aspects, the second agent is an antibody that targets a cancer cell antigen or an infectious pathogen. The compositions, pharmaceutical compositions and kits provided herein can be maintained at negative 4 degrees Celsius or less, or at about negative 75 degrees Celsius to about negative 80 degrees Celsius, as appropriate for storage or transportation.

### Methods of Making Genetically Modified Innate Cell Compositions

The innate cell compositions provided herein can be genetically modified in a suitable manner (e.g., by adding an exogenous polynucleotide that is a gene or a regulatory sequence, by mutating an endogenous gene, or by deleting an endogenous gene). The genetic modification can be performed after the final composition is obtained, following activation and expansion, or it can be performed following activation and before the cells undergo expansion. Non-limiting examples of methods of making various genetic modifications are described hereafter:

(a) CRISPR-CAS9 Targeted Suppression (Permanent Gene/Locus Deletion)
Cells can be transfected with a DNA plasmid that expresses both the CAS9 protein and a guide RNA (gRNA) specific for the gene of interest. The gRNA-CAS9-mediated cut in the genome can be repaired using a donor DNA plasmid, which causes specific deletion of the targeted gene and permanent and total loss of the gene-encoded protein. Loss of protein expression can be validated using PCR (DNA level), Northern Blot/FISH (RNA level), or any Protein assay such as, for example Western blot or flow cytometry.

(b) CRISPR-CAS9 Targeted Expression (Permanent Gene/Locus Insertion)
This method can be used to insert the gene of interest into a specific location of the cell genome. Cells can be transfected with a DNA plasmid that expresses both the CAS9 protein and a guide RNA (gRNA) specific for the specific insertion location. The gRNA-CAS9-mediated cut in the genome can be repaired using a donor DNA plasmid, which has the inserted gene of interest flanked by sequences of the cell genome on both sides of the location of the DNA cut/double stranded break, causing homologous recombination-mediated insertion of the gene of interest in the specific genome location rather than randomly. Successful insertion and protein expression can be validated using PCR (DNA level), Northern Blot/FISH (RNA level), or any suitable protein assay such as, for example, Western blot or flow cytometry.

(c) RNA Interference by Retroviral/Lentiviral/Transposon-Mediated Transduction of shRNA/microRNA (Permanent Gene Suppression)
shRNA/microRNA targeting the specific gene/protein of interest can be designed and cloned into a retroviral/lentiviral/transposon vector for stable integration into the cell genome. Cells can be transduced with the vector and successfully transduced cells can be selected using the vector encoded selection markers. shRNA-mediated suppression of the gene of interest can be evaluated using, e.g., Northern Blot and protein assays.

(d) Lentivirus/γ-Retrovirus-Mediated Random/Multiple Copy Gene Insertion
The specific gene/protein of interest can be designed and/or cloned into a retroviral or lentiviral vector for stable random integration into the cell genome. Cells can be transduced with the viral vector and successfully transduced cells can be selected using the vector encoded selection markers. shRNA-mediated suppression of the gene of interested can be evaluated using Northern Blot and any suitable protein assays, such as western blot, flow cytometry, and the like.

(e) Transposon-Mediated Random/Multiple Copy Gene Insertion
The specific gene/protein of interest can be designed and/or cloned into a mammalian transposon vector system such as the PiggyBac (SBI System Biosciences) or equivalent. Cells can be co-transfected with the transposon vector with the gene (cDNA) of interest flanked by the inverted terminal repeat (ITR) sequences and the Transposase vector. The Transposase enzyme can mediate transfer of a gene of interest into TTAA chromosomal integration sites. Successfully transduced cells optionally can be selected using vector encoded selection markers. Successful insertion and protein expression can be validated using PCR (DNA level), Northern Blot/FISH (RNA level), or any suitable protein assay such as, for example Western blot or flow cytometry.

(f) Direct Transfection
mRNA encoding the gene/protein of interest can be transfected directly into the cells. Transfection can be performed using any of the established methodologies, e.g.: calcium chloride transfection; lipofection; Xfect; electroporation; sonoporation and cell squeezing (e.g., to introduce siRNA).

### Methods of Treatment

Also provided herein are methods for treating a cancer or an infection by administering to a subject in need thereof any of the compositions, pharmaceutical compositions or kits provided herein in an amount effective to treat the cancer or infection. The treatment can be administered in an autologous setting or in an allogeneic sitting. The donor of the sample from which the composition, pharmaceutical composition or kit is produced can be the recipient of the treatment. Often, the composition, pharmaceutical composition or kit is prepared from a sample from one subject, and the treatment is administered to a different subject. In certain aspects, the treatment can be administered on two or more separate days and in certain aspects, the treatment can be administered in multiple doses. In certain aspects, the treatment is administered at between about 1 unit dosage to about 36 or more unit dosages at intervals of between about 2 weeks to about 4 weeks. In some aspects, the treatment is administered as a single unit dosage one two, three, four or up to five times daily, or one, two, three, four, five, six, seven, eight, nine or ten or more times over the course of several days, weeks or months, or every other day, or one, two, three four, five or six times a week. The treatment can be administered intravenously (IV), intrathecally or intramuscularly (IM), intraperitoneally (IP), intra-pleurally, into the joint space or is injected or implanted at or near the site of the cancer or infection, and at a unit dosage of between about 104 to about 10¹⁰ cells per kilogram of weight of the subject, or between about 10⁶ to about 10¹² cells per subject. In certain aspects, the unit dosage is about 10¹⁰ cells per subject, or about 10⁸ cells per kilogram of weight of the subject.

In certain aspects of the methods of treatment provided herein, the treatment is for cancer. In some aspects, the cancer is a solid tumor. In aspects, the cancer is a hematological cancer. In certain aspects, the cancer is a hematological cancer and the ratio of gamma.delta T cells to NK cells is greater than one. In some aspects, the cancer is a solid tumor and the ratio of NK cells to gamma.delta T cells is greater than 1. In certain aspects of the methods of treatment provided herein, a second agent is co-administered with the composition, pharmaceutical composition or kit. In some aspects, the second agent is an antibody that immunospecifically binds to a cancer-associated antigen.

In certain aspects of the methods of treatment provided herein, the treatment is for an infection. In some aspects, the infection is characterized by the presence of a bacterial, fungal, viral or protozoan pathogen.

In certain aspects of the methods provided herein, the compositions, pharmaceutical compositions or kits contain a polyconal population of gamma.delta T cells (e.g., polyclonal with respect to V.delta.1 and V.delta.2 gamma.delta T cells).

The technology will further be described with reference to the examples described herein; however, it is to be understood that the technology is not limited to such examples.

### EXAMPLES

The examples set forth below illustrate certain embodiments and do not limit the technology.

The following materials were used in certain of the methods (e.g., activation conditions, expansion conditions, phenotypic analyses) described below:

The following monoclonal antibodies (mAbs) were generated at the Ospedale Pediatrico Bambino Gesù (OPBG), “Cell and Gene Therapy for Pediatric Tumor” Laboratory:

c218 (IgG1, anti-CD56), c127 (IgG1, anti-CD16), AZ20 and F252 (IgG1 and IgM, respectively, anti-NKp30), BAB281 and KL247 (IgG1 and IgM, respectively, anti-NKp46), Z231 (IgG1, anti-NKp44), ECM217 and BAT221 (IgG2b and IgG1, respectively, anti-NKG2D), KRA236, GN18 and F5 (IgG1, IgG3 and IgM, respectively, anti-DNAM-1), EA4 (IgG2a, anti-CD18), MAR206, MA258 and QA196 (IgG1, IgG2b and IgM, respectively, anti-CD2), MA127 and ON56 (IgG1 and IgG2b, respectively, anti-NTB-A), PP35, ST39 and C054 (IgG1, IgG1a and IgM, respectively, anti-2B4), z27 (IgG1, anti-KIR3DL1/S1), AZ158 (IgG2a, anti-KIR3DL1/L2/S1), z270, z199 and Y9 (IgG1, IgG2b and IgM, respectively, anti-NKG2A), 6A4 and A6/136 (IgG1 and IgM, respectively, anti-HLA-class 1), D1/12 (IgG2a, anti-HLA-DR), 5A10 (IgG1, anti-PVR), L14 (IgG2a, anti-Nectin-2), BAM195 (IgG1, anti-MICA).

The F278 (IgG1, anti-CD85j) mAb was kindly provided by Dr. Daniela Pende, Istituto Giannina Gaslini, Genoa, Italy (see also, e.g., Costa et al., Aids, 15:965-974 (2001)). Anti-NKG2C (IgG2b, 134522 clone), anti-ULBP-1 (IgG2a, 170818 clone), anti-ULBP-2 (IgG2a, 165903 clone), anti-ULBP-3 (IgG2a, 166510 clone), anti-CD34-APC (IgG1, QBEnd10 clone), IgG-APC Isotype Control (IgG1 clone 11711) and anti-KIR2DL1-FITC or non-conjugated (IgG1, 143211 clone) mAbs were purchased from R&D System Inc (Abingdon, United Kingdom). Anti-KIR2DL1/S1-Vioblue or -PE (IgG1, 11PB6 clone), anti-NKG2C-ViobrightFITC (REA205 clone), anti-KIR3DL1-biotin or -FITC (IgG1, DX9 clone), anti-CD3-Viogreen (IgG2a, BW264/56 clone), anti-CD57-Vioblue (IgM, TB03 clone), anti-SIGLEC-7-Vioblue (REA214 clone), anti-NKp30-PE (IgG1, AF29-4D12 clone), anti-NKp46-PE (IgG1, 9E2 clone), anti-NKp44-PE (IgG1, 2.29 clone), anti-biotin-PerCPVio700 (REA746 clone), REA control VioBright FITC (clone REA293), anti-PD-1 (IgG2b, PD1.3.1.3 clone), mAbs were purchased from Miltenyi Biotec (Bergisch Gladbach, Germany). Anti-CD34 (IgG1, QBEnd10 clone), anti-NKG2A-PC7 (z199 clone), anti-KIR3DL1/S1-PE (z27 clone), anti-KIR2DL2/L3/S2-PE (GL183 clone), anti-CD19-FITC (IgG1, J3-119 clone), anti-CD56-PC7 (IgG1, N901 clone), IgG1-PC7 or -PE or -FITC Isotype Control (679.1Mc7 clone) mAbs were purchased from Beckman Coulter, Immunotech (Marseille, France). Anti-KIR2DL2/L3-S2-FITC or non-conjugated (IgG2b, CHL-clone), anti-CD107-PE (IgG1, H4A3 done), anti-CD85j (IgG2b, GHI/75 done), anti-CD16-PerCpCy5.5 (IgG1, 3G8 done), anti-CD56-BV510 (IgG2b, NCAM16.2 done), IgG1-PE Isotype Control (done MOPC-21) mAb and Brillant Stain Buffer was obtained from BD Bioscience Pharmingen (San Diego, Calif.). Anti-HLA-Bw6-FITC and anti-HLA-Bw4-FITC mAbs were purchased from ONE LAMBDA INC (Canoga Park, CA). Anti-human HLA-E (IgG1, 3D12 clone) and anti-human HLA-G (IgG1, MEM-G/9 done) mAbs were purchased from BioLegend (San Diego, Calif.) and Abnova (Taipei, Taiwan) respectively. Anti-NKG2D (IgG2a, 5C6 clone) and anti-HLA-C (IgG1, C-8 done) were purchased from Santa Cruz Biotechnology (Dallas, Tex., USA).

For the expansion conditions, IL-2, IL-15 and OKT3 were obtained from Miltenyi Biotec (San Diego, Calif., USA), as were anti-CD2 and anti-NKp46 antibodies in crosslinked for or bound to beads.

### Example 1: Enrichment and Expansion of Specific Population of Innate Cytolytic Immune Cells (INNATE-19 from the αβTCRneg Cell Population

This Example describes a process that enriches and expands a specific innate cytolytic immune cell population (referred to herein as an INNATE-K or a BINATE cell population) consisting largely of NK and γδTCR+ T cells from a starting αβTCRneg cell population (see FIG. 2 for an analysis of the composition of this expanded T cell population; the terms INNATE-K and BINATE may be used interchangeably herein, and refer to cell populations discussed herein that are activated with a combination of an NCR antibody (e.g., anti-NKp46) and an LFA antibody (e.g., anti-CD2) and contain a mixture of NK cells and γδT cells), in feeder-free culture conditions, activated with antibodies to one or more of the immunoglobulin superfamily surface molecules:

1. A large scale leukapheresis was performed on a normal healthy donor who had been mobilized with G-CSF; alternatively, a large scale leukapheresis is performed on a normal healthy donor without mobilization, or a buffy coat is used.

2. A gradient cell separation with Ficoll™ was performed with a SEPAX™ device (Sepax Technologies, Inc., Newark Del.), to remove red blood cells, platelets and granulocytes leaving a mononuclear cell suspension; alternative methods to perform Ficoll™ gradient separation include the MILTENYI PRODIGY™ (Miltenyi Biotec, San Diego, Calif.) or manual separation with a centrifuge.

3. The mononuclear cells were subjected to αβ T cell depletion using clinical scale MILTENYI CLINIMACS™ (Miltenyi Biotec, San Diego, Calif.) per the manufacturer's instructions, or were subjected to the research scale Miltenyi LS-column separation.

4. The resulting αβTCRneg cell population was placed in feeder-free culture conditions and activated by anti-CD2 and anti-NKp46 beads (NK Cell Activation/Expansion Kit™ (Miltenyi Biotec, Inc., San Diego, Calif., USA)), and 500 IU/mL human Interleukin 2 (IL-2) (Miltenyi Biotec) following the manufacturer's instructions.

5. The activated αβTCRneg cell population was further culture-expanded in feeder-free culture conditions with NK MACS™ Medium (#130-107-879 (Miltenyi Biotec, Inc., San Diego, Calif., USA)) supplemented with 5% AB serum and 500 IU/mL human Interleukin 2 (IL-2), at the cell seeding concentration of 0.25×101/ml in a 24 well plate then transferred to a T75 flask; alternative culture vessels could include a bioreactor (G-Rex™ 25 ml, Wilson Wolf Manufacturing, St. Paul Minn.).

6. Media exchanges took place approximately every 3 days with fresh medium and fresh IL-2, in concentrations described in #5 and cells were seeded at a concentration of 0.25-0.5×10⁶/ml. In certain variations of the method, media exchanges take place approximately every 4 days. When seeded in bioreactors, the media exchanges may occur approximately every 6 or 7 days until target dose is achieved.

7. At any time after initiation of expansion, the resulting enriched specific innate immune cytolytic population (NK and γδTCR+ T cells) could be enriched further for either pure NK or γδTCR+ T cells to obtain pure cell populations using CD3+ depletion or CD56+ depletion, respectively, of the undesired population and further cultured as in step #5.

8. At the end of expansion INNATE-K cells are cryopreserved in serum-free freezing media containing 10% DMSO solution (CryoStor™, BioLife Solutions, Bothell Wash.).

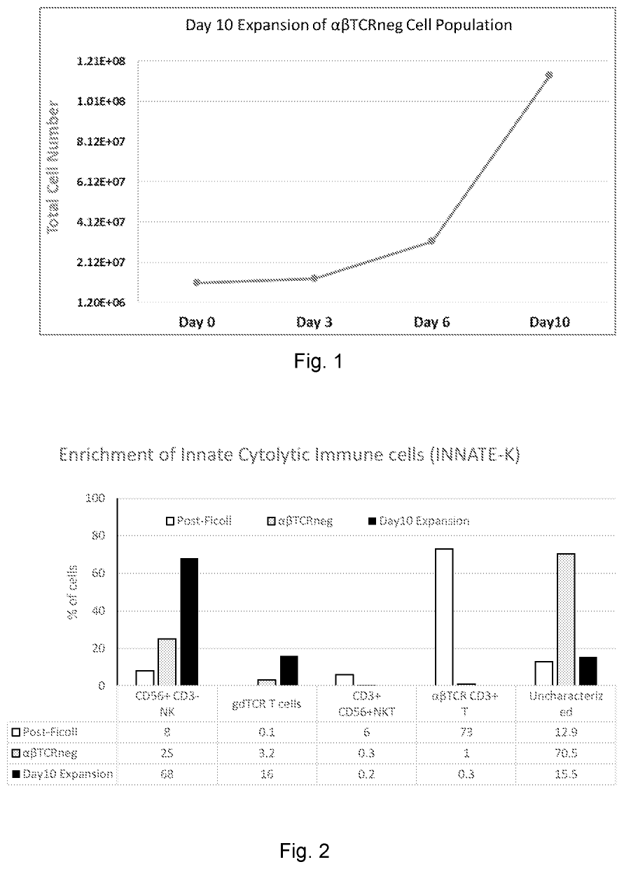

FIG. 1 illustrates the expansion of the αβTCRneg cells as a function of days the cells were activated with antibodies and expanded in the feeder-free culture conditions as analyzed on day 10 of culture. The graph shows that the cells began to attain logarithmic expansion around 1 week and continued to expand, yielding large numbers suitable for a successful off the shelf therapy. In FIG. 1, activated αβTCRneg cells were culture-expanded in feeder-free culture conditions. The total cell number of the culture by day of expansion is shown, with significant increase in expansion seen after day 6 in culture.

FIG. 2 shows NK cells at 70% and γδTCR+ T (GD) cells at 28%; this proportion is suitable for a therapeutic product; there can be shifts as the cells are expanded further; these two cell types were obtained at high numbers without feeder cell culture and without contamination from other cells. FIG. 2 illustrates the enrichment and phenotype of the specific innate immune cytolytic cells (INNATE-K), NK and γδTCR+ T cells, as well as CD3+CD56+ NKT cells and adaptive immune αβTCR+ T cells at the various steps in the following example of a manufacturing process:

a) frequency of cells in the mononuclear fraction after Ficoll gradient separation was 8%+12% NK cells and 0.1%+1% γδTCR+ T cells, 6% CD3+CD56+ NKT cells and 73% αβTCR+ T cells;

b) frequency of cells after the αβTCR+ T cell depletion step was 25%+15% NK cells and 3.2%+1.2% γδTCR+ T cells (of which 0.5% CD56− CD3+ and 2.7% CD56+CD3+) cells and 1% or less αβTCR+ T cells.

c) on day 10 after activation and feeder free culture expansion the NK cell frequency was substantially enriched to 68%+23% and γδTCR+ T cells substantially enriched to 15.7%+5% (of which 12.2% CD56− CD3+ and 3.5% CD56+CD3+). Negligible levels of CD3+ adaptive immune cells were detected: αβTCR+ T cells at 0.3%+0.5% and NKT cells detected at 0.2%+0.3%.

A day 10 flow cytometry analysis (fluorescent activated cell sorter, or FACS analysis) of cells co-expressing the CD56 (NK) surface antigens, CD16, and CD57 was performed. CD16 is the FCγ receptor III, and can thus bind the FC portion of IgG antibodies and mediate antibody dependent cell-mediated cytotoxicity (ADCC) of antibody-bound target cells. CD16 has a role in NK cell-mediated spontaneous cytotoxicity. The CD56+CD16+ subset is considered the most cytotoxic subset and makes up the majority of NK cells (e.g., under physiological conditions). CD57 may be a marker of NK cells with poor proliferative capacity. Acquisition of CD57 on NK cells—following stimulation with IL-2 or coculture with target cells—correlates with maturation of the CD56+NK cell subset. This differentiation is accompanied by functional changes; compared with CD57− cells, CD57+NK cells proliferate less well in response to IL-2 and IL-15 and produce less IFN-γ in response to IL-12 and IL-18. For the CD56+NK cells, 37% were CD16+ and 63% were CD16−; for the CD56+CD16− cells, 13% were CD57+ and 87% were CD57−; and for the CD56+CD16+ cells, 15% were CD57+ and 85% were CD57−. The flow cytometry phenotype results indicated that the cellular composition contained enriched NK cells in the expanded αβTCRneg cells that were highly cytotoxic NK cells as evidenced by the co-expression of CD56 (NK), and CD16, yet also contained cells with an immature phenotype with the potential to mature into even more responsive and cytotoxic cells in vivo as evidenced by the numbers of NK cells with no expression of CD57 on both CD16+ and CD16− NK cells.

The distribution of the frequency of the different γδ T cell subsets of the day 10 sample of the feeder-free, culture expanded activated αβTCRneg cells as analyzed by flow cytometry was measured. γδ T cells, in contrast to MHC-restricted αβ T-cells, are capable of recognizing and lysing diverse cancers in an MHC-unrestricted manner, highlighting their potential for off the shelf allogeneic immunotherapy. Human γδ T cells can be divided into three main populations based on δ chain expression. The majority, up to 50%-90%, of circulating γδ T lymphocytes in healthy human adults express the γδ2 chain. γδ T cells expressing γδ1 chains are most prominent in the intraepithelial layer of mucosal surfaces, where they are involved in maintaining epithelial tissue integrity when facing damage, infection, or transformation, however, they may also appear in the peripheral blood. γδ3 T cells make up about 0.2% of circulating T cells including CD4+, CD8+, and CD4-CD8− subsets. While infrequent in the peripheral blood, γδ3 T cells are usually more highly represented in the liver. The percentage distribution of the γδ TCR+ T cells on day 10 (41% γδ1, 50% γδ2, 7% other γδ) clearly shows that propagated γδ T cells were polyclonal as they expressed Vδ1, Vδ2, and Vδ1^{neg}Vδ2^{neg} subsets. This polyclonality will ensure the participation of the propagated γδ T cells in innate killing of tumors, altered cells or infection.

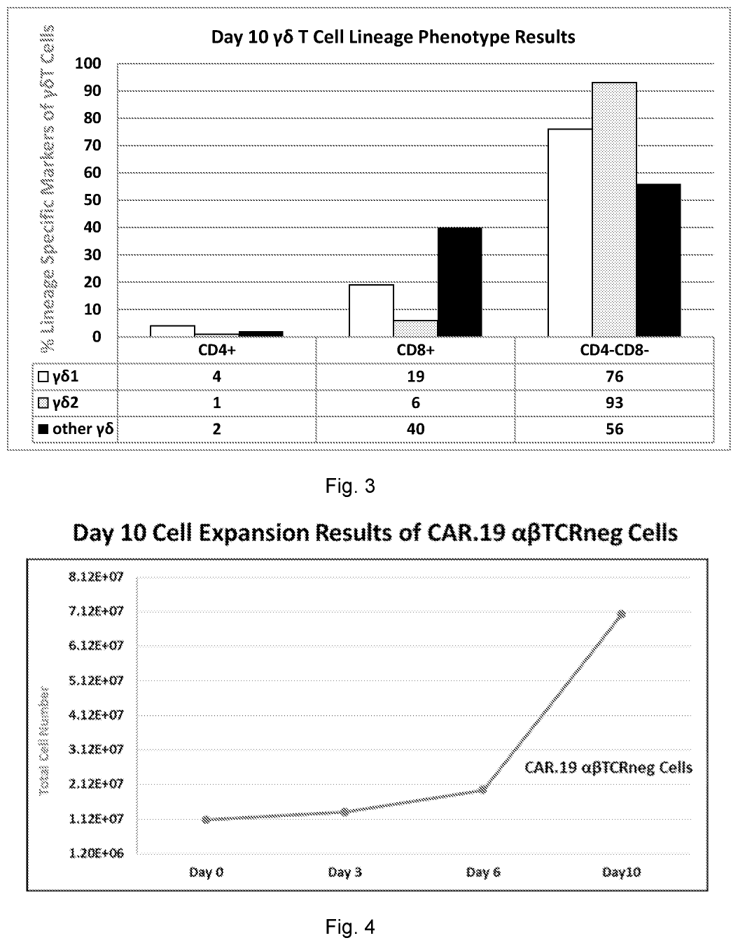

FIG. 3 illustrates the frequency and distribution of the T cell lineage markers, CD4 (helper) and CD8 (cytotoxic) on the γδ TCR+ cells in a day 10 sample of the feeder-free, culture expanded activated αβTCRneg cells as analyzed by flow cytometry. Most of the γδ1 T cells did not express CD4 or CD8, however a small percentage (19%) did express cytotoxic CD8 lineage markers, which is consistent with the innate cytolytic immune population that is described and desired for maximum tumor and infection killing. γδ2+ cells expressed little of either CD4 or CD8, while almost half of the “other γδ TCR+ cells”, composed primarily of the γδ3 T cells, expressed the cytotoxic CD8 lineage marker.

Additional day 8 flow cytometric phenotypic analytic data were obtained where the γδ T cells were obtained from the feeder-free, culture-expanded activated αβTCRneg cells which had been depleted with the research scale Miltenyi Biotec, Inc. αβT cell depletion reagents. As with the large scale depleted cells, the day 8 culture CD3+ T cells primarily expressed the γδ T cell receptor (<0.05% αβTCR+ cells). A phenotypic analysis was performed and co-expression of markers on the γδ T cells was analyzed. All cells were CD3+ and 31.8% were CD16+. Co-expression of CD57 and CD16 on the CD3+γδ T cells was observed. 10.7% co-expressed the CD57 and CD16 markers and were thus the most mature cells. The majority were CD57− and CD16− (57%), thus representing the immature cells. Expression of the inhibitory and activating ligand receptors was analyzed. NKG2D is an activating receptor on the NK cell surface. NKG2A dimerizes with CD94 to make an inhibitory receptor. The cells lacked expression of NKG2C and 55% of the cells co-expressed NKG2A and NKG2D. A phenotypic analysis was performed and co-expression of markers on the γδ T cells was analyzed in relation to the co-expression of CD56. Of all of the γδ cells which expressed CD3+ and CD56+, 29% co-expressed CD16. Co-expression of CD57 and CD16 was observed on the CD3+CD56+γδ T cells. 5% co-expressed the CD57 and CD16 markers and were thus the most mature cells. The majority were CD57− and CD16− (67%), thus representing the immature cells. Expression of inhibitory and activating ligand receptors was analyzed. Cells lacked the expression of NKG2C on the CD3+CD56+ cells, with the majority of cells (82%) expressing NKG2A, and 80% of the CD3+CD56+ cells co-expressed NKG2A and NKG2D, indicating that these cells expressed the phenotypic markers most commonly associated with NK cells, and which were indicative of an active innate immune cell population.

### Example 2: Expansion of Transduced Specific Innate Cytolytic Immune Cells (INNATE-CAR) from the αβTCRneg Cell Population

This Example describes the expansion and production of a population of transduced specific innate cytolytic immune cells (INNATE-CAR) comprising NK and γδTCR+ T cells, which were expanded from an activated culture of αβTCRneg cell population, in feeder-free culture conditions:

1. An aliquot of cells from the activated, culture expanded αβTCRneg cell population as described in Example 1 step #5 was taken on day 4 (alternatively an aliquot can be taken from day 5-15) and seeded onto a human fibronectin-coated plate at the concentration of 0.25×10⁶/ml in the presence of 500 IU/mL human Interleukin 2 (IL-2) or 10 ng/ml IL-15 (alternatively a RetroNectin™ (Takara Bio USA, Mountain View Calif.) coated plate or VECTOFUSIN-1 (Miltenyi Biotec) transduction enhancer could be used) and transduced with gamma retroviral supernatant containing the construct for the CD+19 chimeric antigen receptor containing a nonfunctional fragment of the CD34 surface antigen which is used as a marker for cell transduction.

2. Three days later, cells were detached and further expanded in NK MACS™ Medium (#130-107-879 (Miltenyi Biotec, Inc., San Diego, Calif., USA)) supplemented with 5% AB serum and 500 IU/mL human Interleukin 2 (IL-2), at the cell seeding concentration of 0.25×10⁶/ml in a 24 well plate or T75; alternative culture vessels could include a bioreactor (G-Rex™ 25 ml, Wilson Wolf Manufacturing, St. Paul Minn.).

3. Media exchanges took place every 3 days with fresh medium and fresh IL-2, in concentrations described in #6 of Example 1, and cells were seeded at a concentration of 0.25-0.5×10/mL; when seeded in bioreactors the media exchanges would occur every 7 days until target dose had been achieved.

4. Anytime after day 14, INNATE-CAR population could be enriched further for either NK-CAR or γδ-CAR T cells to obtain pure cell populations using CD3+ depletion or CD56+ depletion, respectively, of the undesired population and further cultured as in step #3.

5. At the end of expansion cells are cryopreserved in serum-free freezing media containing 10% DMSO solution (CryoStor, BioLife Solutions, Bothell, Wash.).

FIG. 4 illustrates the analysis of expansion rate of CAR.19 INNATE-K cells by 10 days. Similar to the expanded non-gene modified population, the culture demonstrated logarithmic expansion beginning around one week, representing a robust expansion potential for off the shelf INNATE-CAR products. In FIG. 4, activated INNATE-K cells were culture-expanded in feeder-free culture conditions and transduced on day 4 with gamma retrovirus containing a CD19 CAR and a nonfunctional CD34 marker.

Transduction of INNATE-CAR populations, NK cells and γδTCR+ T cells was compared (in three different productions). Specifically, the % CAR.CD19 expression was compared in each of these cell populations using the CD34 marker incorporated in the CAR molecule. Both NK and γδ TCR+ T populations showed significant transduction by the CAR.CD19 retroviral vector as evidenced by analysis of the CD34 marker in the declared cell subsets. Transduction efficiency was on average 33% and 30% for NK and γδ TCR+ T cells, respectively. Flow cytometry analysis (fluorescent activated cell sorter, or FACS analysis) was performed on feeder-free NK cells expanded for 10 days and not transduced with retroviral vector carrying CAR.CD19, which showed the specificity of the CD34+ staining on CD56+ cells. Flow cytometry (FACS) analysis was performed on feeder-free NK cells genetically modified at day +4 from activation process with retroviral vector carrying CAR.CD19 and expanded for 10 days. The results showed a significant level of CAR expression after transduction on CD56+ cells in terms of both the percentage of CD34+ cells and the mean fluorescence intensity of the expression. Both features are associated with highly efficient tumor recognition. The same observation was also valid in the context of feeder-free γδTCR T cells present in the transduced and expanded INNATE population. Indeed, the γδTCR T cell component was significantly transduced, as assessed by the analysis of CD34 expression in γδTCR T cells.

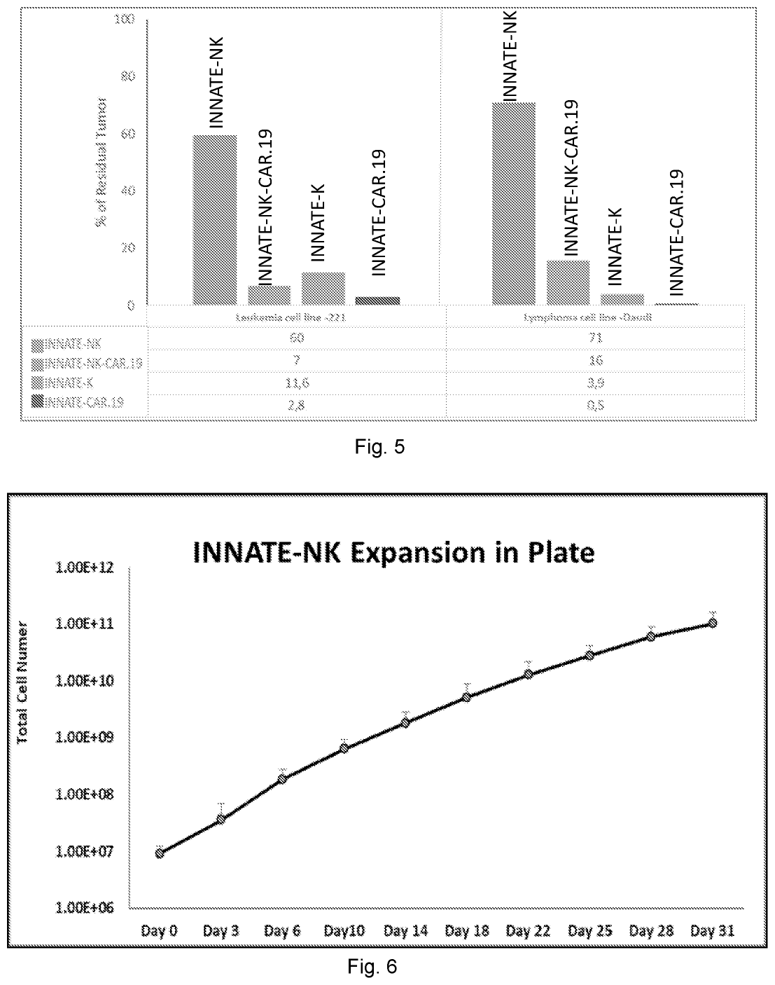

The INNATE-CAR platform is highly efficient in recognizing and eliminating tumor, with a strong synergism between innate activity and the CAR mediated functionality. Indeed, FIG. 5 graphically illustrates data from a co-culture cytotoxicity assay, where INNATE cells and INNATE-CAR.CD19 cells were co-cultured with CD19+ leukemia (221) or CD19+ lymphoma (Daudi) cell lines. The % residual tumor after the culture is represented in the graph above for the different cell effectors. Non-transduced NK cells (INNATE-NK) killed 40 and 29% of leukemia and lymphoma tumor cells, respectively, while the INNATE-K cells characterized by the NK and γδTCR T cell combination population killed 88.4% and 96.1% of the tumor cells, respectively. Transduction of the INNATE-NK cells with the CAR.CD19 (INNATE-NK-CAR.CD19) allowed for 93% and 84% elimination of the tumor cells, while the INNATE-CAR.CD19 combination population killed almost all tumor cells (97% and 99%).

### Example 3: Alternative Enrichment of INNATE-NK Cells

This Example describes a process for the enrichment of INNATE-NK cells:

1. NK cells were isolated from buffy-coat with NK DEPLETION KIT™ (Miltenyi Biotec, Inc., San Diego, Calif., USA).

2. NK cells were activated with NK Cell ACTIVATION/EXPANSION KIT™ (Miltenyi Biotec, Inc., San Diego, Calif., USA) and recombinant human IL-2 (500 U/ml).

3. Day 0, post activation, NK cells were cultured in 5% AB serum-enriched media (NK MACS™ Medium (#130-107-879 Miltenyi Biotec, Inc., San Diego, Calif., USA).

4. Day 4 to 15, an aliquot of cells was subjected to transduction in human fibronectin-coated plate at the concentration of 0.25×10⁶/ml in the presence of 500 IU/mL human Interleukin 2 (IL-2).

5. Three days later cells were detached and further expanded in 5% AB serum-enriched media and 500 IU/mL human Interleukin 2 (IL-2), at the cell seeding concentration of 0.25×10⁶/ml.

6. After transduction NK cells were plated in 24 well plate then T-flask or Bioreactor.

7. A media exchange took place every 3 days with fresh medium and fresh IL-2, and cells seeded at a concentration of 0.25-0.5×10°/mL (when they were in plates) until the target cell number had been reached. Bioreactor cultures were changed every seven days.

8. On day +14, non-transduced and CAR-transduced INNATE-NK cells were re-depleted for CD3+ cells in case of more than 5% of T cell contamination to obtain pure NK cells, and further cultured as step #7.

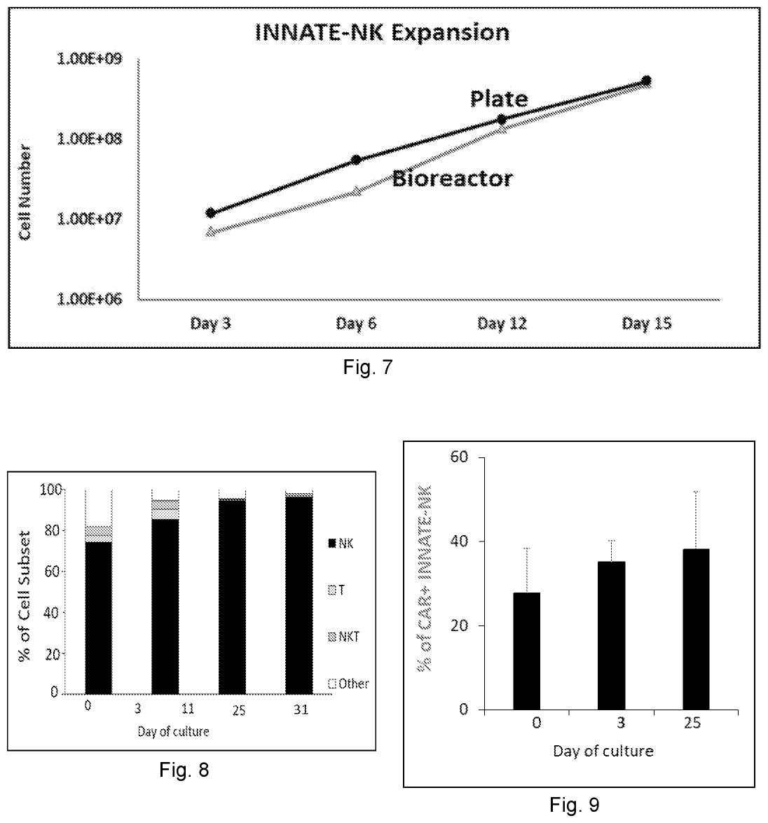

Exemplary highly enriched NK populations greater than 90% (>90%) of innate immune cells expanded robustly in both plates and bioreactors, as graphically illustrated in FIGS. 6-8. FIG. 6 shows total numbers of the expanded INNATE-NK cell population in plates over several days. FIG. 7 compares total cell numbers of plate versus bioreactor expanded INNATE-NK populations. FIG. 8 shows a subset cell composition during the different time points of the in vitro expansion, showing the significant purity level of INNATE-NK, represented by more than 95% of NK cells, and a negligible presence of CD3+ T cells, and NKT cells.

Levels of CAR transduction efficiency was demonstrated using flow cytometry (FACS) analysis evaluating the CD34 marker included in CAR.CD19 molecule. Feeder-free expanded non-transduced INNATE-NK cells do not express CD34 marker. INNATE-NK cells genetically modified at day 4 with retroviral vector carrying CAR.CD19. INNATE-NK CAR cells were significantly transduced with a high percentage of CAR+ cells in the NK population (CD56+). CAR molecule expression in INNATE-NK CAR cells was stable during prolonged in vitro culture, as shown in FIG. 9.

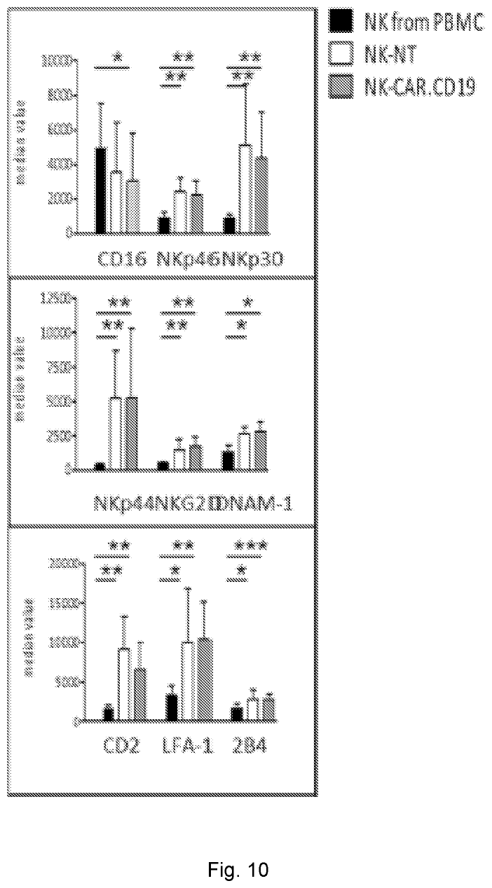

Increased expression of activation and cytolytic molecules after expansion of INNATE-NK and INNATE-NK CAR population is illustrated in FIG. 10, which graphically shows the data from flow cytometric analysis of INNATE-NK and INNATE-NK-CAR.19 cells respectively. The analysis included the evaluation of several NK markers associated with activation and maturation of the innate cells. In particular, CD16 was included as a basic marker for maturation analysis, being associated with a more mature NK subgroup with high cytolytic activity but low proliferative ability; signaling molecules and costimulatory molecules for the activation and proliferation of the NK populations, including CD2, LFA-1, NKp44, NKp30 and NKp46 and DNAM-1. All of these markers were significantly increased in the expanded populations of both the INNATE-NK and the INNATE-NK.CAR.19 cells, indicating that the cells are activated compared to circulating peripheral blood NK cells.

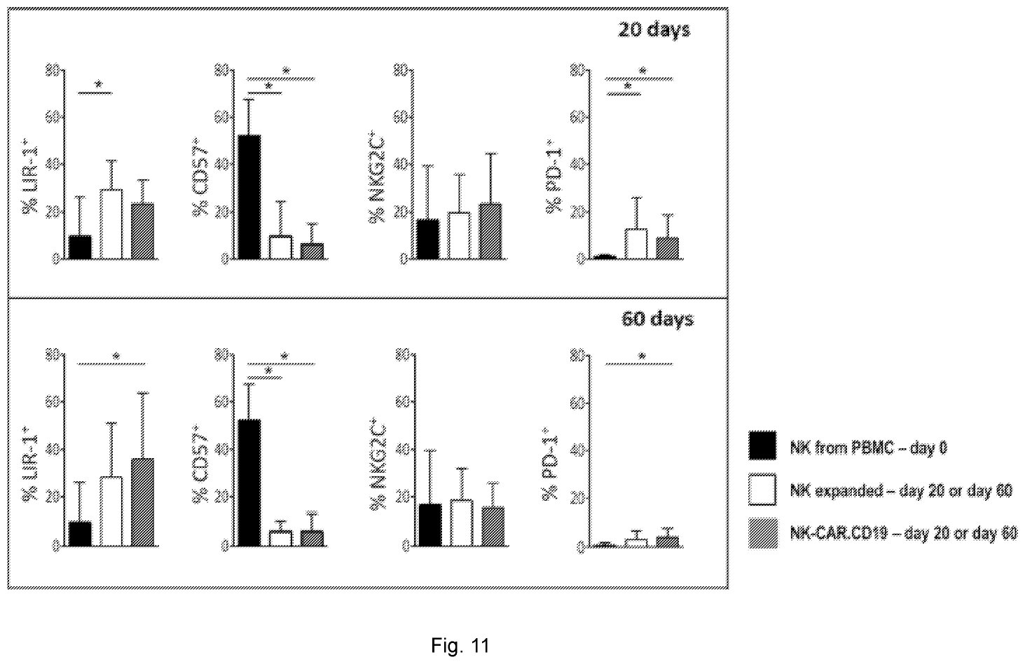

FIG. 11 graphically illustrates the lack of exhaustion in feeder-free expanded INNATE-NK cells and INNATE-NK CAR cells. INNATE-NK and INNATE-NK-CAR.19 cells were generated and expanded as described in Example 3 and flow cytometric analysis was performed on day 20 and day 60 to evaluate markers of cell maturity and exhaustion. LIR-1 is a marker of innate cell inhibitory activity and showed only a minor increase in the NK population at 20 days and in both populations at 60 days; NKG2c is a marker of signal specific activity and maturity and did not change over the period of culture; CD57 is a marker of maturity and it is associated with decreased proliferative potential, and thus it is expressed on the circulating NK population derived from peripheral blood of healthy donors. Feeder-free expanded INNATE-NK cells at 20 days and 60 days of in vitro expansion showed a significant low percentage of CD57+NK cells, suggesting that these cells would still have proliferative potential upon in vivo infusion. Programmed cell death 1 molecule (PD-1) is a marker of decreased cell potential and “exhaustion” not only in T cells but it was recently described as checkpoint inhibitor also in NK cells. Neither population of INNATE-NK and INNATE-NK CAR cells at either 20 days or 60 days showed the presence of PD-1+ cellular subsets, showing that the cells were not exhausted after extended in vitro culture.

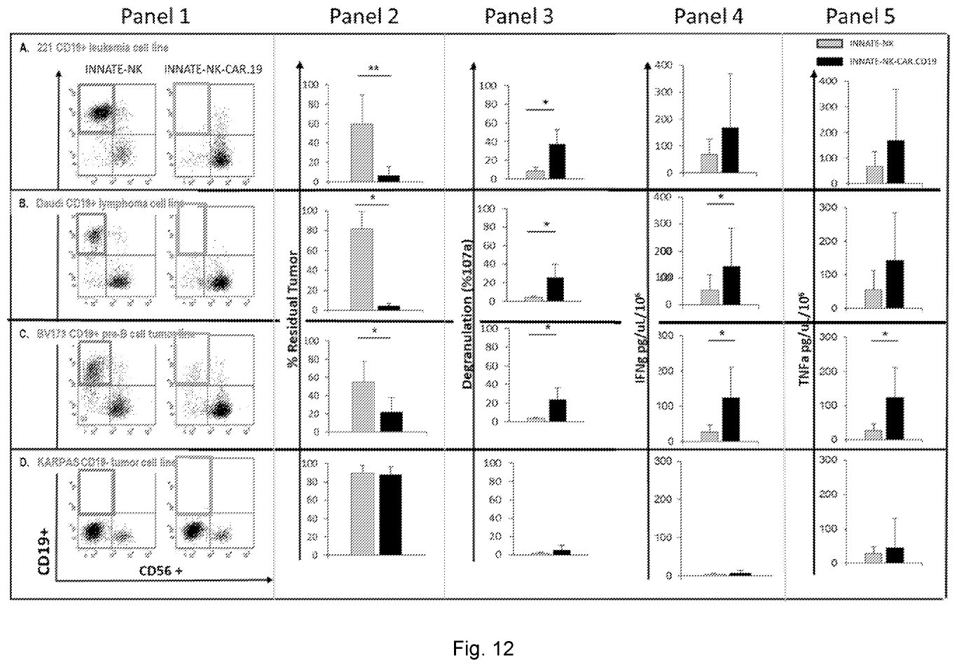

FIG. 12 graphically illustrates a cytotoxic co-culture assay with INNATE-NK or INNATE-NK-CAR.19 and 4 tumor cell lines: Row A: 221, a CD19+ leukemia cell line; Row B: Daudi, a CD19+ lymphoma cell line; Row C: BV173, a CD19+(variable expressing) pre-B tumor cell line; and Row D: KARPAS, a CD19− tumor cell line. FIG. 12 Panel 1 shows the reduction in CD19+ cells after in vitro co-culture with INNATE-NK (left subpanel) or INNATE-NK.CAR.19 (right subpanel) cells. The upper left-hand box in the INNATE-NK panels (outlined box) highlights the CD19+ tumor population after co-culture with the unmodified INNATE-NK cells. The upper left-hand box in the INNATE-NK.CAR.19 panels (outlined box) highlights the residual CD19+ tumor population after co-culture with the INNATE-NK.CAR.19 cells. The INNATE-NK.CAR.19 cells were able to exert significant tumor control against CD19+ 221 and DAUDI tumor targets, with a negligible amount of residual tumor after five days of in vitro coculture. BV173 showed a higher resistance to recognition and elimination, although, the genetic modification of INNATE-NK cells with CAR.CD19 was able to significantly increase the antitumor activity compared to un-modified INNATE-NK cells. The average of residual tumor observed in 10 different experiments is shown for each of the co-culture experiments in FIG. 12, Panel 2. Degranulation (analyzed by the expression of CD107a by NK cells after short in vitro coculture of three hours) is a representation of the cytolytic activity of the INNATE-NK and INNATE-NK CAR effector cells it is shown in FIG. 12, Panel 3 (rows A-D). In all cases the INNATE-NK.CAR.19 cells expressed more cytolytic activity than the INNATE-NK cells; in the negative control represented by KARPAS co-culture condition, neither population expressed the CD107a degranulation marker. FIG. 12, Panels 4 and 5, show the production of regulatory cytokines which are generally produced in the case of lymphocyte cytolytic activity. Both of these cytokines have been very elevated during CD19 CAR T cell administrations and are thought to potentially play a role in the cytokine release syndrome. In the cases of INNATE-NK and INNATE-NK CAR activity towards CD19+ tumor cells, the cytokine production of both IFNα and TNFα ranged from 20-150 pg/ml/10⁶ cells, many fold less than that of adaptive T cell killing. The magnitude of cytokine secretion even with high target killing is not as great as that seen with CAR-T (typically 100 to 1,000 fold higher), therefore representing a potentially less toxic therapy.

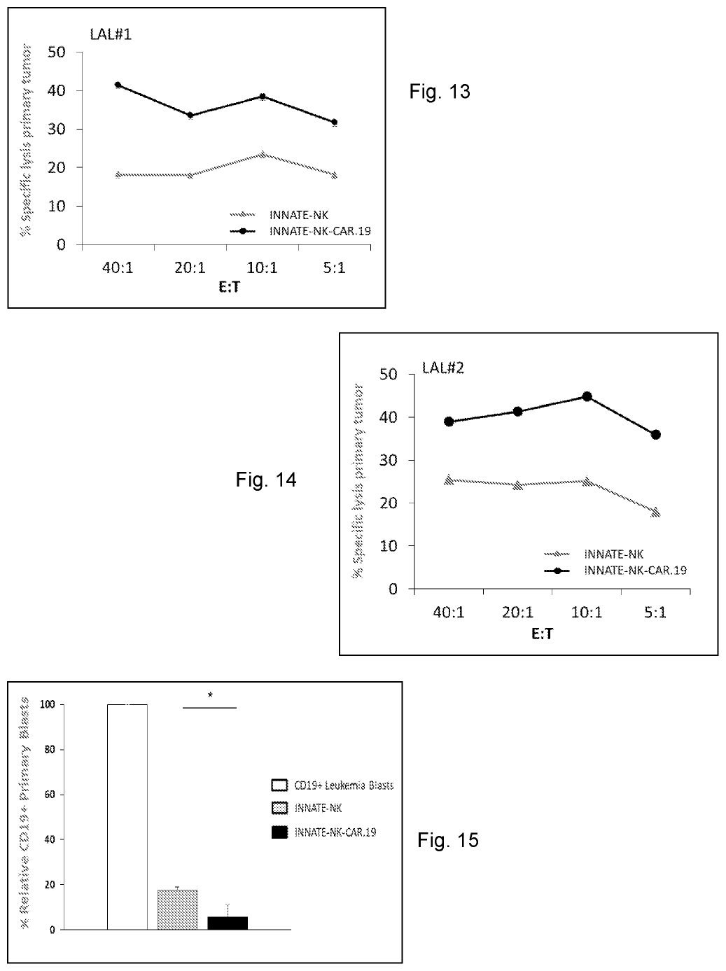

FIGS. 13-15 graphically illustrate that INNATE-NK and INNATE-NK CAR− cells are able to exert a significant antitumor activity towards primary Bcp-ALL blasts. FIGS. 13 and 14 graphically illustrate the % specific lysis of primary tumor cells as a function of the ratio of effector (E) to target (T), for both INNATE-NK cells and INNATE-NK-CAR.19 cell populations in different test runs; the INNATE-NK-CAR.19 cell population was more effective over all of the E:T ranges. FIG. 15 graphically illustrates percentage residual primary CD19+ tumor after the co-culture with the effector cells (INNATE-NK cells and INNATE-NK-CAR.19 cell populations) compared to the control condition in which primary CD19+ leukemia blasts were plated in the absence of effector cells. Co-culture with INNATE-NK cells resulted in approximately 18% residual tumor cells and co-culture with INNATE-NK-CAR.19 cell populations resulted in approximately 5% residual tumor cells.

In one example in vivo CD19+ lymphoma mouse model, each NGS mouse received an IV administration of 0.25×10⁶ tumor cells genetically modified with firefly luciferase so they could be analyzed with biofluorescence analysis. After 3 days, leukemia was shown to be established in the injected mice, allowing their randomization in the treatment cohort of mice receiving an IV infusion of 5×10⁶ INNATE-NK or INNATE-NK-CAR.19. Animals were analyzed for the presence of luciferase+ tumor cells over the course of the experiment. The data were compared to data from another experiment with the same design however in this experiment the animals received 5×10⁶ unmodified adaptive T cells.

### Example 4: INNATE-NK Studies

This Example describes data showing that one dose of INNATE-NK cells without gene modification was able to extend the time to death of the animals compared to the same dose of the adaptive T cells. 0.25×10⁶ tumor cells were administered IV and allowed to expand for 3 days. 5×10⁶ INNATE-NK or INNATE-NK-CAR were administered IV, and the data were compared to data from an experiment with 5 M adaptive T cells. In mice receiving INNATE-NK CAR.19, the tumor control was total, with no tumor biofluorescence detected after day 11 demonstrating that the INNATE-NK-CAR19 eradicated the tumor without any relapse through day 72 (end of experiment). Importantly, the INNATE-NK cells also did not demonstrate xenogeneic responses (seen at this time point historically with CAR T cells). The data also showed that one dose of INNATE-NK cells without gene modification was able to extend the overall survival of the animals compared to the same dose of the adaptive T cells (day 50 versus day 28, respectively). FIG. 16 graphically illustrates the survival curve of the animals from the experiment as described above, receiving INNATE-NK and the INNATE-NK-CAR.19 cells. The data curve of the INNATE-NK.CAR.19 recipients demonstrated improved survival compared to those receiving unmodified INNATE-NK cells.

### Example 5: INNATE-NK Cells Directed Against Solid Tumor

This Example demonstrates that INNATE-NK cells are a suitable platform for treating solid tumors. In this example, the Neuroblastoma model was considered. INNATE-NK and INNATE-NK CAR cells were generated as described in Example 3, steps 1-8. INNATE-NK cells were genetically modified at day 4 with a retroviral vector carrying a third generation CAR specific for GD2 antigen, expressed by neuroblastoma tumor cells. The results demonstrated INNATE-NK CAR.GD2 generation, showing a significant level of CAR expression in CD56+ cells evaluated by flow cytometry (FACS) analysis by staining with the anti-idiotype antibody (1A7). The purity of NK population in INNATE-NK CAR.GD2 production was evaluated in terms of the negligible percentage of CD3+ cells, whereas the level of CAR+ cells was evaluated as 1A7+ cells. The expression of the CAR.GD2 in INNATE-NK CAR was stable during extended in vitro culture (measured at day 8, day 15, day 25, day 31, day 35).

### Example 6: Evaluating the Role of Innate Immunity in Solid Tumor Penetration

This Example evaluated the role of innate immunity in its capacity to penetrate solid tumors. To that effect, 3-D neuroblastoma tumor cell spheroids were grown in MATRIGEL™ culture, and then the adaptive T cells, the INNATE-NK or the INNATE-NK-CAR.GD2 cells were introduced and assessed for their ability to invade the spheroids. Specifically, a solid 3-D tumor model was created with SH-SY5Y neuroblastoma tumor cells which were genetically modified to express green fluorescent protein (GFP) and then cultured in vitro to form solid 3-D tumor spheres. The adaptive peripheral blood T cells, unmodified INNATE-NK cells and INNATE-NK.CAR.GD2 were labeled with a red fluorescence-emitting cell marker and incubated with the tumor spheres. The neurospheres were assayed by 3-D fluorescent microscopy in 3 planes in order to assess the location of the cells in the tumor spheres, and their relative ability to invade the tumor. Confocal microscope images of adaptive T cells (red), showed that the adaptive T cells were not able to actively penetrate the 3-D tumor, as they were located only at the external surface of the tumor spheres (green), as was observed in all 3 planes, and did not migrate into the center of the tumor. Further images showed that the INNATE-NK cells (red) began to breach the surface of the tumor sphere, as observed in all 3 planes; and the INNATE-NK-CAR.GD2 cells breached the surface of the tumor sphere, indicating that these cells have the ability to migrate some distance into the tumor, observed in all 3 planes.

### Example 7: Cross-Linked Vs. Soluble Activation

As described in Example 1, a cell population was generated in culture after activation with a cross-linked natural cytotoxicity receptor (NCR) antibody (such as anti-NKp46) and a lymphocyte function-associated antigen (LFA) antibody (such as anti-CD2) combination (“coated beads”; Miltenyi Biotec). Cell populations discussed herein that are activated with a combination of an NCR antibody (e.g., anti-NKp46) and an LFA antibody (e.g., anti-CD2) and contain a mixture of NK cells and γδT cells are referred to herein as “BINATE” cell populations (and in certain Examples above, are referred to as “INNATE-K” cells). In this Example, a resulting BINATE population was analyzed after activation with either 1) a combination of cross-linked anti-NKp46 (clone 9E2 500 ng/ml; Miltenyi Biotec) and cross-linked anti-CD2 (OKT11 500 ng/ml), or 2) a combination of soluble anti-NKp46 (clone 9E2 500 ng/ml) and soluble anti-CD2 (clone OKT11 500 ng/ml). Cross-linking was achieved by coating flasks with a combination of the antibodies in PBS for 24 hours prior to the start of the experiment, and then decanting the antibody solution and rinsing with PBS at the time of the experiment (referred to herein as coated plates).

1. A buffy coat from a normal healthy donor underwent gradient cell separation with Ficoll™ performed with manual separation in a centrifuge (in certain variations of the method described in this Example and other Examples herein, a normal healthy donor undergoes an apheresis procedure (apheresis; generally with no Ficoll™ gradient cell separation) and a mononuclear cell product is collected per standard procedure, typically on a Terumo Optia machine). Mononuclear cells were subjected to αβ T cell depletion using a research scale Miltenyi LS-column separation (in certain variations of the method described in this Example and other Examples herein, an apheresis product may undergo αβ T cell depletion using a CLINIMACS separation device; further depletion may include CD19 B cell depletion. Aliquots of the αβ T cell depleted mononuclear cells may be cryopreserved and later used for expansion). The resulting αβ TCR-neg cell population was activated in feeder-free culture, with BINATE medium (i.e., NK MACS™ Medium (#130-107-879 (Miltenyi Biotec, Inc., San Diego, Calif., USA)) supplemented with 5% AB serum (alternatively, other media e.g., Life Technologies, R&D systems media, CELLGENIX media, and the like, may be used, with or without the use of human serum)) and 500 IU/mL human Interleukin 2 (IL-2) (Miltenyi Biotec) at a cell seeding concentration of 0.25-0.5×10⁶/ml in 24-well coated plates without additional activation reagents or in 24-well non-coated plates with a combination of 500 ng/ml soluble anti-NKp46 (clone 9E2) and 500 ng/ml anti-CD2 (clone OKT11) (alternatively, other concentrations of the activating agents may be used, and other agents may be added for activation).

2. The activated αβ TCRneg cell population was further culture-expanded in feeder-free culture conditions with BINATE medium and 500 IU/mL IL-2 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days (less frequently for a bioreactor (5-7 days)) with fresh BINATE medium and IL-2. In certain variations of the method in this Example, and other Examples herein, after 1 week, 2 weeks, 3 weeks, 4 weeks, or 5 weeks of expansion, cultured BINATE cells may be cryopreserved. In one example, expanded BINATE cells are washed, then concentrated, and serum-free cryopreservation media (BIOLIFE) is added. The cryopreserved BINATE product may thawed and seeded again as described herein for further culture, or may be thawed and infused into a patient with cancer or viral infection, for example.

3. At the end of day 15 expansion, BINATE cells were specifically analyzed for NK and γδ CD3+ T cell content and their subsets, such as CD56, as well as additional analysis of potential contaminating cell types, such as αβ TCR+ T cells.

The phenotype of BINATE cells, reported as a percentage of total cell population, showed no difference between activation on coated plates or with soluble antibodies. The NK population (CD56+CD3-) was 89.1% of the final population when activated on coated plates compared to 92% when activated with soluble antibodies. CD3+γδT cells were 6.2% and 5.5% of the population in the coated plates versus soluble conditions, respectively. CD3+CD56+γδT cells and CD3+CD56− γδT cells were 2.8% and 3.4% of the population in the coated plate activated cultures, respectively, and 3.3% and 2.2% of the population in the soluble activation cultures, respectively. The percent of αβTCR+CD3+ T cells was negligible in both conditions at 0.12% for the coated activation and 0.41% for the soluble activation.

While historical reports have shown that the use of crosslinked antibodies might have greater activity, the use of soluble antibodies often is preferred to crosslinked (such as used on surfaces such as plates or beads) due to greater control of the culture, for example. There was no difference in the use in the combination of the soluble anti-NKp46 (clone 9E2, Miltenyi) and soluble anti-CD2 (clone OKT11) compared to the crosslinked condition, and all experiments subsequently were performed with soluble antibody activation reagents.

### Example 8: Evaluation of Activation and Expansion Using Varying Amounts of Anti-NKp46

In a separate experiment, a different anti-NKp46 (Bab281) reagent was evaluated at 3 concentrations (10 ng/ml, 50 ng/ml and 500 ng/ml) in two expansion conditions: 1) IL-2 (500 IU/mL human Interleukin 2 (Miltenyi Biotec)) or 2) IL-15 (10 ng/ml human interleukin 15 (Miltenyi Biotec)).

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and IL-2 or IL-15 at a cell seeding concentration of 0.25-0.5×10⁶/ml in a 24 well plate with a combination of 500 ng/ml anti-CD2 (clone OKT11) and one of 3 concentrations of anti-NKp46 (Bab281): 10 ng/ml, 50 ng/ml or 500 ng/ml.

2. The activated αβ TCRneg cell population was further culture-expanded in feeder-free culture conditions with BINATE medium and 500 IU/mL IL-2 or 10 ng/ml IL-15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2 or IL-15.

3. At the end of day 15 expansion BINATE cells were analyzed for NK and γδ CD3+ T cell content and their subsets, such as CD56, as well as additional analysis of potential contaminating cell types, such as αβ TCR+ T cells.

The phenotype of BINATE cells, reported as a percentage of total cell population, showed no significant difference among the 3 concentrations of anti-NKp46 (Bab281) when culture expanded with IL-15 expansion. The NK population (CD56+CD3−) constituted 84.5%, 87% and 87.2% with 500 ng/ml anti-NKp46 (Bab281), 50 ng/ml anti-NKp46 (Bab281), and 10 ng/ml anti-NKp46 (Bab281), respectively. The γδTCR CD3+ population constituted 11.4%, 8.7%, and 8.1%, respectively, while the CD3+56+ population constituted 7.40%, 5.40% and 5.30%, respectively. The αβTCR+ cells were virtually undetectable at all concentrations.

TABLE 1

| Total % BINATE | CD56+ | | CD3+ | CD3+ | αβ |
|---|---|---|---|---|---|
| Phenotype | CD3− | CD3+ | CD56+ | CD56− | TCR+ |
| Bab281 500 ng/OKT11 | 84.50 | 11.40 | 7.40 | 4.00 | 0.00 |
| 500 ng/IL-15 | | | | | |
| Bab281 50 ng/OKT11 | 87.00 | 8.70 | 5.40 | 3.30 | 0.01 |
| 500 ng/IL-15 | | | | | |
| Bab281 10 ng/OKT11 | 87.20 | 8.10 | 5.30 | 2.80 | 0.01 |
| 500 ng/IL-15 | | | | | |
| Bab281 500 ng/OKT11 | 85.70 | 4.40 | 2.90 | 1.40 | 0.00 |
| 500 ng/IL-2 | | | | | |
| Bab281 50 ng/OKT11 | 82.80 | 4.50 | 3.90 | 0.60 | 0.00 |
| 500 ng/IL-2 | | | | | |
| Bab281 10 ng/OKT11 | 82.40 | 4.80 | 4.00 | 0.80 | 0.09 |
| 500 ng/IL-2 | | | | | |

### Example 9: Marker Evaluation of Activated and Expanded Cells

In a separate experiment, the BINATE phenotype after activation with the combination of 10 ng/ml Good Manufacturing Practice (GMP) quality anti-NKp46 (Bab281) antibody and 500 ng/ml GMP quality anti-CD2 (OKT11) antibody in two expansion conditions (i.e., IL-2 and IL-15) was compared.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and either 500 IU/ml IL-2 or 10 ng/ml IL-15 at a cell seeding concentration of 0.25-0.5×10⁶/ml in a 24 well plate with 500 ng/ml of anti-CD2 (OKT11) and 10 ng/ml anti-NKp46 (Bab281) (alternatively, other concentrations of the activating agents may be used, and other agents may be added for activation).

2. The activated αβ TCRneg cell population was further culture-expanded in feeder-free culture conditions with BINATE medium and 500 IU/mL IL-2 or 10 ng/ml IL15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2 or IL-15.

3. At the end of a 20 day culture expansion BINATE cells were specifically analyzed for markers of innate, cytotoxic and exhaustion expression cell content and their subsets, such as CD56, CD16 and CD57 as well as additional analysis of potential contaminating cell types, such as αβ TCR+ T cells.

The total BINATE cell population contained 92% and 84.3% CD56+ innate cells after IL-15 and IL-2 expansion, respectively. The population contained 33% and 12% of CD56+CD16+ cytotoxic innate cells, 76% and 59.6% CD8+ cytotoxic innate cells, and 99% and 96.5% CD56+CD57− immature innate cells after IL-15 and IL-2 culture expansion, respectively. There were no detectable abTCR+ T cells after either IL-15 or IL-2 culture expansion. CD4+, CD8+, and CD4-CD8− populations of NK and γδ T cells are provided in Table 2 below.

TABLE 2

| | CD4+ | CD8+ | CD4−CD8− |
|---|---|---|---|
| NK IL-15 | 0 | 77.9 | 22.1 |
| γδT cell IL-15 | 1.2 | 20.7 | 76.1 |
| NK IL-2 | 0 | 72.2 | 27.8 |
| γδT cell IL-2 | 1.5 | 35.17 | 61.36 |

### Example 10: Evaluation of Cells Expanded Under Static and Bioreactor Culture Conditions

In a separate experiment, the BINATE proliferation in static and bioreactor culture conditions after activation with the combination of 50 ng/ml GMP quality anti-NKp46 (Bab281) antibody and 50 ng/ml GMP quality anti-CD2 (OKT11) antibody in two expansion conditions (IL-2 and IL-15) was compared.

1. A buffy coat from a normal healthy donor underwent gradient cell separation with Ficoll® performed with manual separation in a centrifuge. Mononuclear cells were subjected to αβ T cell depletion using a research scale Miltenyi LS-column separation. The resulting αβ TCRneg cell population was placed in feeder-free culture conditions with BINATE medium and either 500 IU/ml IL-2 or 10 ng/ml IL-15 at a cell seeding concentration of 0.25-0.5×10⁶/ml in a 24 well plate with a combination of 50 ng/ml of anti-CD2 (OKT11) and 50 ng/ml anti-NKp46 (Bab281) (alternatively, other concentrations of the activating agents may be used, and other agents may be added for activation).

2. The activated αβ TCRneg cell population was further culture-expanded in feeder-free culture conditions with BINATE medium and 500 IU/mL IL-2 or 10 ng/ml IL-15 at a cell seeding concentration of 0.25-0.5×10⁶/ml in T75 flasks or in a G-Rex benchtop bioreactor (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-15.

3. The activated αβ TCRneg cell population was also further culture-expanded in feeder-free culture conditions in the GRex culture device with BINATE medium and 10 ng/ml IL-15 at a cell seeding concentration of 1×10⁶/ml. Media exchanges took place every 7 days with fresh BINATE medium and IL-15.

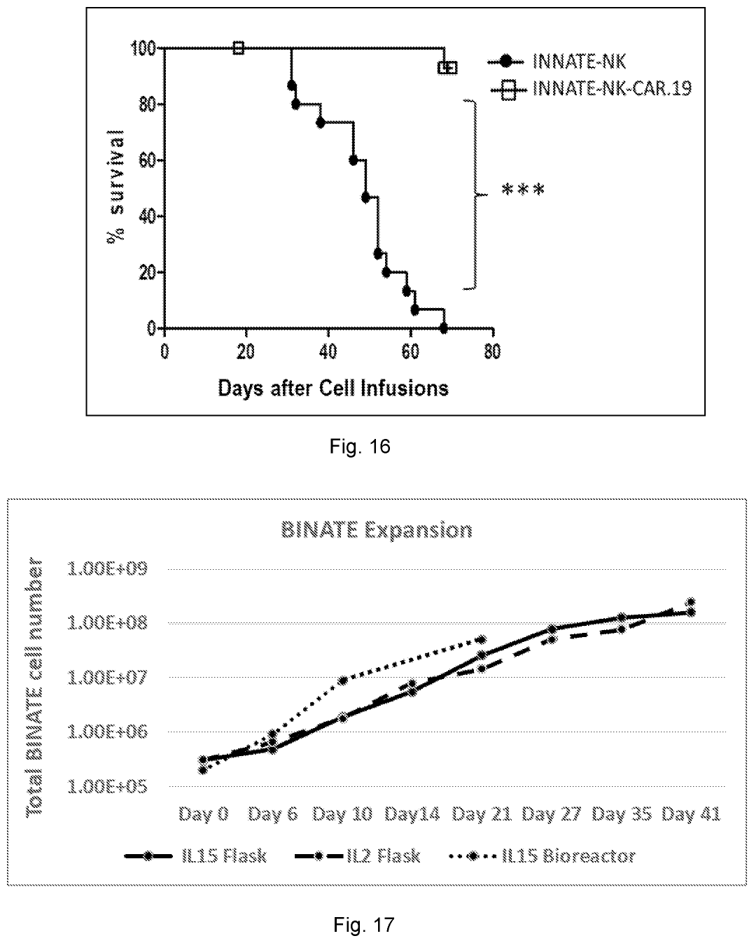

Total BINATE expansion is shown as total cell number in FIG. 17. Activated αβ TCRneg cell expansion in flasks in either IL-2 or IL-15 supplemented BINATE medium was equivalent over a 41 day period. Similarly, IL-15 expansion of the activated αβ TCRneg cell population in the bioreactor over the first 21 days was equivalent to that of IL-15 expansion in flasks.

### Example 11: Evaluation of Alloreactivity of Activated and Expanded Cells

In a separate experiment, the alloreactivity of the activated BINATE cell population expanded in either IL-2 or IL-15 was evaluated.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium at a cell seeding concentration of 0.25-0.5×10⁶/ml in a 24 well plate with a combination of 50 ng/ml of anti-CD2 (OKT11) and 50 ng/ml anti-NKp46 (Bab281) (alternatively, other concentrations of the activating agents may be used, and other agents may be added for activation).

2. The activated αβ TCRneg cell population was further culture-expanded in feeder-free culture conditions with BINATE medium and either 500 IU/mL IL-2 or 10 ng/ml IL-15 at a cell seeding concentration of 0.25-0.5×10⁶/ml in T75 flasks. Media exchanges took place every 3 days for the flasks with fresh BINATE medium and IL-2 or IL-15.

3. BINATE cells and stimulator cells derived from PBMC of 3 random healthy donors (HD) were tested in one-way mixed lymphocyte reactions (MLR). Stimulator cells from three HD were pooled and irradiated (30 Gy) before use. BINATE cells were seeded at 100 μl/well (1×10⁵ cells) with an equal number of stimulator cells in 200 μl in 96-well in BINATE Media, flat bottom microtiter plates. After a 5-day incubation, cultures were pulsed with 18 kBq ³H-thymidine for 12 h and harvested on glass fiber filters. Dry filters were counted on a Microbeta Trilux 1450 counter (Wallac, Perkin Elmer). Results were expressed as SI (cpm with antigen/cpm background). BINATE cells without cytokine addition were used as negative control, while positive control was represented by BINATE cells seeded with cytokine (IL-2 or IL-15).

The mixed lymphocyte co-cultures were analyzed for ³H thymidine incorporation (CPM), an indication of proliferation. In the IL-15 experiment, the negative control consisted of unstimulated BINATE cells without IL-15 supplementation and had a ³H thymidine uptake of 1144, indicating essentially no proliferation. The irradiated pooled healthy donor cells (HD) had a ³H thymidine uptake of 1194, also indicating no proliferation. The positive control included proliferation stimulation with IL-15 and had a ³H thymidine uptake of 15558, a 14-fold increase over the negative control. The activated BINATE cells which had undergone expansion in IL-15 had a ³H thymidine uptake of 942, essentially no difference from background.

In the IL-2 experiment, the negative control included unstimulated BINATE cells without IL-2 supplementation and had a ³H thymidine uptake of 834, indicating essentially no proliferation. The irradiated pooled healthy donor cells (HD) had a ³H thymidine uptake of 1194, also indicating no proliferation. The positive control included proliferation stimulation with IL-2 and had a ³H thymidine uptake of 18834, a 22-fold increase over the negative control. The activated BINATE cells which had undergone expansion in IL-15 had a ³H thymidine uptake of 1245, essentially no difference from the background controls.

The absence of an MLR response may be due to the absence of αβ TCR+ T cells, and supports the lack of alloreactivity in the innate BINATE cell population. The absence of alloreactivity is important clinically because it supports the lack of potential for the cells to result in a graft versus host response.

### Example 12: Evaluation of Cell Proliferation and Phenotype Under Different Expansion Conditions

In a separate experiment, the proliferation capability and BINATE phenotype after activation with GMP quality anti-NKp46 (Bab281) antibody (50 ng/ml) (Caprico) and GMP quality anti-CD2 (OKT11) antibody (50 ng/ml) (Caprico) in 5 different expansion conditions were compared.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions with BINATE medium with a combination of 50 ng/ml of anti-CD2 (OKT11) and 50 ng/ml anti-NKp46 (Bab281) (alternatively, other concentrations of the activating agents may be used, and other agents may be added for activation), at a cell seeding concentration of 0.25-0.5×10⁶/ml in a 24 well plate and one of the following expansion supplements:
a. 500 IU/mL IL-2 (Miltenyi Biotec)
b. 10 ng/ml IL-15 (Miltenyi Biotec)
c. combination of 500 IU/mL IL-2 and IL-15 10 ng/mL (IL-2/IL-15)
d. combination of 500 IU/mL IL-2 and 10 ng/ml OKT3 clone anti-CD3 antibody (Miltenyi Biotec) (IL-2/OKT3)
e. combination of IL-15 10 ng/mL and 500 IU/mL IL-2 and 10 ng/ml OKT3 (IL-2/IL-15/OKT3).

2. The activated αβ TCRneg cell population was further culture-expanded in feeder-free culture conditions with BINATE medium at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)) and one of the five expansion conditions listed above. Media exchanges took place every 3 days for the plates or flasks (less frequently for a bioreactor (5-7 days)) with fresh BINATE medium and the same expansion condition used during the initial culturing activation and expansion phase.

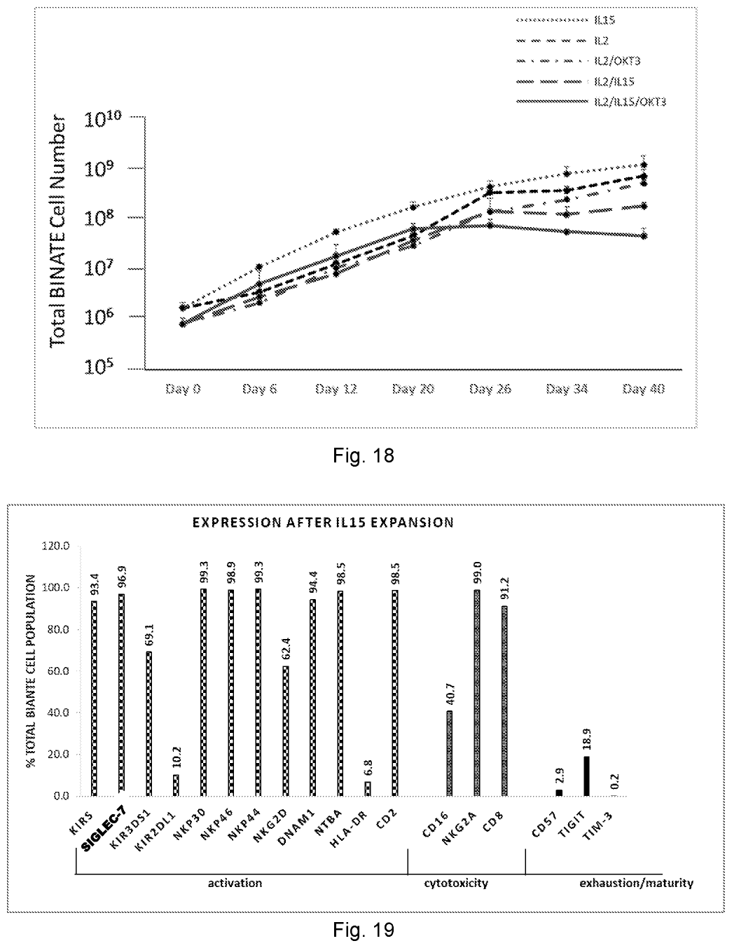

Total BINATE expansion is shown as total cell number in FIG. 18. Activated el TCRneg cell expansion in flasks in either IL-2, IL- or IL-2/OKT3 supplemented BINATE medium was equivalent over a 40 day period. Activated αβ TCRneg cell expansion in flasks in either IL-2/IL-15 or IL-2/IL-15/OKT3 supplemented BINATE medium was equivalent over a26 day period and then proliferation was slowed and overall cell numbers were less compared to the other 3 groups from day 26 to day 41 in culture.

As shown in Table 3 below, on days 7, 20 and 30 of culture expansion, BINATE cells from each of the 5 conditions were determined by flow cytometry to be NK or γδT cells. As shown in Table 4, the cells from each of the conditions at days 7, 20 and 30 were also analyzed by flow cytometry for specific markers of innate, cytotoxic and exhaustion and immaturity expression on the cells as well as the specific VdTCR usage in the γδT cells.

TABLE 3

| | | | | CD3+ |
|---|---|---|---|---|
| | CD56+CD3− | CD3+CD56+ | CD3+CD56− | Total |
| | NK | γδT cell | γδT cell | γδT cell |
| IL-15 | | | | |
| Day 7 | 56.25 | 21.65 | 14.55 | 36.2 |
| Day 20 | 83.45 | 12.55 | 3.7 | 16.25 |
| Day 30 | 99.3 | 0.6 | 0.1 | 1.0 |
| IL-2 | | | | |
| Day 7 | 62.4 | 11.85 | 12.55 | 24.4 |
| Day 20 | 56.55 | 22.95 | 15.3 | 38.25 |
| Day 30 | 85.95 | 12.65 | 1.15 | 13.8 |
| IL-2/IL-15 | | | | |
| Day 7 | 52.4 | 24.15 | 15.2 | 39.35 |
| Day 20 | 51.7 | 36.85 | 11 | 47.85 |
| Day 30 | 49.15 | 49.55 | 1.15 | 50.7 |
| IL-2/OKT3 | | | | |
| Day 7 | 29.15 | 9.4 | 42.55 | 51.95 |
| Day 20 | 26.65 | 26.3 | 43.25 | 69.55 |
| Day 30 | 58.9 | 29.2 | 5.55 | 34.75 |
| IL-2/IL-15/ | | | | |
| OKT3 | | | | |
| Day 7 | 29.8 | 26.3 | 43.95 | 70.25 |
| Day 20 | 36.6 | 47.5 | 15.35 | 62.85 |
| Day 30 | 53.6 | 44.15 | 2.15 | 46.3 |

TABLE 4

| | | | | | | | | | % Vd1- | | | % Vd1- |
|---|---|---|---|---|---|---|---|---|---|---|---|---|
| | % CD16+ | % CD57+ | % CD16+ | % CD57+ | % CD16+ | % CD57+ | % Vd1 | % Vd2+ | Vd2− of | % Vd1 | % Vd2+ | Vd2− of |
| | of CD56+ | of CD56+ | of CD3+ | of CD3+ | of CD3+ | of CD3+ | of CD3+ | of CD3+ | CD3+ | of CD3+ | of CD3+ | CD3+ |
| Expansion | CD3− | CD3− | CD56+ | CD56+ | CD56− | CD56− | CD56+ | CD56+ | CD56+ | CD56− | CD56− | CD56− |
| Condition | Cells | Cells | Cells | Cells | Cells | Cells | Cells | Cells | Cells | Cells | Cells | Cells |
| IL-15 | | | | | | | | | | | | |
| Day 7 | 54.75 | 25.30 | 0.60 | 27.10 | 2.70 | 24.05 | 10.15 | 87.30 | 2.50 | 30.55 | 66.70 | 2.60 |
| Day 20 | 43.45 | 6.85 | 26.45 | 30.80 | 2.60 | 10.60 | 20.00 | 67.80 | 11.85 | 11.90 | 84.10 | 2.50 |
| Day 30 | 27.95 | 5.55 | 23.50 | 18.70 | 1.95 | 10.60 | 17.10 | 27.15 | 5.65 | 9.60 | 36.05 | 3.85 |
| IL-2 | | | | | | | | | | | | |
| Day 7 | 52.75 | 29.65 | 3.00 | 48.10 | 1.60 | 34.85 | 42.60 | 47.75 | 8.95 | 52.95 | 39.30 | 7.60 |
| Day 20 | 46.50 | 15.20 | 22.90 | 31.40 | 3.85 | 17.95 | 85.15 | 4.15 | 9.45 | 76.80 | 16.30 | 6.65 |
| Day 30 | 17.10 | 5.65 | 24.15 | 36.80 | 3.05 | 24.00 | 72.20 | 9.65 | 19.40 | 73.35 | 14.80 | 11.50 |
| IL-2/IL-15 | | | | | | | | | | | | |
| Day 7 | 55.75 | 27.10 | 5.40 | 41.80 | 0.00 | 8.60 | 28.00 | 69.30 | 2.35 | 20.05 | 76.55 | 3.50 |
| Day 20 | 56.60 | 14.35 | 35.85 | 22.15 | 12.55 | 22.05 | 52.20 | 41.60 | 5.35 | 35.65 | 54.40 | 9.20 |
| Day 30 | 33.35 | 26.55 | 21.70 | 37.65 | 1.75 | 11.70 | 82.50 | 9.80 | 7.20 | 71.45 | 16.40 | 11.70 |
| IL-2/OKT3 | | | | | | | | | | | | |
| Day 7 | 57.70 | 31.95 | 1.65 | 24.50 | 0.00 | 7.90 | 62.15 | 26.65 | 9.80 | 42.95 | 37.45 | 19.30 |
| Day 20 | 55.40 | 14.55 | 29.05 | 27.70 | 4.10 | 14.00 | 81.40 | 5.75 | 13.80 | 72.75 | 14.40 | 10.75 |
| Day 30 | 43.25 | 25.15 | 27.65 | 34.70 | 1.95 | 21.15 | 70.55 | 17.15 | 6.05 | 52.25 | 34.90 | 12.15 |
| IL-2/IL-15/ | | | | | | | | | | | | |
| OKT3 | | | | | | | | | | | | |
| Day 7 | 53.85 | 29.80 | 2.45 | 31.60 | 0.00 | 4.05 | 42.55 | 46.90 | 9.80 | 44.50 | 38.10 | 17.25 |
| Day 20 | 48.70 | 17.65 | 12.25 | 17.80 | 13.00 | 16.55 | 49.05 | 32.60 | 16.70 | 43.35 | 38.75 | 15.40 |
| Day 30 | 31.40 | 29.90 | 14.70 | 22.85 | 2.55 | 19.15 | 54.30 | 29.20 | 16.10 | 50.25 | 26.25 | 22.60 |

PD-1 expression, an indicator of cell exhaustion, was shown to be low. On day 30, the IL-15 expansion conditions showed 2.7% expression in the total BINATE population with 2% expression on CD56+CD3− NK cells, 10.6% expression on CD3+CD56− γδT cells and 17.05% expression on CD3+CD56+γδT cells, demonstrating a lack of exhaustion. Under IL-2 expansion conditions, PD-1 expression was shown to be low at 14.2% of the total BINATE population, as determined by flow cytometry in the IL-2 expansion condition, with CD56+CD3− NK cells expressing 2% PD-1, and CD3+CD56− γδT cells and CD3+CD56+γδT cells expressing 62.9% and 78.3%, respectively.

### Example 13: CD107a Cytotoxicity

Day 30 cells from each of the 5 expansion conditions in Example 12 were co-cultured with tumor cell lines, including IMR32, SH-SY5Y (neuroblastoma cell lines) and K562 (B cell leukemia cell line). After 3 days in culture, the expression of CD107a, a measurement of cytotoxic granule release was measured on basal BINATE cells and on BINATE cells co-cultured with the respective tumor cell line by means of flow cytometric analysis. Table 5 shows the difference between the tumor-stimulated expression minus the basal value for each tumor cell line and for each BINATE subset (CD56+CD3− NK cells, and CD3+CD56+γδT cells, and CD3+CD56− γδT cells) in each of the 5 expansion conditions (IL-2, IL-2/OKT3, IL-15, IL-2/IL-15 and IL-2/IL-15/OKT3). The statistical significance is reported as the p value from a T-test.

TABLE 5

| | IMR32 | | SH-SY5Y | | K562 | |
|---|---|---|---|---|---|---|
| | minus | P | minus | P | minus | P |
| | Basal | Value | Basal | Value | Basal | Value |
| CD56+CD3− NK | | | | | | |
| IL-2 | 8.60 | 0.042 | 29.30 | 0.069 | 48.55 | 0.023 |
| IL-2/OKT3 | 6.60 | 0.022 | 40.55 | 0.025 | 57.05 | 0.005 |
| IL-15 | 29.60 | 0.103 | 35.10 | 0.050 | 54.90 | 0.049 |
| IL-2/IL-15 | 8.25 | 0.058 | 25.90 | 0.122 | 50.55 | 0.045 |
| IL-2/IL-15/OKT3 | 3.70 | 0.020 | 45.35 | 0.037 | 47.55 | 0.079 |
| CD3+CD56+ γδT | | | | | | |
| IL-2 | 22.05 | 0.015 | 21.05 | 0.066 | 25.05 | 0.045 |
| IL-2/OKT3 | 30.75 | 0.227 | 44.40 | 0.057 | 35.75 | 0.259 |
| IL-15 | 23.45 | 0.127 | 45.20 | 0.033 | 57.55 | 0.039 |
| IL-2/IL-15 | 26.90 | 0.102 | 14.90 | 0.321 | 28.35 | 0.024 |
| IL-2/IL-15/OKT3 | 33.15 | 0.068 | 22.70 | 0.184 | 35.25 | 0.199 |
| CD3+CD56− γδT | | | | | | |
| IL-2 | 1.35 | 0.012 | −2.70 | 0.024 | 2.85 | 0.007 |
| IL-2/OKT3 | −1.70 | 0.259 | 5.05 | 0.296 | 3.05 | 0.405 |
| IL-15 | 4.15 | 0.14 | 22.90 | 0.192 | 8.90 | 0.088 |
| IL-2/IL-15 | −7.05 | 0.277 | −4.25 | 0.264 | −1.20 | 0.433 |
| IL-2/IL-15/OKT3 | −0.20 | 0.443 | 3.05 | 0.278 | 9.70 | 0.345 |

### Example 14: Evaluation of Phenotype Markers Under Different Expansion Conditions

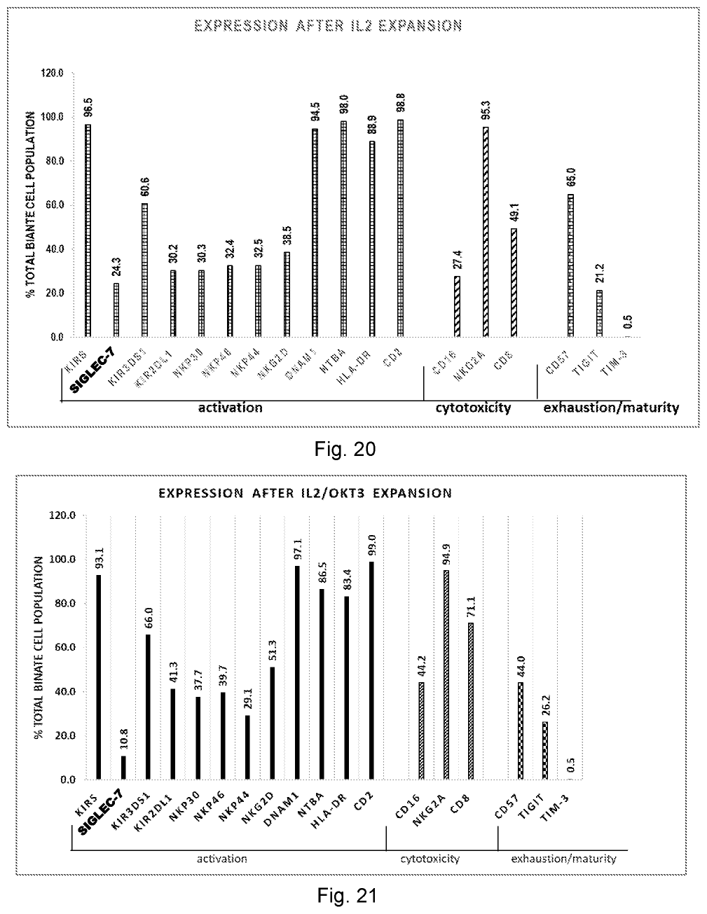

Day 20 cells from 3 expansion conditions in Example 12 (conditions IL-2, IL-15 and IL-2/OKT3) were analyzed for specific activation, cytotoxic and exhaustion markers. FIG. 19 shows a phenotypic analysis by marker type (activation, cytotoxicity and exhaustion/immaturity) for the IL-15 expansion condition. FIG. 20 shows a phenotypic analysis by marker type (activation, cytotoxicity and exhaustion/immaturity) for the IL-2 expansion condition. FIG. 21 shows a phenotypic analysis by marker type (activation, cytotoxicity and exhaustion/immaturity) for the IL-2/OKT3 expansion condition. Under all conditions, BINATE cells generally showed high activation and cytotoxicity and low exhaustion.

### Example 15: Evaluation of In Vitro Tumor Killing

In a separate experiment, in vitro tumor killing of BINATE cells after activation with a combination of 10 ng/ml anti-NKp46 (Bab281) antibody and 500 ng/ml anti-CD2 (OKT11) antibody in two expansion conditions (IL-2 and IL-15) was compared.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and either 500 IU/ml IL-2 or 10 ng/ml IL-15 at a cell seeding concentration of 0.25-0.5×10⁶/ml in a 24 well plate with a combinations of 500 ng/ml of anti-CD2 (OKT11) and 10 ng/ml anti-NKp46 (Bab281) (alternatively, other concentrations of the activating agents may be used, and other agents may be added for activation).

2. The activated αβ TCRneg cell population was further culture-expanded in feeder-free culture conditions with BINATE medium and 500 IU/mL IL-2 or 10 ng/ml IL-15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2 or IL-15.

3. At the end of a 20-day culture expansion BINATE cells were placed in tumor co-culture experiments for 5-7 days. The experiment shown in Table 6 below involved culturing leukemia cell lines at a 1:1 effector to target ratio (i.e., 0.5 M BINATE cells (effectors) with 0.5 M tumor cells (targets)) in 2 ml media/culture well in a 6 well culture dish.

The experiment shown in Table 7 below involved culturing solid tumor cell lines (neuroblastoma, sarcoma and colon tumor lines) at a 2:1 effector to target ratio (i.e., 0.5 M BINATE cells (effectors) with 0.25 M tumor cells (targets)) in 2 ml media/culture well in a 6 well culture dish. Co-cultures of tumor and effector cells were performed in the absence of cytokines. Co-cultures were evaluated on days 3-7 by microscopy and flow cytometry.

In examining residual tumor in a co-culture experiment, Table 6 shows that activated BINATE cells expanded in either IL-2 or IL-15 were successful in eradicating 50% or greater tumor cells of all cell lines, and more than 75% in 4-5 of the AML cell lines. In Table 6, short term co-culture of all cell lines resulted in BINATE cells killing all 221 leukemia cells, >80% Daudi leukemia cells, and up to 50% of the Karpas cell line, which is often considered resistant to innate killing.

TABLE 6
Residual Tumor Cells after Co-Culture with BINATE Cells

| | IL-2 | IL-15 |
|---|---|---|
| | Expansion | Expansion |
| Acute myelocytic leukemia cell lines | | |
| OCI-AML | 2.3 | 0.8 |
| 697 | 16.7 | 9.4 |
| MV4: 11 | 4.2 | 22.8 |
| RS4; 11 | 35.5 | 54.2 |
| Molm-1 | 1 | 22.8 |
| THP1 | 0.5 | 1.1 |
| Acute lymphoblastic leukemia cell lines | | |
| Daudi | 5.10 | 9.3 |
| 221 | 1.05 | 0.85 |
| Karpas | 87.6 | 71.4 |

In examining residual tumor in a co-culture experiment, Table 7 shows that activated BINATE cells expanded in either IL-2 or IL-15 were successful in eradicating neuroblastoma tumors in short term co-culture experiments. IL-2 expanded BINATE cells were not tested in co-culture with colon cancers, while IL-15 expanded BINATE cells showed activity against sarcoma and colon cancers.

TABLE 7
Residual Tumor Cells after Co-Culture with BINATE Cells

| | BINATE IL-15 | BINATE IL-2 |
|---|---|---|
| Neuroblastoma | | |
| SH-SY5Y | 0.9 | 4.5 |
| IMR32 | 0.2 | 18.7 |
| SKNSH | 18.5 | 27.05 |
| CHLA | 3.85 | 14.75 |
| LAN-1 | 6.5 | 10.8 |
| SKNSKBeC | 6.8 | 35.6 |
| Sarcoma | | |
| RD | 1.7 | 15.3 |
| A673 | 0.6 | 0.7 |
| CT10 | 0.2 | 0.7 |
| G401 | 0.2 | ND* |
| Colon | | |
| SW480 | 4.6 | ND |
| DLD1 | 8.3 | ND |

*Not Determined

### Example 16: Evaluation of In Vitro Transduction Efficiencies

In a separate experiment, to evaluate stability of transduction early and later in vitro transduction efficiencies of BINATE cells were compared after activation with a combination of 10 ng/ml anti-NKp46 (Bab281) antibody and 500 ng/ml anti-CD2 (OKT11) antibody with a retroviral construct encoding a third generation GD2 chimeric antigen receptor (CAR).

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and 500 IU/ml IL-2 at a cell seeding concentration of 0.25-0.5×10⁶/ml in a 24 well plate with a combination of 500 ng/ml of anti-CD2 (OKT11) and 10 ng/ml anti-NKp46 (Bab281) (alternatively, other concentrations of the activating agents may be used, and other agents may be added for activation).

2. For generation of any BINATE.CAR described herein, an aliquot of cells from the activated culture-expanded αβ TCRneg cell population was taken on day 4-15 and seeded onto a human fibronectin-coated plate or alternatively a culture bag (Gobain)) at the concentration of 0.25×10⁶/ml in the presence of gamma retroviral supernatant containing the construct for the specific chimeric antigen receptor. After three to four days, the cells are washed and the process continues as for non-transduced cells, which may include either expansion or cryopreservation. Expansion conditions may contain 500 IU/mL human Interleukin 2 (IL-2) or 10 ng/ml IL-15, but may contain any of the condition combinations mentioned herein. The chimeric antigen receptor (CAR) constructs used herein include CAR.GD2, CAR.123, and CAR.CD19 which contain nonfunctional markers, such as a fragment of the CD34 surface antigen or a mutant CD19 fragment (see Example 25 for descriptions of certain constructs). In certain variations of the methods herein, VECTOFUSION-1 (Miltenyi Biotec) may be used to assist in retroviral transfection instead of RETRONECTIN, and genetic modification may include gene modification of other CAR constructs of any antigen of choice or modification with other molecules. For this Example, on day 5 activated αβ TCRneg cell population was placed onto a human fibronectin-coated plate (or alternatively a culture bag (Gobain)) at a concentration of 0.25×10⁶/ml in the presence of gamma retroviral supernatant containing a construct for the GD2 chimeric antigen receptor (CAR). The GD2 construct contained a mutant fragment of the CD34 receptor as a selection and tracking marker.

3. After three days, the transduced cells were washed and further culture-expanded in feeder-free culture conditions with BINATE medium and 500 IU/mL IL-2 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2.

4. On day 3 and day 15 after transduction, the BINATE cell subsets, as well as the VdTCR subsets of the γδT cells were analyzed for expression of the mutant CD34 receptor (an indicator of transduction efficiency). On days 3 and 15, transduction efficiency of the CD56+CD3− NK cells was 82.4% and 82.4%, respectively. On days 3 and 15, transduction efficiency of the CD3+56+ cells was 43.2% and 62.4%, respectively. On days 3 and 15, transduction efficiency of the CD3+CD56− γδT cells was 37.3% and 56.8%, respectively. Genetic modification was stable over culture time, signifying a highly activated population enabling high transduction and permanent integration.

In a similar experiment, transduction of BINATE subpopulations showed high transduction with the GD2 CAR construct and polyclonal transduction of both Vd1 and Vd2 TCR γδT cells. The CD56+CD3− NK cells were transduced at 69%, while the CD3+γδT cells were transduced at 39%. The Vd1 TCR population had a transduction efficiency of 67% and the Vd2 TCR population had a transduction efficiency of 34%.

### Example 17: Evaluation of Proliferation after In Vitro Transduction of GD2 CAR

In a separate experiment, the proliferation capability after in vitro transduction of BINATE cells with a retroviral construct encoding a third generation GD2 CAR after activation with the combination of 50 ng/ml anti-NKp46 (Bab281) antibody and 50 ng/ml anti-CD2 (OKT11) antibody was compared to unmodified BINATE cells in IL-2 and IL-15 expansion conditions.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and 500 IU/ml IL-2 or 10 ng/ml IL-15 at a cell seeding concentration of 0.25-0.5×10⁶/ml in a 24 well plate with a combination of 50 ng/ml of anti-CD2 (OKT11) and 50 ng/ml anti-NKp46 (Bab281) (alternatively, other concentrations of the activating agents may be used, and other agents may be added for activation).

2. The activated non-genetically modified αβ TCRneg cell population was further culture-expanded in feeder-free culture conditions with BINATE medium and 500 IU/mL IL-2 or 10 ng/ml IL-15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2 or IL-15.

3. On day 5 activated αβ TCRneg cell population was placed onto a human fibronectin-coated plate or culture bag (Gobain) at the concentration of 0.25×10⁶/ml in the presence of gamma retroviral supernatant containing the construct for the GD2 chimeric antigen receptor (CAR). The GD2 construct contained a mutant fragment of the CD34 receptor as a selection and tracking marker.

4. After 5 days, the transduced cells were washed and further culture-expanded in feeder-free culture conditions with BINATE medium and 500 IU/mL IL-2 or 10-ng/ml IL-15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2 or IL15.

5. Cell number was recorded for the BINATE and BINATE.CARGD2 populations every 7 days.

Non-modified BINATE cells and BINATE.CARGD2 cells were expanded prior to transduction (represented by the vertical line in FIG. 22). Post transduction expansion is represented by the hatched lines in FIG. 22 for the different conditions. There was no significant difference between genetically modified or unmodified cells, nor was there a difference with IL-2 or IL-15 expansion for the overall cell number.

### Example 18: Evaluation of In Vitro Killing of Solid Tumors

In a separate experiment, in vitro killing of solid tumors after transduction of activated BINATE with a retroviral construct encoding a third generation GD2 CAR in two expansion conditions (IL-2 and IL-15) was compared.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and 500 IU/ml IL-2 or 10 ng/ml IL-15 at a cell seeding concentration of 0.25-0.5×10⁶/ml in a 24 well plate with a combination of 500 ng/ml of anti-CD2 (OKT11) and 10 ng/ml anti-NKp46 (Bab281) (alternatively, other concentrations of the activating agents may be used, and other agents may be added for activation).

2. On day 5 activated αβ TCRneg cell population was placed onto a human fibronectin-coated plate or culture bag (Gobain) at the concentration of 0.25×10⁶/ml in the presence of gamma retroviral supernatant containing the construct for the GD2 chimeric antigen receptor. The GD2 construct contained a mutant fragment of the CD34 receptor as a selection and tracking marker.

3. After three days, the transduced cells were washed and further culture-expanded in feeder-free culture conditions with BINATE medium and 500 IU/mL IL-2 or 10 ng/ml IL-15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2 or IL-15.

4. At the end of a 20 day culture expansion BINATE cells were placed in tumor co-culture experiments for 5-7 days. The experiments involved culturing a 1:1 effector to target ratio (i.e., 0.5 M BINATE cells or BINATE.CAR.GD2 cells (effectors) with 0.5 M Tumor cells (targets)) in 2 ml media/culture well in a 6 well culture dish. Co-cultures of tumor and effector cells were performed in the absence of cytokines. Co-cultures were evaluated on days 3-7 by microscopy and flow cytometry. Table 8 shows data from co-cultures of the percentage of residual tumor after co-culture of the tumor with either unmodified BINATE IL-2 or BINATE IL-15, or CAR.GD2 modified BINATE IL-2 or CAR.GD2 modified BINATE IL-15. Residual tumor in the control was measured at 100%. Both BINATE IL-2 and BINATE IL-15 showed significant tumor killing. The CAR.GD2 modified BINATE IL-2 and BINATE IL-15 showed significant killing as well.

TABLE 8
Residual Tumor Cells after Co-Culture with BINATE Cells and BINATE.CAR.GD2 Cells

| | | BINATE.CARGD2 | | BINATE.CARGD2 |
|---|---|---|---|---|
| | BINATE IL-2 | IL-2 | BINATE IL-15 | IL-15 |
| SH-SY5Y | 47.53 | 41.1 | 8 | 18.85 |
| LAN-1 | 43.5 | 5.5 | 14.7 | 15.9 |
| CHLA | 56.9 | 41.6 | 44.35 | 21.9 |
| IMR-32 | 59.9 | 60.9 | 2.7 | 2.1 |

### Example 19: Evaluation of In Vitro Killing of Myeloid Leukemia Cell Lines

In a separate experiment, in vitro killing of myeloid leukemia cell lines in long term co-cultures after transduction of activated BINATE with a retroviral construct encoding a third generation CD123 CAR was evaluated.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and 10 ng/ml IL-15 at a cell seeding concentration of 0.25-0.5×10⁶/ml in a 24 well plate with a combination of 50 ng/ml of anti-CD2 (OKT11) and 50 ng/ml anti-NKp46 (Bab281) (alternatively, other concentrations of the activating agents may be used, and other agents may be added for activation).

2. On day 5 activated αβ TCRneg cell population was placed onto a human fibronectin-coated plate at the concentration of 0.25×10⁶/ml in the presence of gamma retroviral supernatant containing the construct for the CD123 chimeric antigen receptor (CAR). The CD123 construct contained a mutant fragment of the CD19 receptor as a selection and tracking marker.

3. After three days, the transduced cells were washed and further culture-expanded in feeder-free culture conditions with BINATE medium and 10 ng/ml IL-15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-15.

4. At the end of a 20 day culture expansion BINATE cells and BINATE.CAR123 were placed in tumor co-culture experiments for 7 or more days. The experiments involved culturing a 1:1 effector to target ratio (i.e., 0.5×10⁶ BINATE or BINATE.CAR123 cells (effectors) with 0.5×10⁶ tumor cells (targets)) in 2 ml media/culture well in a 6 well culture dish. Co-cultures of tumor and effector cells were performed in the absence of cytokines. Co-cultures were evaluated on days 3-7 by microscopy and flow cytometry.

The BINATE.CD123 cells were transduced with a 65% efficiency. The myeloid leukemia tumor lines evaluated in co-culture included THP1, MOLM3 and OCI AML cell lines. The data included the percentage of residual tumor after co-culture of the tumor with either unmodified BINATE, or BINATE.CAR.CD123. Residual tumor in the control was measured at 100%. BINATE cells were not successful in eradicating the myeloid leukemia cells due to the continued outgrowth of residual tumor after the initial short term killing. The BINATE.CAR.123 cells were able to successfully kill and maintain a tumor free culture in the co-culture with THP1 and MOLM3 cell lines and >70% of control in the OCI AML cell line.

### Example 20: Evaluation of In Vivo Killing of Leukemia Cells

In a separate experiment, in vivo killing of leukemia cells after transduction of activated BINATE cells with a retroviral construct encoding a second generation CD19 CAR was evaluated.

1. A buffy coat from a normal healthy donor underwent gradient cell separation with Ficoll™ performed with manual separation in a centrifuge. Mononuclear cells were subjected to NK enrichment using a research scale Miltenyi LS-column separation and Miltenyi NK selection kit. The NK and γδT cell population was placed in feeder-free culture conditions, with BINATE medium and 500 IU/ml IL-2 at a cell seeding concentration of 0.25-0.5×10⁶/ml in a 24 well plate with a combination of 500 ng/ml of anti-CD2 antibody (clone LT2, Miltenyi) and 10 ng/ml NKp46 (alternatively, other concentrations of the activating agents may be used, and other agents may be added for activation).

2. On day 5 the activated population was placed onto a human fibronectin-coated plate at the concentration of 0.25×10⁶/ml in the presence of gamma retroviral supernatant containing the construct for the CD19 chimeric antigen receptor. The CD19 construct contained a mutant fragment of the CD34 receptor as a selection and tracking marker.

3. After three days, the transduced cells were washed and further culture-expanded in feeder-free culture conditions with BINATE medium and 500 IU/mL IL-2 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2. At the end of expansion BINATE cells were washed and formulated in PBS solution for IV administration to the mice.

Xenogenic In Vivo Leukemia Models

NOD/SCID IL-2Rγnull (NSG) xenograft mice were infused with DAUDI cells to assess in vivo the antitumor effect of CAR-transduced cells. Mouse experiments were conducted in compliance with the ethical international, EU and national requirements and were approved by the Italian Health Ministry (No 88/2016-PR). NGS mice (5 weeks old; The Jackson Laboratory, USA) were inoculated with Firefly Luciferase-labeled Daudi cells (FF-Daudi) (0.25×10) on day −3. Mice were injected with 50×10 BINATE cells or BINATE.CAR19 cells through IV injection on day 0, and subjected to weekly bioluminescence imaging (IVIS System, Perkin Elmer, USA).

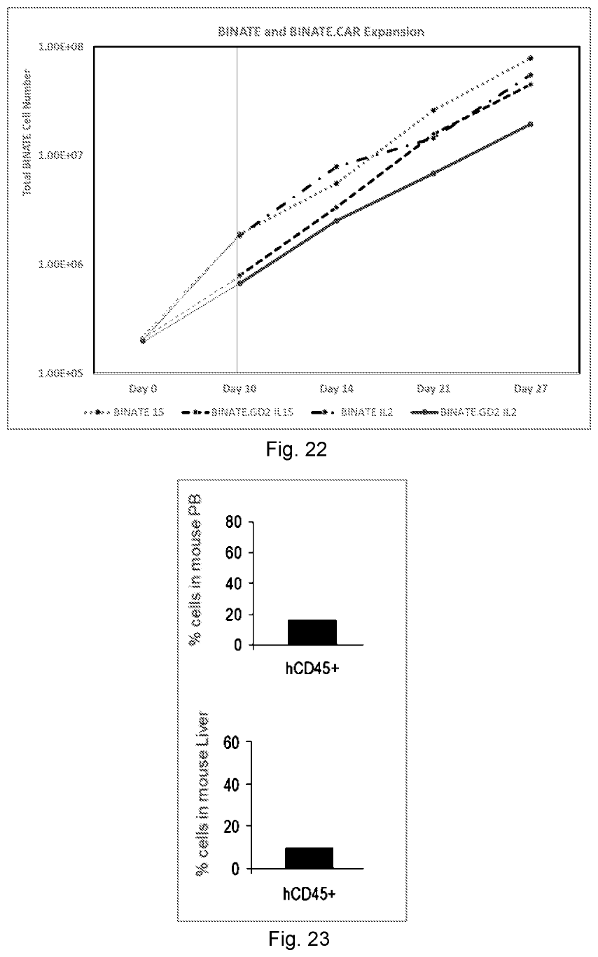

Three doses of BINATE cells or BINATE.CD19 cells were infused into the animal model on day 0. Non-genetically modified BINATE cells were able to give a partial response and mice lived until day 42. Genetically modified BINATE cells with a CAR.CD19 demonstrated a dose effect response with the lowest dose of 1×10⁶ BINATE cells able to sustain the animals one week longer than non-transduced but not as long as the animals who received 5×10⁶ or 10×10⁶ BINATE.CAR.CD19 cells. This model was supported with IL-2 subcutaneous injections twice weekly. Unlike with CAR-T CD19 gene modified αβT cells, the animals lived until they were sacrificed at day 90 and in spite of IL-2 administration, there was no evidence of cytokine release syndrome (CRS), or allo or xeno reactivity, supporting the improved safety of the innate cells. Animals were sacrificed on Day 90 and tissues were evaluated for the presence of human CD45+ cells (BINATE) in the peripheral blood and liver, and then the % NK and γδT subsets were examined in each tissue, respectively. As shown in FIG. 23, there was maintenance of BINATE cells in both blood and tissues, demonstrating the survival and expansion potential of the BINATE cell population in an in vivo milieu.

### Example 21: Evaluation of In Vivo Killing of Solid Tumor (Neuroblastoma)

In a separate experiment, in vivo killing of solid tumor, neuroblastoma, after transduction of activated BINATE cells with a retroviral construct encoding a third generation GD2 CAR was evaluated.

1. A buffy coat from a normal healthy donor underwent gradient cell separation with Ficoll™ performed with manual separation in a centrifuge. Mononuclear cells were subjected to αβ T cell depletion using a research scale Miltenyi LS-column separation. The resulting αβ TCRneg cell population was placed in feeder-free culture conditions, with BINATE medium and 10 ng/ml IL-15 at a cell seeding concentration of 0.25-0.5×10⁶/ml in a 24 well plate with a combination of 50 ng/ml of anti-CD2 (OKT11) and 50 ng/ml anti-NKp46 (Bab281) (alternatively, other concentrations of the activating agents may be used, and other agents may be added for activation).

2. On day 5 the activated αβ TCRneg cell population was placed onto a human fibronectin-coated plate at a concentration of 0.25×10⁶/ml in the presence of gamma retroviral supernatant containing the construct for the GD2 chimeric antigen receptor. The GD2 construct contained a mutant fragment of the CD34 receptor as a selection and tracking marker.

3. After three days, the transduced cells were washed and further culture-expanded in feeder-free culture conditions with BINATE medium and 10 ng/ml IL-15 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days (less frequently for a bioreactor (5-7 days)). On day 20, the cells were washed and formulated in PBS for administration.

Xenogenic In Vivo Neuroblastoma Models

NOD/SCID IL-2Rγnull (NSG) xenograft mice were infused with SH-SY5Y cells to assess in vivo the antitumor effect of CAR-transduced cells. Mouse experiments were conducted in compliance with the ethical international, EU and national requirements and were approved by the Italian Health Ministry (No 88/2016-PR). NGS mice (5 weeks old; The Jackson Laboratory, USA) were inoculated with Firefly Luciferase-labeled SH-SY5Y cells (0.75×10⁸) on day −3 intraperitoneally. Mice were injected with 30×10⁶ BINATE cells or BINATE.CARGD2 cells through intraperitoneal injection every other week for 3 doses, and subjected to weekly bioluminescence imaging (IVIS System, Perkin Elmer, USA).

Non-genetically modified BINATE cells gave a partial response around day 18 with clearance of peripheral tumor bulk, but mice then had localized progression. Genetically modified BINATE cells with CAR.CDGD2 demonstrated significant clearing of peripheral tumor bulk but were not able to kill a local nidus. This model was supported with IL-2 subcutaneous injections twice weekly.

### Example 22: Evaluation of In Vitro Killing of Solid Tumor (Neuroblastoma) and ADCC Antibody by NK Cells

In a separate experiment, in vitro killing of solid tumor, neuroblastoma, by Innate NK cells or Innate NK.CAR.GD2 cells was evaluated.

1. A buffy coat from a normal healthy donor underwent gradient cell separation with Ficoll™ performed with manual separation in a centrifuge. Mononuclear cells were subjected to NK enrichment using a research scale Miltenyi LS-column separation and Miltenyi NK selection kit. The resulting population was placed in feeder-free culture conditions, with BINATE medium and 500 IU/ml IL-2 at a cell seeding concentration of 0.25-0.5×10⁶/ml in a 24 well plate with a combination of 500 ng/ml of anti-CD2 antibody (clone LT2, Miltenyi) and 10 ng/ml NKp46 (alternatively, other concentrations of the activating agents may be used, and other agents may be added for activation).

2. On day 5 the activated population was placed onto a human fibronectin-coated plate at the concentration of 0.25×10⁶/ml in the presence of gamma retroviral supernatant containing the construct for the GD2 chimeric antigen receptor (CAR). The GD2 construct contained a mutant fragment of the CD34 receptor as a selection and tracking marker.

3. After three days, the transduced cells were washed and further culture-expanded in feeder-free culture conditions with BINATE medium and 500 IU/mL IL-2 at a cell seeding concentration of 0.25-0.5×10⁶/ml in T75 flasks (alternatively, cell seeding concentration may range up to 1×10⁶/ml in a culture bag, flask or other large culture vessel such as a Bioreactor (G-Rex)). Media exchanges took place every 3 days for the plates or flasks (less frequently for a bioreactor (5-7 days)) with fresh BINATE medium and IL-2.

4. At the end of a 20 day culture expansion BINATE cells and BINATE.CARGD2 cells were placed in tumor co-culture experiments for 7 or more days. The experiments involved culturing a 1:1 effector to target ratio (i.e., 0.5×10⁶ Innate NK or Innate NK.CAR.GD2 (effectors) with 0.5×10⁶ SH-SY5Y Tumor cells (targets)) in 2 ml media/culture well in a 6 well culture dish. Anti-GD2 antibody (10 μg 14.G2a monoclonal antibody) was added to each of the co-culture groups to evaluate the ADCC potential of the activated cells. Co-cultures of tumor and effector cells were performed in the absence of cytokines. Co-cultures were evaluated on days 3-7 by microscopy and flow cytometry.

The data included the percentage of residual tumor after co-culture of the tumor with unmodified Innate NK, or Innate NK.CAR.GD2, with or without the 14.G2a ADCC antibody. Residual SH-SY5Y tumor in the control was measured at 99.5%. When the 14.G2a antibody was added to the SH-SY5Y tumor cells, there was no difference compared to the control (98.5%). When unmodified activated Innate NK cells were added to the SH-SY5Y tumor cells, the residual tumor was 22%. The addition of 14G.2a antibody to the combination of Innate NK cells and SH-SY5Y tumor cells resulted in less residual tumor percentage (14.5%). Adding Innate NK.CAR.GD2 cells to the SH-SY5Y tumor cells resulted in a residual tumor percentage of 40.9%, and when the Innate NK.CAR.GD2 cells, the SH-SY5Y tumor cells and the 14.G2a antibody were co-cultured, the residual tumor percentage dropped to 8.7%. In the presence of cells with FcγIII receptor expression, such as activated innate cells, ADCC activity with the antibody enhanced tumor killing.

### Example 23

In a separate experiment, effects of switching cytokines during expansion on cell content were evaluated.

1. A buffy coat from a normal healthy donor underwent gradient cell separation with Ficoll™ performed with manual separation in a centrifuge.

2. Mononuclear cells were subjected to αβ T cell depletion using a research scale Miltenyi LS-column separation.

3. The resulting αβ TCRneg cell population was placed in feeder-free culture conditions, with NK MACS™ Medium (#130-107-879 (Miltenyi Biotec, Inc., San Diego, Calif., USA) supplemented with 5% AB serum (alternatively, other media e.g., R&D systems media, CELLGENIX media, and the like, may be used).

4. Activation was achieved with combinations of either 10 ng/ml of anti-NKp46 (clone Bab 281) and 10 ng/ml anti-CD2 (clone OKT11) or 50 ng/ml of anti-NKp46 (clone Bab 281) and 50 ng/ml anti-CD2 (clone OKT11) GMP purified by Caprico (alternatively, other concentrations of the activating agents may be used, and other agents may be added for activation). The activated αβ TCRneg cell population was culture-expanded in feeder-free culture conditions with NK MACS™ Medium (#130-107-879 (Miltenyi Biotec, Inc., San Diego, Calif., USA) supplemented with 5% AB serum (alternatively, other media e.g., R&D systems media, CELLGENIX media, and the like, may be used) at a cell seeding concentration of 0.25×10⁶/ml in a 24 well plate with one of the following conditions:
a. 500 IU/mL human Interleukin 2 (IL-2) (Miltenyi Biotec)
b. 10 ng/ml human Interleukin 15 (IL-15) (Miltenyi Biotec)
c. combination of 500 IU/mL IL-2 and 10 ng/mL IL-15
d. combination of 500 IU/mL IL-2 and 10 ng/ml OKT3 (Miltenyi Biotec)
e. combination of 10 ng/mL IL-15 and 500 IU/mL IL-2 and 10 ng/ml OKT3
   then transferred to a T75 flask.

5. Media exchanges took place every 3 days with fresh medium (less frequently for a bioreactor (5-7 days)) and one of the conditions listed above.

6. Cells were seeded at a concentration of 0.25-0.5×10⁶/ml (alternatively, cell seeding concentration may range up to 1×10⁶/ml in a culture bag, flask or other large culture vessel such as a Bioreactor (G-Rex)).

7. Additionally, cells initially expanded with IL-2 for the first 25 days were then washed and supplemented with IL-15 until the end of culture (day 30). Cells initially expanded with IL-15 were washed on day 25 and then supplemented with IL-2 until the end of culture on day 30 (referred to as cytokine switch conditions). In some instances, cells initially expanded with IL-2 for the first 20 days were then washed and supplemented with IL-15 until the end of culture (day 30). Cells initially expanded with IL-15 were washed on day 20 and then supplemented with IL-2 until the end of culture on day 30. Alternative combinations or other cytokines may be used in the switch conditions such as, for example, IL-7, IL-12, IL-18, IL-21, OKT3, OKT11, and anti-NCRs.

8. At the end of expansion BINATE cells were analyzed for NK and γδ CD3+ T cell content and characterized by receptor expression on the cells via flow cytometry.

In this Example, when the single cytokines were compared to the switch conditions (IL-2 versus IL-2 switch to IL-15), in the case of IL-2 versus IL-2/IL-15, the number of γδT cells were increased upon the addition of IL-15 compared to IL-2 alone (70% versus 50%, respectively). In the case of IL-15 versus IL-15/IL-2, the NK population was increased in the switch condition compared to the IL-15 alone (90% versus 80%, respectively). Since both IL-2 and IL-15 work through interaction with the IL-2 receptor on the cells, the data indicates that the expansion of the population favored in the initial expansion condition with the specific cytokine is potentiated by switching to the other cytokine.

### Example 24: Isolation of Pure NK Cells or Pure γδT Cells

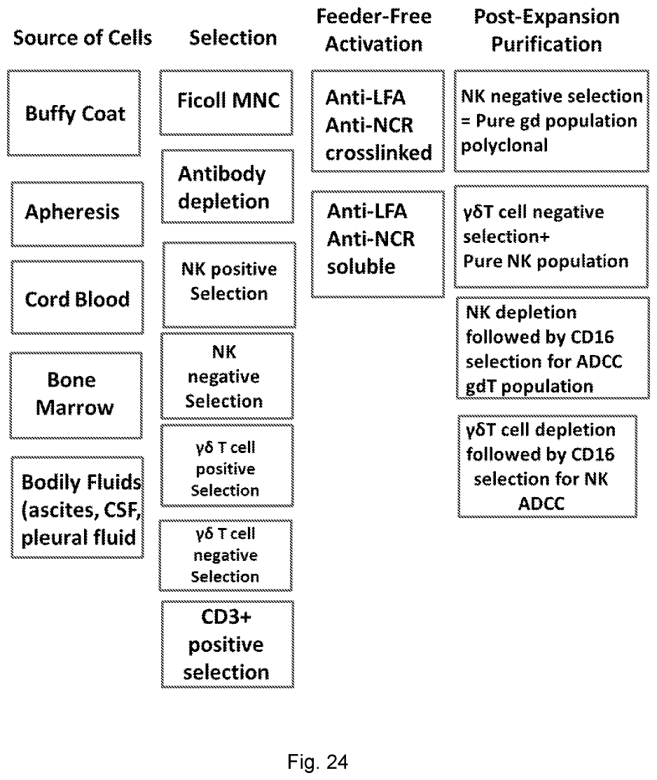

In this Example, processes for isolating a pure NK cell population and isolating a pure γδT cell population are described. A flowchart illustrating example variations of this process is presented in FIG. 24.

1. A buffy coat from a normal healthy donor undergoes a gradient cell separation with Ficoll™ performed with manual separation in a centrifuge; a Percoll gradient, a SEPAX, Cobe 2991, or elutriation system also may be used. Alternatively, an apheresis, a cord blood unit, bone marrow, or bodily fluids such as ascites, CSF or pleural fluid may be used as the source of cells with or without gradient centrifugation.

2. Mononuclear cells are used directly or after gradient separation or subjected to further cell selection such s, for example, Ficoll™ MNC, antibody depletion, NK positive selection, NK negative selection, γδT cell negative selection, and/or CD3+ positive selection. In certain instances, mononuclear cells are subjected to CD56+NK cell positive selection or negative selection using the Miltenyi magnetic bead based or other cell selection technology. Alternatively, the cells may be subjected to γδTCR+ positive selection using the Miltenyi magnetic bead based or other cell selection technology. Alternatively, the cells may be subjected to CD3+ cell depletion using the Miltenyi magnetic bead based or other cell selection technology.

3. The resulting cell population is then activated in a feeder-free culture, with BINATE medium (NK MACS™ Medium #130-107-879 Miltenyi Biotec, Inc., San Diego, Calif., USA supplemented with 5% AB serum (alternatively, other media e.g., R&D systems media, CELLGENIX media, and the like, may be used)), or other animal component free culture medium such as In Vivo or R&D Systems, or Cellgenix, with or without human serum supplementation in the presence of soluble or crosslinked anti-LFA and anti-NCR antibodies.

4. Cell expansion occurs with supplementation of cytokines such as IL-2, IL-15, IL-12, IL-18 and combinations thereof at a cell seeding concentration of 0.25-5×10⁶/ml in T75 flasks (alternatively, cell seeding concentration may range up to 1×10⁶/ml in a culture bag, flask or other large culture vessel such as a Bioreactor (G-Rex)). Media exchanges take place every 3 days (less frequently for a bioreactor (5-7 days)) with growth medium and cytokines.

5. The resulting cultures on day 7, 20, 30, 45 and 60 may comprise the BINATE population of a mix of NK and γδT cells which are then cryopreserved for administration. Alternatively, the resulting cell population may undergo a further post-expansion cell selection step, such as NK CD56+ depletion with a resulting pure γδT cell population. Alternatively, the cells may undergo positive removal or negative selection of γδT cells, with a resulting pure NK population, or positive selection of CD16, which would yield a highly enriched ADCC FcγIII receptor+ population. Alternatively, the cells may undergo NK depletion followed by CD16 selection for an ADCC γδT cell population.

### Example 25: Retroviral Constructs

In this Example, retroviral vectors CARGD2, CARCD123, and CARCD19 used in certain Examples above are described.

Vector Backbone

The constructs described below include an SFG backbone, which uses a Moloney murine leukemia virus (MoMLV)-based retroviral vector. All of env and gag-pol were removed except for the packaging sequence (psi). As a result, the vector is replication incompetent.

Producer Cell Line

The packaging cell line for producing the retrovirus constructs below was generated in the research environment at OPBG, Cell and Gene Therapy for Pediatric Tumor Laboratory using dedicated laminar flow hoods and a CO₂ incubator, and non-animal derived materials with the exception of gamma-irradiated Pharmagrade FBS (EuroClone, cat ECS0172L, Lot EUS0131906GI). The packaging cell line was generated from a cGMP-banked human based 293VEC RD114 producer received from BioVec Pharma. In particular, 293VEC RD114 cells were chosen as a packaging cell line, since they are of human origin, and allow the production of a high vector titers, suitable for large-scale clinical-grade production.

CARGD2

The iC9-CARGD2.CD28.41bb.CD3zeta (OPBG-91 vector) retroviral vector was constructed at the Ospedale Pediatrico Bambino Gesù (OPBG), “Cell and Gene Therapy for Pediatric Tumor” Laboratory. A bicistronic vector was used, which allows for simultaneous expression of two transgenes, namely inducible caspase 9 (iC9) and CARGD2 (iC9-CARGD2.CD28.41bb.CD3zeta). Single-cell cloning was performed, and the 293VEC RD114 clone (namely OPBG-91-7) that produced the highest titer (using PCR analysis for vector presence in the supernatant) was expanded, banked in Officina Farmaceutica OPBG and used for retrovirus production under cGMP conditions, after testing for sterility and mycoplasma.

In the iC9 component, the catalytic domain of Casp9 protein, expressed intracellularly, is fused to a drug-binding domain derived from the human FK506-binding protein (FKBP12) with an F36V mutation. A CAR molecule based on the single chain of the fused VH-VL region of the monoclonal antibody 14.G2A specific for the human antigen GD2, in frame with CD28 transmembrane domain and its endo domain, 4.1bb costimulatory domain and CD34 cytoplasmic domain for the transduction of the activating signal after antigen engagement, were cloned in a retroviral vector after the gene cassette including the sequence of iC9 to improve the safety aspect of the approach.

Certain functional and structural components for iC9.CARGD2.CD28.41bb.CD3z expression and activity are summarized in Table 9 below, and listed here:
5′ LTR—Retroviral long terminal repeat at 5′ end of vector (functions as promoter sequence).
ψ—Retroviral encapsidation signal (psi; necessary for packaging of RNA into virion particles).
SA—splice acceptor site.
iCasp9—the inducible caspase-9 expression cassette. iCasp9 is made up of a human FK506-binding protein (FKBP12) with an F36V mutation, connected via a 6 amino-acid Gly-Ser linker to a modified CARD domain-deleted human caspase-9.
FKBP12-F36V—an engineered FK506-binding protein containing F36V mutation to optimize binding affinity for AP1903. The FKBP12-F36V protein domain serves as the drug-binding/oligomerization domain of linked therapeutic proteins. FKBP12-F36V functions as a regulator of caspase-9. In the absence of AP1903, iCasp9 has minimal activity; AP1903 binding to FKBP12-F36V promotes dimerization and brings two caspase-9 molecules into apposition to initiate apoptosis. Thus, the FKBP12-F36V moiety functionally replaces the endogenous dimerization/activation module (Caspase Activation and Recruitment Domain; CARD) of caspase-9 that mediates Apaf-1-associated oligomerization.
Linker—synthetic Ser-Gly-Gly-Gly-Ser-Gly peptide linker used to fuse switch-regulator sequences to caspase-9.
Caspase-9—Human caspase-9 cDNA sequence (critical pro-apoptotic regulator) and therapeutic component of construct (regulated suicide gene). The endogenous dimerization/activation module (Caspase Activation and Recruitment Domain; CARD) was deleted to reduce spontaneous Apaf1-binding and hence background killing.
2A—encodes a synthetic 20 amino acid peptide from Thosea Asigna insect virus, which functions as a cleavable linker between the caspase-9 protein and CAR proteins.
Signal peptide—short amino acid sequence to allow correct translocation of the secretory proteins from the Endoplasmic Reticulum to the cellular membrane.
CAR—CAR molecule based on the single chain of the fused VH-VL region of the monoclonal antibody 14.G2A specific for the human antigen GD2, in frame with CD28 TM and costimulatory domain, 4.1bb costimulatory domain and CD3ζ cytoplasmic domain.
3′ LTR—Retroviral long terminal repeat at 3′ end of vector (functions as terminator/polyadenylation sequences).

TABLE 9

| Component | Start | End |
|---|---|---|
| LTR | 1 | 590 |
| iCasp9 cassette | 1880 | 3085 |
| FKBP12-F36V | 1880 | 2218 |
| Linker peptide | 2219 | 2236 |
| Casp9 | 2237 | 3085 |
| 2A peptide | 3086 | 3145 |
| CAR | 3146 | 4723 |
| Signal peptide | 3146 | 3208 |
| scFv | 3209 | 4000 |
| CH2-CH3 hinge | 4001 | 4055 |
| CD28TM | 4056 | 4135 |
| CD28 costimulation | 4136 | 4258 |
| 4.1bb costimulation | 4259 | 4384 |
| CD3ζ | 4385 | 4723 |
| LTR | 4896 | 5485 |
| Amp^{R} marker | 6780 | 7640 |

A reference electronic vector sequence was assembled by combining the DNA sequence files for each component of the vector construct. Since the retroviral genome is RNA-based, sequence analysis was performed on the plasmid DNA used for transfection into the 293VEC cell line (initial step in retroviral product preparation). Bi-directional sequencing was performed at Ospedale Pediatrico Bambino Gesù on the entire OPBG-91 vector. Sequencing runs were assembled using SnapGene software. No mismatched bases compared to the theoretical reference electronic sequence were identified.

CARCD123

The ΔCD19-2A-CAR-CD123-ΔCD34.CD8.41bb.CD3zeta (OPBG-242 vector) retroviral vector was constructed at the Ospedale Pediatrico Bambino Gesù (OPBG), “Cell and Gene Therapy for Pediatric Tumor” Laboratory. A bicistronic vector was used, which allows for simultaneous expression of two transgenes, namely ΔCD19 and CARCD123 (ΔCD19-2A-CAR-CD123-ΔCD34.CD8.41bb.CD3zeta). Single-cell cloning was performed, and the clone that produced the highest titer (using PCR analysis for vector presence in the supernatant) was expanded and banked as described above.

ΔCD19 represents the extracellular domain of human CD19 linked to the transmembrane portion. It has the double function to help the selection and phenotypic characterization of the genetically modified cells. A CAR molecule based on the single chain of the fused VH-VL region of the monoclonal antibody 7G3 specific for the human antigen CD123, in frame with CD8 transmembrane domain and its endo domain, 4.1bb costimulatory domain and CD34 cytoplasmic domain for the transduction of the activation signal after antigen engagement, were cloned in a retroviral vector after the gene cassette including the sequence of ΔCD19 through the use of a 2A sequence.

Certain functional and structural components for ΔCD19-2A-CAR-CD123-ΔCD34.CD8.41bb.CD3z expression and activity are summarized in Table 10 below, and listed here:
5′ LTR—Retroviral long terminal repeat at 5′ end of vector (functions as promoter sequence).
ψ—Retroviral encapsidation signal (psi; necessary for packaging of RNA into virion particles).
SA—splice acceptor site.
ΔCD19—includes the optimized human extracellular and transmembrane domains.
2A—encodes a synthetic 20 amino acid peptide from Thosea Asigna insect virus.
Signal peptide—short amino acid sequence to allow the correct translocation of the secretory proteins from the Endoplasmic Reticulum to the cellular membrane.
ΔCD34—includes a short peptide derived from human CD34, helping to detect CAR+ T cells after transduction.
CAR—CAR molecule based on the single chain of the fused VH-VL region of the monoclonal antibody 7G3 specific for the human antigen CD123, in frame with CD8 TM and costimulatory domain, 4.1bb costimulatory domain and CD3 cytoplasmic domain.
3′ LTR—Retroviral long terminal repeat at 3′ end of vector (functions as terminator/polyadenylation sequences).

TABLE 10

| Component | Start | End |
|---|---|---|
| LTR | 397 | 990 |
| dCD19 | 2282 | 3280 |
| SvFv 7G3 | 3404 | 3745 |
| 2A peptide | 3746 | 3769 |
| SvFv 7G3 | 3770 | 4123 |
| ΔCD34 | 4124 | 4183 |
| CD8 stalk | 4184 | 4309 |
| CD8TM | 4310 | 4372 |
| CD8 Cyt | 4373 | 4420 |
| 4.1bb costimulation | 4427 | 4552 |
| CD3ζ | 4553 | 4891 |
| 3′ LTR | 5069 | 5635 |
| Amp^{R} promoter | 6848 | 6952 |
| Amp^{R} | 6953 | 7813 |

A reference electronic vector sequence was assembled by combining the DNA sequence files for each component of the vector construct. Since the retroviral genome is RNA-based, sequence analysis was performed on the plasmid DNA used for transfection into the 293VEC cell line (initial step in retroviral product preparation). Bi-directional sequencing was performed at Ospedale Pediatrico Bambino Gesù on the entire OPBG-242 vector. Sequencing runs were assembled using SnapGene software. No mismatched bases compared to the theoretical reference electronic sequence were identified.

CARCD19

The therapeutic retroviral construct SFG-iC9-Car.CD19.41bb encodes a synthetic ligand-inducible human caspase-9 cDNA linked to the single chain of the fused VH-VL region of the monoclonal antibody specific for murine antigen CD19, 4.1bb costimulatory domain. The assembly is the same as for CARGD2 described above except it is 2nd generation. The retroviral vector was constructed at an EU Cell and Gene Therapy Laboratory. Single-cell cloning was performed, and the clone that produced the highest titer (using PCR analysis for vector presence in the supernatant) was expanded, banked in the cGMP facility and used for retrovirus production under cGMP conditions, after testing for sterility and mycoplasma.

Certain functional and structural components for SFG-iC9-Car.CD19.41bb expression and activity are listed here:
5′ LTR—Retroviral long terminal repeat at 5′ end of vector (functions as promoter sequence).
ψ—Retroviral encapsidation signal (psi; necessary for packaging of RNA into virion particles).
SA—splice acceptor site.
iCasp9—the inducible caspase-9 expression cassette. iCasp9 is made up of a human FK506-binding protein (FKBP12) with an F36V mutation, connected via a 6 amino-acid Gly-Ser linker to a modified CARD domain-deleted human caspase-9.
FKBP12-F36V—an engineered FK506-binding protein containing F36V mutation to optimize binding affinity for AP1903. The FKBP12-F36V protein domain serves as the drug-binding/oligomerization domain of linked therapeutic proteins. FKBP12-F36V functions as a regulator of caspase-9. In the absence of AP1903, iCasp9 has minimal activity; AP1903 binding to FKBP12-F36V promotes dimerization and brings two caspase-9 molecules into apposition to initiate apoptosis. Thus, the FKBP12-F36V moiety functionally replaces the endogenous dimerization/activation module (Caspase Activation and Recruitment Domain; CARD) of caspase-9 that mediates Apaf-1-associated oligomerization.
Linker—synthetic Ser-Gly-Gly-Gly-Ser-Gly peptide linker used to fuse switch-regulator sequences to caspase-9.
Caspase-9—Human caspase-9 cDNA sequence (critical pro-apoptotic regulator) and therapeutic component of construct (regulated suicide gene). The endogenous dimerization/activation module (Caspase Activation and Recruitment Domain; CARD) was deleted to reduce spontaneous Apaf1-binding and hence background killing.
2A—encodes a synthetic 20 amino acid peptide from Thosea Asigna insect virus, which functions as a cleavable linker between the caspase-9 protein and CAR proteins.
Signal peptide—short amino acid sequence to allow correct translocation of the secretory proteins from the Endoplasmic Reticulum to the cellular membrane.
CAR—CAR molecule based on the single chain of the fused VH-VL region of the monoclonal antibody specific for murine antigen CD19, 4.1bb costimulatory domain.
3′ LTR—Retroviral long terminal repeat at 3′ end of vector (functions as terminator/polyadenylation sequences).

### Example 26: Examples of Certain Non-Limiting Embodiments

Listed hereafter are non-limiting examples of certain embodiments of the technology.

A1. A method for manufacturing a composition comprising a population of cells enriched in NK cells and gamma.delta T cells, comprising:
obtaining a sample containing cells from one or more subjects;
depleting alpha.beta T cells from the sample under conditions that generate a depleted cell population comprising NK cells and gamma.delta T cells; and
exposing the depleted cell population to activation conditions comprising contacting the depleted cell population with: (a) at least one exogenous polypeptide that immunospecifically binds to a cell adhesion polypeptide, and (b) at least one exogenous polypeptide that immunospecifically binds to a different polypeptide than the cell adhesion polypeptide and is expressed on the surface of one or more cells of the sample population; and
exposing the depleted cell population to expansion conditions comprising contacting the depleted cell population with at least one supplemental polypeptide, thereby generating a composition comprising a population of cells enriched in NK cells and gamma.delta T cells.

A1.1. A method for manufacturing a composition comprising a population of cells enriched in NK cells and gamma.delta T cells, comprising:
obtaining a sample containing cells from one or more subjects;
exposing the sample to activation conditions comprising contacting the sample with: (a) at least one exogenous polypeptide that immunospecifically binds to a cell adhesion polypeptide, and (b) at least one exogenous polypeptide that immunospecifically binds to a different polypeptide than the cell adhesion polypeptide and is expressed on the surface of one or more cells of the sample population, wherein (a) or (b) is soluble or (a) and (b) are soluble; and
exposing the sample to expansion conditions comprising contacting the sample with at least one supplemental polypeptide, thereby generating a composition comprising a population of cells enriched in NK cells and gamma.delta T cells.

A2. The method of embodiment A1 or A1.1, wherein the at least one supplemental polypeptide is selected such that the amount of NK cells relative to the amount of gamma.delta T cells in the population is dependent on the amount and/or type of the at least one supplemental polypeptide.

A2.1. The method of embodiment A1, A1.1 or A2, wherein the at least one supplemental polypeptide increases or decreases the amount of NK cells relative to gamma.delta T cells in the population of cells after the depleted cell population is contacted with the at least one supplemental polypeptide.

A3. The method of any of embodiments A1, A1.1, A2 or A2.1, wherein the activation conditions are free of serum from a non-human animal.

A4. The method of any of embodiments A1 to A3, wherein the expansion conditions are free of serum from a non-human animal.

A5. The method of any of embodiments A1 to A4, wherein the activation conditions are free of feeder cells.

A6. The method of any of embodiments A1 to A5, wherein the expansion conditions are free of feeder cells.

A7. The method of any of embodiments A1 to A6, wherein the sample is chosen from among peripheral blood, liver tissue, epithelial tissue, bone marrow and cord blood.

A8. The method of embodiment A7, wherein the sample is peripheral blood.

A9. The method of embodiment A8, wherein the peripheral blood sample is a processed sample.

A10. The method of embodiment A7, wherein the sample is cord blood.

A11. The method of embodiment A10, wherein the cord blood sample is a processed sample.

A12. The method of any of embodiments A1 to A11, wherein the exogenous polypeptide in (b) immunospecifically binds to a NK cell activation receptor, a gamma.delta T cell activation receptor, or both.

A13. The method of embodiment A12, wherein the receptor is NKp30, NKp44 or NKp46.

A14. The method of embodiment A13, wherein the receptor is NKp46.

A15. The method of any of embodiments A1 to A14, wherein the exogenous polypeptide in (a) immunospecifically binds to CD2.

A16. The method of any of embodiments A1 to A15, wherein the exogenous polypeptide in (a) or (b), or (a) and (b), is an antibody or an antigen-binding fragment thereof.

A17. The method of any of embodiments A1 to A16, wherein at least one of (a) or (b) is soluble.

A17.1. The method of any of embodiments A1 to A17, wherein the exogenous polypeptides in (a) and (b) are both soluble.

A18. The method of any of embodiments A1 to A16, wherein the exogenous polypeptide in (a) or the exogenous polypeptide in (b) is bound to a substrate.

A19. The method of any of embodiments A1 to A18, wherein the activation conditions comprise contacting the sample or depleted cell population with at least two exogenous polypeptides.

A20. The method of embodiment A19, wherein the first exogenous polypeptide immunospecifically binds to CD2 and the second exogenous polypeptide immunospecifically binds to NKp46.

A21. The method of embodiment A19 or A20, wherein the first exogenous polypeptide and/or the second exogenous polypeptide is/are an antibody or an antigen-binding fragment thereof.

A22. The method of any of embodiments A1 to A21, wherein the polypeptide components of the activation conditions consist essentially of, or consist of:
(a) an exogenous polypeptide that immunospecifically binds to the cell adhesion polypeptide CD2; and
(b) an exogenous polypeptide that is different than the exogenous polypeptide in (a) and immunospecifically binds to NKp46.

A23. The method of any of embodiments A1 to A22, wherein the supplemental polypeptide is a cytokine and/or a polypeptide that immunospecifically binds to a receptor on a gamma.delta T cell.

A24. The method of embodiment A23, wherein the expansion conditions comprise contacting the sample or depleted cell population with at least one supplemental polypeptide that is a cytokine and, optionally, a supplemental polypeptide that immunospecifically binds to a receptor on a gamma.delta T cell.

A25. The method of embodiment A24, wherein the cytokine is an interleukin (IL).

A26. The method of any of embodiments A23 to A25, wherein the at least one supplemental polypeptide comprises IL-2, IL-4, IL-15 or any combination thereof.

A27. The method of any of embodiments A23 to A26, wherein the expansion conditions comprise contacting the sample or depleted cell population with:
(a) an IL-2 polypeptide and, optionally, a polypeptide that immunospecifically binds to a receptor on a gamma.delta T cell;
(b) an IL-15 polypeptide; or
(c) an IL-2 polypeptide and an IL-15 polypeptide and, optionally, a polypeptide that immunospecifically binds to a receptor on a gamma.delta T cell.

A28. The method of any of embodiments A23 to A27, wherein the receptor on the gamma.delta T cell is CD3.

A29. The method of any of embodiments A23 to A28, wherein the polypeptide that immunospecifically binds to a receptor on a gamma.delta T cell is an antibody or antigen-binding fragment thereof.

A30. The method of embodiment A29, wherein the expansion conditions comprise contacting the sample with:
(a) an IL-2 polypeptide;
(b) an IL-15 polypeptide;
(c) an IL-2 polypeptide and an IL-15 polypeptide;
(d) an IL-2 polypeptide and an antibody that immunospecifically binds CD3; or
(e) an IL-2 polypeptide, an IL-15 polypeptide and an antibody that immunospecifically binds CD3.

A31. The method of embodiment A30, wherein the antibody that immunospecifically binds CD3 is OKT3.

A32. The method of any of embodiments A1 to A31, wherein the activation and expansion conditions are performed simultaneously or sequentially in any order.

A33. The method of any of embodiments A1 to A32, wherein:
the at least one exogenous polypeptide also can function as a supplemental polypeptide; or
the at least one supplemental polypeptide also can function as an exogenous polypeptide; or
the at least one exogenous polypeptide also can function as a supplemental polypeptide and the at least one supplemental polypeptide also can function as an exogenous polypeptide.

A34. The method of any of embodiments A30 to A33, wherein:
(i) the expansion conditions comprise contacting the sample or depleted cell population with an IL-2 polypeptide; and
(ii) the resulting population of cells enriched in NK cells and gamma.delta T cells comprises about 25-30% NK cells and about 70-75% gamma.delta T cells.

A35. The method of any of embodiments A30 to A33, wherein:
(i) the expansion conditions comprise contacting the sample or depleted cell population with an IL-15 polypeptide; and
(ii) the resulting population of cells enriched in NK cells and gamma.delta T cells comprises about 80-99% NK cells and about 1-20% gamma.delta T cells.

A36. The method of any of embodiments A30 to A33, wherein:
(i) the expansion conditions comprise contacting the sample or depleted cell population with an IL-2 polypeptide and an antibody that immunospecifically binds CD3; and
(ii) the resulting population of cells enriched in NK cells and gamma.delta T cells comprises about 40-45% NK cells and about 55-60% gamma.delta T cells.

A37. The method of any of embodiments A1 to A36, wherein the expansion conditions comprise:
contacting the sample or depleted cell population with a first set of conditions comprising one or more supplemental polypeptides, resulting in a first cell population comprising a first ratio of NK cells to gamma.delta T cells; and
contacting the first cell population with a second set of conditions comprising one or more supplemental polypeptides, resulting in a second cell population comprising a desired final ratio of NK cells to gamma.delta T cells, wherein the first set of conditions is different than the second set of conditions.

A38. The method of embodiment A37, wherein the first cell population is washed prior to contact with the second set of conditions.

A39. The method of embodiment A37 or A38, wherein:
the first set of conditions comprise IL-2 and the second set of conditions comprise IL-15;
the first set of conditions comprise IL-15 and the second set of conditions comprise IL-2;
the first set of conditions comprise IL-2 and an antibody that immunospecifically binds CD3 and the second set of conditions comprise IL-15; or
the first set of conditions comprise IL-15 and an antibody that immunospecifically binds CD3 and the second set of conditions comprise IL-2 and an antibody that immunospecifically binds CD3.

A40. The method of embodiment A39, wherein the antibody that immunospecifically binds CD3 is OKT3.

A41. The method of any of embodiments A1.1 and A2 to A40, further comprising:
prior to exposing the sample to the activation and expansion conditions, depleting alpha.beta T cells from the sample, thereby generating a depleted cell population; and
subjecting the depleted cell population to the activation and expansion conditions, whereby a composition comprising a population of cells enriched in NK cells and gamma.delta T cells is obtained.

A41.1. The method of any of embodiments A1 to A41, wherein, prior to activation and expansion, the sample or the depleted cell population are not exposed to conditions that select for NK cells or gamma.delta T cells, or deplete cells other than the alpha-beta T cells.

A41.2. The method of embodiment A41.1, wherein, prior to activation and expansion, the sample or the depleted cell population are not exposed to conditions that deplete CD3+ cells.

A42. The method of any of embodiments A1 to A41.1, wherein the cells of the sample or depleted cell population do not comprise exogenous nucleic acid before, during or after activation and expansion.

A43. The method of any of embodiments A1 to A41.1, wherein the cells of the sample or depleted cell population do not comprise exogenous nucleic acid encoding a tumor necrosis factor receptor, a chimeric antigen receptor (CAR), a myeloid differentiation primary response protein or an innate immune signal transduction adaptor before, during or after activation and expansion.

A44. The method of any of embodiments A1 to A43, wherein the cells of the sample or depleted cell population are not genetically modified before, during or after activation and expansion.

A45. The method of any of embodiments A1 to A44, further comprising, subjecting the population of cells enriched in NK cells and gamma.delta T cells to a treatment whereby the gamma.delta cells are depleted and the resulting population consists essentially of, or consists of, NK cells.

A46. The method of any of embodiments A1 to A44, further comprising, subjecting the population of cells enriched in NK cells and gamma.delta T cells to a treatment whereby the NK cells are depleted and the resulting population consists essentially of, or consists of, gamma.delta T cells.

A47. The method of any of embodiments A1 to A45, further comprising, subjecting the population of cells enriched in NK cells and gamma.delta T cells to a positive selection for NK cells whereby a cell population that consists essentially of, or consists of, NK cells is obtained.

A48. The method of any of embodiments A1 to A44 and A46, further comprising, subjecting the population of cells enriched in NK cells and gamma.delta T cells to a positive selection for gamma.delta cells whereby a cell population that consists essentially of, or consists of, gamma.delta cells is obtained.

A49. The method of any of embodiments A1 to A48, wherein the expansion conditions comprise incubation of the sample or depleted cell population in a feeder cell free medium for between about one week to about 10 weeks, whereby a composition comprising an expanded population of cells enriched in NK cells and gamma.delta T cells is obtained.

A50. The method of embodiment A49, wherein the culture conditions comprise incubation of the sample or depleted cell population in a feeder cell free medium for about 7, 8, 9, 10, 11, 12, 13, 14, 15, 16, 17, 18, 19, 20, 21, 25, 30, 35, 40, 45, 50, 55 or 60 or more days or about 2, 3, 4, 5, 6, 7, 8, 9 or 10 weeks.

A51. The method of any of embodiments A1 to A50, wherein the population of cells enriched in NK cells and gamma.delta T cells is expanded by greater than about 2 logs in 30 days under the expansion conditions.

A52. The method of embodiment A51, wherein the population of cells is expanded by greater than about 3 logs in 30 days under the expansion conditions.

A53. The method of any of embodiments A1 to A52, wherein the expanded population of cells enriched in NK cells and gamma.delta T cells is free of exhausted cells.

A54. The method of any of embodiments A1 to A53, wherein the expanded population of cells enriched in NK cells and gamma.delta T cells is free of exhausted cells after 60 days of the expansion conditions.

A55. The method of any of embodiments A1 to A54, wherein less than 5%, 4%, 3% or 2% of the NK cells in the expanded population of cells enriched in NK cells and gamma.delta T cells comprise a PD-1 marker and/or about 20%, 15%, 10% or less of the total cells in the expanded population or the gamma.delta T cells in the expanded population comprise the PD-1 marker.

A56. The method of any of embodiments A1 to A55, wherein the population of cells enriched in NK cells and gamma.delta T cells comprises one or more of the following activation markers as a percentage of the total number of cells in the population:
(a) 90% or greater KIR5;
(b) 10% or greater SIGLEC-7;
(c) 60% or greater KIR3D51;
(d) 10% or greater KIR2DL1;
(e) 25% or greater NKp30, NKp44 or NKp46;
(f) 35% or more NKG2D;
(g) 90% or more DNAM1;
(h) 85% or more NTBA;
(i) 95% or greater CD2; and
(j) 55% or greater KIR3DS1.

A57. The method of any of embodiments A1 to A56, wherein the population of cells enriched in NK cells and gamma.delta T cells comprises 80% or more innate immune cells.

A58. The method of embodiment A57, wherein between about 70% to about 100%, or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the cells are CD56+.

A59. The method of embodiment A57 or A58, wherein between about 10% to about 40%, or at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the cells are CD16+.

A60. The method of any of embodiments A57 to A59, wherein less than 10%, less than 5%, less than 4%, less than 3% or less than 2% of the cells are CD57+.

A61. The method of any of embodiments A1 to A60, wherein the sample or depleted cell population does not comprise CD4+CD8+ cells during or after activation and expansion.

A62. The method of any of embodiments A1 to A61, wherein the activation conditions and the expansion conditions do not comprise a bisphosphonate.

A63. The method of embodiment A62, wherein the bisphosphonate is pamidronate or zoledronate.

A64. The method of any of embodiments A1 to A63, wherein the gamma.delta T cells are polyclonal with respect to V.delta.1 and V.delta.2 expression.

A65. The method of embodiment A64, wherein the polyclonal gamma.delta T cells comprise at least one subpopulation selected from among V.delta.1+ and V.delta.1- and at least one subpopulation selected from among V.delta.2+ and V.delta.2-.

A66. A composition obtainable by, or obtained by, the method of any of embodiments A1 to A65.

B1. A composition comprising a modified population of peripheral blood cells, wherein the population comprises:
a plurality of NK cells and a plurality of gamma.delta T cells;
is alpha.beta T cell depleted; and
is free of feeder cells.

B2. The composition of embodiment B1, wherein:
between about 25% to about 45% of the cells are NK cells and between about 55% to about 75% of the cells are gamma.delta T cells;
between about 25% to about 30% of the cells are NK cells and between about 70% to about 75% of the cells are gamma.delta T cells;
between about 80% to about 99% of the cells are NK cells and between about 1% to about 20% of the cells are gamma.delta T cells; or
between about 40% to about 45% of the cells are NK cells and between about 55% to about 60% of the cells are gamma.delta T cells.

B3. The composition of embodiment B1 or B2, wherein 30% or more of the cells are activated.

B4. The composition of any of embodiments B1 to B3, wherein the modified population of cells comprises one or more of the following activation markers as a percentage of the total number of cells in the population:
(a) 90% or greater KIR5;
(b) 10% or greater SIGLEC-7;
(c) 60% or greater KIR3D51;
(d) 10% or greater KIR2DL1;
(e) 25% or greater NKp30, NKp44 or NKp46;
(f) 35% or more NKG2D;
(g) 90% or more DNAM1;
(h) 85% or more NTBA;
(i) 95% or greater CD2; and
(j) 55% or greater KIR3DS1

B5. The composition of any of embodiments B1 to B4, wherein the modified population comprises 80% or more innate immune cells.

B6. The composition of any of embodiments B1 to B5, wherein the modified population is enriched in activated cytotoxic cells that are CD56+.

B7. The composition of any of embodiments B1 to B6, wherein the modified population is enriched in activated cytotoxic cells that are CD57-.

B8. The composition of any of embodiments B1 to B7, wherein the population is enriched in activated cytotoxic cells that are CD56+CD57-.

B9. The composition of any of embodiments B6 to B8, wherein between about 80% to about 100%, or at least about 80%, 81%, 82%, 83%, 84%, 85%, 86%, 87%, 88%, 89%, 90%, 91%, 92%, 93%, 94%, 95%, 96%, 97%, 98%, 99% or 100% of the cells are CD56+.

B10. The composition of any of embodiments B6 to B9, wherein between about 10% to about 40%, or at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the cells are CD16+.

B11. The composition of any of embodiments B6 to B10, wherein less than 5%, less than 4%, less than 3% or less than 2% of the cells are CD57+.

B12. The composition of any of embodiments B1 to B11 that is substantially free of cells other than NK cells and gamma.delta T cells.

B13. The composition of any of embodiments B1 to B12 that comprises less than five percent NKT cells.

B14. The composition of any of embodiments B1 to B13 that comprises less than one percent NKT cells.

B15. The composition of any of embodiments B1 to B14 that comprises less than 0.1 percent NKT cells.

B16. The composition of any of embodiments B1 to B15 that comprises less than two percent alpha.beta T cells.

B17. The composition of any of embodiments B1 to B16 that comprises less than one percent alpha.beta T cells.

B18. The composition of any of embodiments B1 to B17 that comprises less than 0.1 percent alpha.beta T cells.

B19. The composition of any of embodiments B1 to B18, wherein a subset of NK cells in the population are CD16+ cells.

B20. The composition of any of embodiments B1 to B19, wherein the majority of gamma.delta T cells are CD57− cells.

B21. The composition of any of embodiments B1 to B20, wherein the majority of NK cells are CD57− cells.

B22. The composition of any of embodiments B1 to B21, wherein the gamma.delta T cells are polyclonal with respect to V.delta.1 and V.delta.2 expression.

B23. The composition of embodiment B22, wherein the polyclonal gamma.delta T cells comprise at least one subpopulation selected from among V.delta.1+ and V.delta.1- and at least one subpopulation selected from among V.delta.2+ and V.delta.2-.

B24. The composition of any of embodiments B1 to B23, wherein:
a majority of gamma.delta T cells express V.delta.1 and a minority of gamma.delta T cells express V.delta.2; or
a minority of gamma.delta T cells express V.delta.1 and a majority of gamma.delta T cells express V.delta.2 expression.

B25. The composition of any of embodiments B1 to B24, wherein:
a minority of cells in the population are CD3 positive cells and a majority of cells in the population are CD3 negative cells; or
a majority of cells in the population are CD3 positive cells and a minority of cells in the population are CD3 negative cells.

B26. The composition of any of embodiments B1 to B25, wherein the ratio of NK cells to gamma.delta T cells is greater than 1.

B27. The composition of any of embodiments B1 to B25, wherein the ratio of NK cells to gamma.delta T cells is less than 1.

B28. The composition of embodiment B26, wherein the modified population of cells comprises about 98-99% NK cells and about 1-2% gamma.delta T cells.

B29. The composition of embodiment B27, wherein the modified population of cells comprises between about 25% to about 45% NK cells and between about 55% to about 75% gamma.delta T cells.

B30. The composition of embodiment B27, wherein the modified population of cells comprises about 25-30% NK cells and about 70-75% gamma.delta T cells.

B31. The composition of embodiment B27, wherein the modified population of cells comprises about 40-45% NK cells and about 55-60% gamma.delta T cells.

B32. The composition of any of embodiments B1 to B31, wherein between about 50% to about 99% or more, or greater than or equal to about 50%, 51%, 52%, 53%, 54%, 55%, 56%, 57%, 58%, 59%, 60%, 6%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99% 80%, 81%, 82%, 83%, 84%, 85%, 86%, 87%, 88%, 89%, 90%, 91%, 92%, 93%, 94%, 95%, 96%, 97%, 98%, 99% or fractions thereof, up to 100%, of the NK cells and/or the gamma.delta T cells are CD8+.

B33. The composition of embodiment B32, wherein less than 2% of the NK cells and/or the gamma.delta T cells are CD4+.

B34. The composition of embodiment B32 or B33, wherein less than 2% of the NK cells and/or the gamma.delta T cells are CD8+CD4+.

B35. The composition of any of embodiments B32 to B34, wherein a fraction of between about 15% to about 30% of the NK cells and/or between about 55% to 85% the gamma.delta T cells are CD8-CD4-.

B36. The composition of any of embodiments B1 to B35, wherein between about 30% to about 99% or more, or at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or fractions thereof, up to 100% of the cells in the population further comprise a genetic modification comprising an exogenous polynucleotide, a mutated polynucleotide, a deleted polynucleotide or combinations thereof.

B36.1 The composition of embodiment B36, comprising a plurality of NK cells and a plurality of gamma.delta T cells.

B37. The composition of embodiment B36 or B36.1, wherein at least about 95%, 96%, 97%, 98%, 99% of the cells in the population comprise the genetic modification, or about 100% or 100% of the cells in the population comprise the genetic modification.

B38. The composition of any of embodiments B36, B36.1 or B37, wherein the genetic modification comprises an exogenous polynucleotide.

B39. The composition of embodiment B38, wherein the exogenous polynucleotide is in a retroviral vector or a lentiviral vector.

B40. The composition of embodiment B38, wherein the exogenous polynucleotide is integrated into genomes of one or more cells of the modified cell population.

B41. The composition of any of embodiments B36 to B40, wherein the cells in the population comprise a chimeric antigen receptor (CAR).

B42. The composition of embodiment B41, wherein the chimeric antigen receptor comprises a binding molecule portion that immunospecifically binds to one or more of CD19, GD2, HER3, B7H3, CD123 or CD30.

B43. The composition of any of embodiments B1 to B42, wherein the population comprising the plurality of NK cells and the plurality of gamma.delta T cells is derived from the peripheral blood cells of more than one subject.

C1. A pharmaceutical composition comprising the composition of any one of embodiments B1 to B43 and a pharmaceutically acceptable carrier.

D1. A method of making a modified immune cell, comprising one or more of
(a) adding an exogenous polynucleotide to the composition of any of embodiments A66 and B1 to B35.
(b) mutating a polynucleotide in one or more cells of the composition of any of embodiments A66 and B1 to B35; or
(c) deleting a polynucleotide in one or more cells of the composition of any of embodiments A66 and B1 to B35.

D2. The method of embodiment D1, wherein the genetic modification is by retroviral transduction, lentiviral transduction, electroporation, transfection, CRISPR/cas9 or TALENS.

D3. The method of embodiment D1 or D2, wherein the genetic modification consists, or consists essentially of, adding an exogenous polynucleotide as in (a).

D4. The method of any of embodiments D1-D3, wherein the genetic modification comprises adding an exogenous polynucleotide as in (a) and/or or mutating a polynucleotide as in (b) and the exogenous polynucleotide and/or the mutated polynucleotide is integrated into the genome of the immune cell.

D5. The method of embodiment D4, wherein the integration is by electroporation, transfection, CRISPR/cas9 or TALENS.

D6. The method of any of embodiments D1 to D5, wherein the exogenous polynucleotide encodes a chimeric antigen receptor (CAR).

D7. The method of embodiment D6, wherein the chimeric antigen receptor comprises a binding molecule portion that immunospecifically binds to one or more of CD19, GD2, HER3, B7H3, CD123 or CD30.

D8. The method of any of embodiments D1 to D7, wherein between about 30% to about 99% or more, or at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% 91%, 92%, 93%, 94%, 95%, 96%, 97%, 98%, 99% or fractions thereof, up to 100% of the cells in the population comprise the genetic modification.

D8.1 The method of embodiment D8, wherein the cells in the population comprise a plurality of NK cells and a plurality of gamma.delta T cells.

D9. The method of embodiment D8 or D8.1, wherein the genetic modification comprises an exogenous polynucleotide.

D10. The method of embodiment D9, wherein about 100% or 100% of the cells in the population comprise the exogenous polynucleotide.

E1. A kit, comprising the composition of any one of embodiments A66 and B1 to B43, or the pharmaceutical composition of embodiment C1, optionally, instructions for use and, optionally, a cytokine.

E2. The kit of embodiment E1, wherein the composition or the pharmaceutical composition are at negative 4 degrees Celsius or less.

E3. The kit of embodiment E2, wherein the composition or the pharmaceutical composition are at about negative 75 degrees Celsius to about negative 80 degrees Celsius.

E4. The kit of any of embodiments E1 to E3, comprising about 1×10⁵ cells to about 1×10¹² cells.

E5. The kit of any of embodiments E1 to E4, wherein the cytokine is an interleukin polypeptide.

E6. The kit of embodiment E5, wherein the interleukin peptide is IL-2, IL-4 or IL-15.

E7. The kit of any of embodiments E1 to E6 that is free of non-human serum and/or is free of bovine serum.

E8. The kit of any of embodiments E1 to E7 that is free of xenogen.

E9. The kit of any one of embodiments E1 to E8 that is free of exogenous feeder cells.

E10. The kit of any one of embodiments E1 to E9 in a unit dosage form.

E11. The kit of embodiment E10, wherein the unit dosage form is from about 1×10⁶ cells to about 1×10¹² cells.

F1. A collection of cells from different donor subjects, comprising a plurality of containers each comprising cells from one or more donor subjects, wherein each container comprises the composition of any one of embodiments A66 and B1 to B43, the pharmaceutical composition of embodiment C1, or the kit of any of embodiments E1 to E11.

G1. A method for treating a cancer or an infection, comprising administering to a subject in need thereof the composition of any one of embodiments A66 and B1 to B43, the pharmaceutical composition of embodiment C1, or the kit of any of embodiments E1 to E11 in an amount effective to treat the cancer or infection, wherein cells in the composition, the pharmaceutical composition or kit are allogeneic with respect to the subject.

G2. A method for treating a cancer or an infection, comprising administering to a subject in need thereof the composition of any one of embodiments A66 and B1 to B43, the pharmaceutical composition of embodiment C1, or the kit of any of embodiments E1 to E11 in an amount effective to treat the cancer or infection, wherein cells in the composition, the pharmaceutical composition or kit are autologous with respect to the subject.

G3. The method of embodiment G1 or embodiment G2, comprising administering the composition on two or more separate days to the subject.

G4. The method of embodiment G2 or embodiment G3, wherein the donor of the cells is the recipient of the treatment.

G5. The method of embodiment G1 or embodiment G3, wherein the donor of the cells is not the recipient of the treatment.

G6. The method of embodiment G5, wherein the recipient of the treatment is susceptible to GvHD if treated with alpha.beta T cells from the donor.

G7. The method of any of embodiments G1 to G6, wherein the treatment is administered at between about 1 unit dosage to about 36 or more unit dosages at intervals of between about 2 weeks to about 4 weeks.

G8. The method of any of embodiments G1 to G6, wherein the treatment is administered as a single unit dosage one two, three, four or up to five times daily, or one, two, three, four, five, six, seven, eight, nine or ten or more times over the course of several days, weeks or months, or every other day, or one, two, three four, five or six times a week.

G9. The method of any of embodiments G1 to G8, wherein the treatment is administered intravenously (IV), intrathecally or intramuscularly (IM), intraperitoneally (IP), intra-pleurally, into the joint space or is injected or implanted at or near the site of the cancer or infection.

G10. The method of embodiment G8 or G9, wherein the unit dosage comprises between about 104 to about 10¹⁰ cells per kilogram of weight of the subject, or between about 10⁶ to about 10¹² cells per subject.

G11. The method of embodiment G10, wherein the unit dosage is about 10¹⁰ cells per subject, or about 10⁸ cells per kilogram of weight of the subject.

G12. The method of any of embodiments G1 to G11, wherein the treatment is for cancer.

G13. The method of embodiment G12, wherein the cancer is selected from among a lung cancer, melanoma, breast cancer, prostate cancer, colon cancer, renal cell carcinoma, ovarian cancer, neuroblastoma, rhabdomyosarcoma, leukemia or lymphoma, Hodgkin's lymphoma or childhood acute lymphoblastic leukemia, non-Hodgkin lymphoma, a mastocytoma or a mast cell tumor, an ovarian cancer or carcinoma, pancreatic cancer, a non-small cell lung cancer, small cell lung cancer, hepatocarcinoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a colon carcinoma, cervical carcinoma, sarcoma, bladder tumor, tumor of the reticuloendothelial tissues, Wilm's tumor, a bone cancer, an osteosarcoma, a renal cancer, or head and neck cancer, oral cancer, a laryngeal cancer, metastatic disease or an oropharyngeal cancer.

G14. The method of embodiment G12 or G13, wherein a second agent is co-administered with the composition, pharmaceutical composition or kit.

G15. The method of embodiment G14, wherein the second agent is an antibody that immunospecifically binds to a cancer-associated antigen.

G16. The method of embodiment G15, wherein the cancer-associated antigen is selected from the group consisting of α-fetoprotein (AFP), σ-actinin-4, A3, antigen specific for A33 antibody, ART-4, B7, B7-H3, Ba 733, BAGE, BrE3-antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CDC27, CDK-4/m, CDKN2A, CTLA4, CXCR4, CXCR7, CXCL12, HIF-1α, colon-specific antigen-p (CSAp), CEA (CEACAM-5), CEACAM-6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-s, HLA-DR, HM1.24, human chorionic gonadotropin (HCG) and subunits thereof, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), GD2, MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD1 receptor, placental growth factor, p53, PLAGL2, prostatic acid phosphatase, PSA, PRAME, PSMA, PIGF, ILGF, ILGF-R, L-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, and Kras.

G17. The method of embodiment G15 or G16, wherein the antibody is selected from among hR1 (anti-IGF-1R), hPAM4 (anti-mucin), KC4 (anti-mucin), hA20 (anti-CD20), hA19 (anti-CD19), hIMMU31 (anti-AFP), hLL1 (anti-CD74), hLL2 (anti-CD22), anti-CD19/CD22 bispecific antibody, RFB4 (anti-CD22), hMu-9 (anti-CSAp), hL243 (anti-HLA-DR), hMN-14 (anti-CEACAM-5), hMN-15 (anti-CEACAM-6), hRS7 (anti-TROP-2), hMN-3 (anti-CEACAM-6), CC49 (anti-TAG-72), J591 (anti-PSMA), D2/B (anti-PSMA), G250 (anti-carbonic anhydrase IX), dinutuximab (anti-GD2), infliximab (anti-TNF-α), certolizumab pegol (anti-TNF-α), adalimumab (anti-TNF-α), alemtuzumab (anti-CD52), bevacizumab (anti-VEGF), cetuximab (anti-EGFR), gemtuzumab (anti-CD33), ibritumomab tiuxetan (anti-CD20), panitumumab (anti-EGFR), rituximab (anti-CD20), tositumomab (anti-CD20), GA101 (anti-CD20), trastuzumab (anti-HER2/neu), tocilizumab (anti-IL-6 receptor), basiliximab (anti-CD25), daclizumab (anti-CD25), efalizumab (anti-CD11a), muromonab-CD3 (anti-CD3 receptor), natalizumab (anti-α4 integrin), BWA-3 (anti-histone H2A/H4), LG2-1 (anti-histone H3), MRA12 (anti-histone H1), PR1-1 (anti-histone H2B), LG11-2 (anti-histone H2B), and LG2-2 (anti-histone H2B).

G18. The method of any of embodiments G1 to G11, wherein the treatment is for an infection.

G19. The method of embodiment G18, wherein the infection is characterized by the presence of a bacterial, fungal, viral or protozoan pathogen).

G20. The method of embodiment G19, wherein the infection is selected from the group consisting of Herpes, ebola, West Nile virus, Vaccinia virus, Epstein Barr virus, Hepatitis A Virus (HAV); Hepatitis B Virus (HBV); Hepatitis C Virus (HCV); herpes viruses (e.g. HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococca, Rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidoides immitis, Histoplasma capsulatum, Leptospirosis, Borrelia burgdorferi, helminth parasite (hookworm, tapeworms, flukes, flatworms (e.g. Schistosomia), Giardia lambia, trichinella, Dientamoeba Fragilis, Trypanosoma brucei, Trypanosoma cruzi, or Leishmania donovani.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for manufacturing a composition comprising about 80-99% NK cells, comprising:
a) obtaining a sample containing cells from one or more subjects;
b) depleting αβ T cells from the sample under a condition that generates a depleted cell population comprising NK cells and γδ T cells; and
c) exposing the depleted cell population to an activation condition comprising contacting the depleted cell population with: (a) an antibody or antigen binding fragment thereof that immunospecifically binds to cell adhesion molecule polypeptide CD2; and (b) an antibody or antigen binding fragment thereof that immunospecifically binds to NKp46;
d) exposing the depleted cell population to an expansion condition comprising contacting the depleted cell population with IL-15, thereby generating the composition comprising about 80-99% NK cells.

2. The method of claim 1, wherein the composition comprises about 1% to about 20% γδ T cells.

3. The method of claim 1, wherein the expansion condition comprises incubation of the depleted cell population in the presence of IL-15 for between about one week to about 6 weeks.

4. A method for manufacturing a composition comprising about 70% or more γδ T cells, comprising:
a) obtaining a sample containing cells from one or more subjects;
b) depleting αβ T cells from the sample under a condition that generates a depleted cell population comprising NK cells and γδ T cells; and
c) exposing the depleted cell population to an activation condition comprising contacting the depleted cell population with: (a) an antibody or antigen binding fragment thereof that immunospecifically binds to cell adhesion molecule polypeptide CD2; and (b) an antibody or antigen binding fragment thereof that immunospecifically binds to NKp46;
d) exposing the depleted cell population to an expansion condition comprising contacting the depleted cell population with IL-2, thereby generating the composition comprising about 70-75% γδ T cells.

5. The method of claim 4, wherein the composition comprises about 30% or less NK cells.

6. The method of claim 4, wherein the expansion condition further comprises contacting the depleted cell population with an antibody or antigen binding fragment thereof that immunospecifically binds to CD3 (anti-CD3 antibody).

7. The method of claim 4, wherein the expansion condition comprises incubation of the depleted cell population in the presence of IL-2 and/or anti-CD3 for between about one week to about 6 weeks.

8. The method of claim 1, wherein the activation condition comprises incubation of the depleted cell population in the presence of (a) an antibody or antigen binding fragment thereof that immunospecifically binds to cell adhesion molecule polypeptide CD2; and (b) an antibody or antigen binding fragment thereof that immunospecifically binds to NKp46 for between about 1 hour to about 1 week.

9. The method of claim 1, wherein about 1% or less of the total cells in the composition are αβ T cells.

10. The method of claim 1, wherein depleting αβ T cells from the sample comprises contacting the sample with an antibody that immunospecifically binds to an αβ T cell receptor.

11. The method of claim 1, wherein the antibody or antigen binding fragment thereof that immunospecifically binds to the CD2 is a monoclonal anti-CD2 antibody and/or wherein the antibody or antigen binding fragment thereof that immunospecifically binds to the NKp46 is a monoclonal anti-NKp46 antibody.

12. The method of claim 11, wherein the anti-CD2 antibody and/or anti-NKp46 antibody are soluble.

13. The method of claim 1, wherein the activation condition and/or expansion condition are free of feeder-cells.

14. The method of claim 1, wherein the activation condition and/or expansion condition do not comprise a bisphosphonate.

15. The method of claim 14, further comprising subjecting the depleted cell population to a genetic modification condition after exposing the depleted cell population to an expansion condition.

16. The method of claim 15, wherein the genetic modification comprises introducing an exogenous polynucleotide encoding a chimeric antigen receptor into the cells.

17. The method of claim 1, wherein the activation condition and expansion conditions are performed simultaneously.

18. The method of claim 1, wherein the activation condition and expansion conditions are performed sequentially.

19. The method of claim 1, wherein the sample is peripheral blood, liver tissue, bone marrow, epithelial tissue or cord blood.

20. A cellular composition comprising:
(a) about 80% to about 99% NK cells and about 1% to about 20% γδ T cells;
(b) about 30% or less NK cells and about 70% or more γδ T cells;
(c) about 25% to about 30% NK cells and about 70% to about 75% γδ T cells; or
(d) about 40% to about 45% NK cells and about 55% to about 60% γδ T cells.
